(19) United States
(12) Patent Application Publication
(10) Pub. No.: US 2023/0132318 A1
(43) Pub. Date: Apr. 27, 2023

(54) METHODS OF IMPLANTING ENGINEERED TISSUE CONSTRUCTS

(71) Applicant: Satellite Biosciences, Inc., Cambridge, MA (US)

(72) Inventors: Laura LANDE-DINER, Waban, MA (US); Howard TAKIFF, La Jolla, CA (US); Amanda CHEN, Cambridge, MA (US); Arnav CHHABRA, Cambridge, MA (US); Thomas Jay LOWERY, JR., Sudbury, MA (US); Morgan JAMIEL, Somerville, MA (US); Alison HAYWARD, North Andover, MA (US); Timothy Alfred NORMAN, JR., Dedham, MA (US); Joseph E. MARTURANO, Bedford, MA (US)

(21) Appl. No.: 17/827,128

(22) Filed: May 27, 2022

Related U.S. Application Data
(60) Provisional application No. 63/271,662, filed on Oct. 25, 2021.

(51) Int. Cl. A61L 27/38; A61L 27/22

(52) U.S. Cl. CPC A61L 27/3886; A61L 27/225; A61L 27/3839; A61L 27/3804; A61L 27/3808; A61L 2430/28

(57) ABSTRACT

The present disclosure provides engineered tissue constructs having a population of cells, such as hepatocytes and stromal cells, and methods of making and using the same (e.g., for treating a disease or disorder, such as acute liver failure, a urea cycle disorder, or hyperbilirubinemia (e.g., in a subject having Crigler-Najjar syndrome) in a human subject in need thereof).

### FIELD OF THE INVENTION

The present disclosure concerns engineered tissue constructs and methods of implantation in a subject. The disclosure also provides methods of making and using the same (e.g., for treating a disease or disorder, such as acute liver failure, a urea cycle disorder, or hyperbilirubinemia (e.g., in a subject having Crigler-Najjar syndrome) in a human subject in need thereof).

### BACKGROUND OF THE INVENTION

Many diseases result from damage, malfunction, or loss of a single organ or tissue type. While certain strategies such as organ transplants can be effective, the demand for replacement organs far exceeds availability, resulting in an average of 18 deaths per day in the United States, alone. Tissue therapeutics, including the development of engineered tissue constructs (e.g., cell-based implants), are among the most promising multidisciplinary approaches to fulfill this demand. However, despite significant advances in the fields of cell biology, microfluidics, and engineering, to date, conventional approaches have failed to re-create functional tissues at a scale necessary to impart therapeutic efficacy. Moreover, finding a site within the body that is suitable for vascularization and engraftment has remained challenging. Therefore, there exists an unmet need for engineered tissues that can vascularize in vivo and methods of implantation at sites that support the function of high-density cell masses.

### SUMMARY OF THE INVENTION

The present disclosure provides, inter alia, methods for implanting engineered tissue constructs.

In one aspect, the disclosure provides a method for implanting an engineered tissue construct including a population of mammalian cells in a biocompatible scaffold. The method includes implanting the engineered tissue construct in a human subject in an extraperitoneal space, in an extrapleural space, on a surface of the liver, in a muscle site, in a pleural space, in an omentum site, in a subcutaneous site, on a surface of the pancreas, on a surface of the spleen, on a surface of the kidney, in a bone marrow site, in a bursa site, in a peritoneal cavity site, and/or in a lesser sac site.

In some embodiments, upon implantation, the population of cells are engrafted and vascularized in the subject.

In some embodiments, the primary cells include primary cells expanded in vitro.

In some embodiments, the engineered cells are engineered to express or secrete a protein (e.g., an antibody, a cytokine, an enzyme, a coagulation factor, or a hormone).

In some embodiments, the protein is an endogenous human protein or an engineered protein.

In some embodiments, the engineered tissue construct is implanted in the extraperitoneal space, the extrapleural space, or the surface of the liver.

In some embodiments, the engineered tissue construct is implanted in the extraperitoneal space.

In some embodiments, the extraperitoneal space includes a pre-peritoneal space, a retroperitoneal space, or a subperitoneal space.

In some embodiments, the engineered tissue construct is implanted on the surface of the liver.

In some embodiments, the muscle site is a surface of a muscle, within a muscle sheath, or beneath a muscle.

In some embodiments, the engineered tissue construct is layered on the dome of the liver and/or covered with omentum.

In some embodiments, the peritoneal cavity site is a mesentery site.

In some embodiments, the omentum site includes an omentum pedicle flap, an omentum free flap, an omental bursa, or the omentum in situ.

In some embodiments, the engineered tissue construct is implanted subcutaneously with an omental flap, subcutaneously with an adjuvant, or subcutaneously with an arteriovenous fistula.

In some embodiments, the muscle is a rectus abdominis, an abdominal oblique, a transversus abdominus, a quadriceps femoris, a gluteus maximus, a semimembranosus, a semitendinosus, a biceps femoris, a deltoid, a biceps, or a latissimus dorsi.

In some embodiments, the population of cells includes endocrine, exocrine, paracrine, heterocrine, autocrine, or juxtacrine cells.

In some embodiments, the population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, enteroendocrine cells (e.g., L cells and/or chromaffin cells), ovarian cells, parathyroid cells, thyroid cells, and/or neuronal cells.

In some embodiments, the pituitary cells comprise thyrotropic pituitary cells, lactotropic pituitary cells, corticotropic pituitary cells, somatotropic pituitary cells, and/or gonadotropic pituitary cells.

In some embodiments, the neuronal cells comprise dopaminergic cells.

In some embodiments, the population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the cells are engineered cells, primary cells, or transdifferentiated cells.

In some embodiments, the engineered tissue construct includes two or more populations of cells (e.g., two, three, four, five, six, seven, eight, nine, ten, or more populations of cells).

In some embodiments, the engineered tissue construct includes between 1×10⁶ cells/mL and 1×10⁸ cells/mL, e.g., from 1×10⁶ cells/mL to 10×10⁶ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL) or from 1×10⁷ cells/mL to 1×10⁸ cells/mL (e.g., 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the population of cells includes a population of hepatocytes and a population of stromal cells.

In some embodiments, the hepatocytes include primary human hepatocytes.

In some embodiments, the stromal cells include fibroblasts. In some embodiments, the fibroblasts are normal human dermal fibroblasts or neonatal foreskin fibroblasts.

In some embodiments, the engineered tissue construct further includes a population of endothelial cells. In some embodiments, the population of endothelial cells is arranged as one or more cords.

In some embodiments, the population of cells includes human cells.

In some embodiments, the biocompatible scaffold includes fibrin.

In some embodiments, the engineered tissue construct further includes a reinforcing agent. In some embodiments, the reinforcing agent includes fibrin, surgical mesh, alginate, collagen, poly(ethylene glycol), polyvinylidene acetate, polyvinylidene fluoride, poly(lactic-co-glycolic) acid, or poly (I-lactic acid).

In some embodiments, the engineered tissue construct has a surface area of 10 cm₂ to 2,000 cm², e.g., from 10 cm² to 100 cm² (e.g., 10 cm², 20 cm², 30 cm², 40 cm², 50 cm², 60 cm², 70 cm², 80 cm², 90 cm², or 100 cm²), 100 cm² to 1,000 cm² (e.g., 200 cm², 300 cm², 400 cm², 500 cm², 600 cm², 700 cm², 800 cm², 900 cm², or 1,000 cm²), or 1,000 cm² to 2,000 cm² (e.g., 1,000 cm², 1,100 cm², 1,200 cm², 1,300 cm², 1,400 cm², 1,500 cm², 1,600 cm², 1,700 cm², 1,800 cm², 1,900 cm², or 2,000 cm²).

In some embodiments, the cells are located on a first face of the engineered tissue construct.

In some embodiments, the first face of the engineered tissue construct contacts a site of implantation.

In some embodiments, the cells are located on a first face and a second face of the engineered tissue construct.

In some embodiments, the engineered tissue construct is triangular, rectangular, or circular.

In some embodiments, the method includes implanting a plurality of engineered tissue constructs.

In some embodiments, the method includes implanting a plurality of engineered tissue constructs in a plurality of implantation sites.

In some embodiments, each of the plurality of engineered tissue constructs is implanted in a different site.

In some embodiments, the method includes implanting the engineered tissue construct in a human subject in an extraperitoneal site, the population of cells includes a population of hepatocytes and a population of stromal cells, and the biocompatible scaffold includes fibrin. In another aspect, the disclosure provides a method for implanting an engineered tissue construct that includes a population of human cells in a biocompatible scaffold. The method includes implanting the engineered tissue construct in a human subject in an extraperitoneal space, in an extrapleural space, or on a liver surface. The population of human cells includes a population of hepatocytes. The population of human cells may further include a population of stromal cells. For example, the population of cells may include a population of hepatocytes and a population of stromal cells.

In some embodiments, upon implantation, the population of cells are engrafted and vascularized in the subject.

In some embodiments, the population of human cells includes primary cells, engineered cells, cell aggregates, induced pluripotent stem cell derived cells, embryonic stem cell derived cells, transdifferentiated cells, or a tissue or portion thereof.

In some embodiments, the engineered cells are engineered to express or secrete a protein.

In some embodiments, the protein is an antibody, a cytokine, an enzyme, a coagulation factor, or a hormone.

In some embodiments, the protein is an endogenous human protein or an engineered protein.

In some embodiments, the engineered tissue construct is implanted in the extraperitoneal space.

In some embodiments, the extraperitoneal space is a pre-peritoneal space, a retroperitoneal space, or a subperitoneal space.

In some embodiments, the engineered tissue construct is implanted on the surface of the liver.

In some embodiments, the engineered tissue construct is layered on the dome of the liver and/or covered with omentum.

In some embodiments, the hepatocytes are primary human hepatocytes.

In some embodiments, the stromal cells include fibroblasts. In some embodiments, the fibroblasts are normal human dermal fibroblasts or neonatal foreskin fibroblasts.

In some embodiments, the engineered tissue construct further includes a population of endothelial cells.

In some embodiments, the population of endothelial cells is arranged as one or more cords.

In some embodiments, the biocompatible scaffold includes fibrin.

In some embodiments, the engineered tissue construct further includes a reinforcing agent. The reinforcing agent may include, for example, fibrin, surgical mesh, alginate, collagen, poly(ethylene glycol), polyvinylidene acetate, polyvinylidene fluoride, poly(lactic-co-glycolic) acid, or poly (I-lactic acid).

In some embodiments, the engineered tissue construct has a surface area of 10 cm² to 2,000 cm², e.g., from 10 cm² to 100 cm² (e.g., 10 cm², 20 cm², 30 cm², 40 cm², 50 cm², 60 cm², 70 cm², 80 cm², 90 cm², or 100 cm²), 100 cm² to 1,000 cm² (e.g., 200 cm², 300 cm², 400 cm², 500 cm², 600 cm², 700 cm², 800 cm², 900 cm², or 1,000 cm²), or 1,000 cm² to 2,000 cm² (e.g., 1,000 cm², 1,100 cm², 1,200 cm², 1,300 cm², 1,400 cm², 1,500 cm², 1,600 cm², 1,700 cm², 1,800 cm², 1,900 cm², or 2,000 cm²).

In some embodiments, the engineered tissue construct includes between 1×10⁶ hepatocytes/mL and 1×10⁸ hepatocytes/mL, e.g., from 1×10⁶ hepatocytes/mL to 10×10⁶ hepatocytes/mL (e.g., 1×10⁶ hepatocytes/mL, 2×10⁶ hepatocytes/mL, 3×10⁶ hepatocytes/mL, 4×10⁶ hepatocytes/mL, 5×10⁶ hepatocytes/mL, 6×10⁶ hepatocytes/mL, 7×10⁶ hepatocytes/mL, 8×10⁶ hepatocytes/mL, 9×10⁶ hepatocytes/mL, or 1×10⁷ hepatocytes/mL) or from 1×10⁷ hepatocytes/mL to 1×10⁸ hepatocytes/mL (e.g., 2×10⁷ hepatocytes/mL, 3×10⁷ hepatocytes/mL, 4×10⁷ hepatocytes/mL, 5×10⁷ hepatocytes/mL, 6×10⁷ hepatocytes/mL, 7×10⁷ hepatocytes/mL, 8×10⁷ hepatocytes/mL, 9×10⁷ hepatocytes/mL, or 1×10⁸ hepatocytes/mL).

In some embodiments, the engineered tissue construct has a volume of between 20 mL and 1.2 L, e.g., from 20 mL to 100 mL (e.g., 20 mL, 30 mL, 40 mL, 50 mL, 60 mL, 70 mL, 80 mL, 90 mL, or 100 mL), from 100 mL to 500 mL (e.g., 150 mL, 200 mL, 250 mL, 300 mL, 350 mL, 400 mL, 450 mL, or 500 mL), or from 500 mL to 1.2 L (e.g., 600 mL, 700 mL, 800 mL, 900 mL, 1 L, 1.1 L, or 1.2 L).

In some embodiments, the cells are located on a first face of the engineered tissue construct.

In some embodiments, the first face of the engineered tissue construct contacts a site of implantation.

In some embodiments, the cells are located on a first face and a second face of the engineered tissue construct.

In some embodiments, the method includes implanting a plurality of engineered tissue constructs.

In some embodiments, the method includes implanting a plurality of engineered tissue constructs in a plurality of implantation sites.

In some embodiments, each of the plurality of engineered tissue constructs is implanted in a different site.

In some embodiments, the method treats a liver disease.

The liver disease may be, for example, acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4 (Andersen), hepatic encephalopathy, Hunter syndrome (MPS II), infantile refsum disease, lysosomal acid lipase deficiency (LAL-D) (cholesteryl ester storage disease), maple syrup urine disease, Maroteaux-Lamy (MPS VI), methylmalonic acidemia, ornithine transcarbamylase (OTC) deficiency, propionic acidemia, a urea cycle disorder, alpha-1 antitrypsin deficiency, Niemann-Pick type A/B, Niemann-Pick type C, primary hyperoxaluria type 1, primary hyperoxaluria type 2, primary hyperoxaluria type 3, pyruvate kinase deficiency, liver type, Wilsons disease, D-bifunctional protein deficiency, Gauchers disease, Hurler syndrome (MPS I), hypophosphatasia, morquio A (MPS 4A), morquio B (MPS 4B), multiple acyl-coa dehydrogenase deficiency, sanfilippo (MPS III), acute hepatic porphyrias (AHP), glycogen storage disorder type 3 (Cori), hereditary angioedema (HAE), hereditary hemochromatosis, homocystinuria cystathionine B-synthase deficiency, isovaleric acidemia, N-acetyglutamate synthetase deficiency (NAGS), pseudoxanthoma elasticum, tyrosinemia, 3-methylcrotonyl-CoA carboxylase deficiency (3-MCC), acute fatty liver of pregnancy, congenital factor V deficiency, congenital factor XI deficiency, congenital fibrinogen disorder, corticosteroid-binding globulin deficiency, cutaneous hepatic porphyrias, Fabry disease, factor VII deficiency, factor X deficiency, familial dysbetalipoproteinemia, familial hypertriglyceridemia, familial lipoprotein lipase deficiency, galactosemia, glutaric acidemia type 1, glycogen storage disorder type 1b, glycogen storage disorder type 6 (Hers), hemophilia type A (factor VIII deficiency), hemophilia type B (factor IX), hereditary fructose intolerance, long-chain L-3 hydroxyacyl-CoA dehydrogenase deficiency (LCHAD) deficiency, very long-chain acyl-CoA dehydrogenase (VLCAD) deficiency, 3-hydroxy-3-methylglutaryl-CoA synthase deficiency, abetalipoproteinemia, acetyl-CoA acetyltransferase-2 deficiency, adenosine kinase deficiency, adult polyglucosan body disease, delta-aminolevulinic acid (ALA) dehydratase (ALAD)-deficiency porphyria, alpha-2-plasmin inhibitor deficiency, aminolaevulinic acid dehydratase deficiency porphyria, atransferrinemia, beta-ketothiolase deficiency, bile acid CoA ligase deficiency and defective amidation, carboxypeptidase N deficiency, cerebral creatine deficiency syndrome 1, cerebral creatine deficiency syndrome 2, cerebral creatine deficiency syndrome 3, Chanarin-Dorfman syndrome, cirrhosis-dystonia-polycythemia-hypermanganesemia syndrome, combined oxidative phosphorylation deficiency 1, congenital disorder of deglycosylation, carnitine palmitoyltransferase (CPT) Deficiency, hepatic, type Ia, deoxyguanosine kinase deficiency, formiminoglutamic aciduria, gamma-g lutamylcysteine synthetase deficiency, hepatic lipase deficiency, hepatic tuberculosis, Indian childhood cirrhosis, infantile liver failure syndrome, Lucey-Driscoll syndrome, mitochondrial DNA depletion syndrome, tangier disease, trifunctional protein deficiency, 3-hydroxyacyl-coenzyme A dehydrogenase deficiency, acyl-CoA oxidase deficiency, 3-hydroxy-3-methylglutaric aciduria, 2-methylbutyryl-coa dehydrogenase deficiency, acatalasemia, acquired fructose intolerance, cerebrotendinous xanthomatosis, conjugated hyperbilirubinemia (rotor syndrome), cystic echinococcosis, drug-induced hepatitis, Dubin-Johnson syndrome, focal fatty liver, Gilbert syndrome, glycine n-methyltransferase deficiency, hepatitis A, hepatitis E, liver abscess, liver fibrosis, nodular regenerative hyperplasia, nonalcoholic fatty liver disease (NAFLD), peliosis hepatis, phenylketonuria, short-chain acyl-CoA dehydrogenase deficiency (SCAD), trimethylaminuria, visceral steatosis, vitamin k-dependent clotting factors, combined deficiency of, type 1 and type 2, acute cholangitis/biliary tract infection, alagille syndrome, alphavirus infection, alveolar hydatid disease, benign postoperative, cholestasis, benign recurrent intrahepatic cholestasis, bile acid malabsorption, primary, bile duct cysts, Budd-Chiari syndrome, Caroli disease, cholestasis, clonorchiasis, congenital disorders of glycosylation, congenital hepatic fibrosis, erythropoietic protoporphyrias, familial amyloidosis, familial hypercholanemia, flavivirus infection, hepatic infarction, hepatic veno-occlusive disease, hepatolithiasis, hepatoportal sclerosis, hereditary hemorrhagic telangiectasia, IgG4-related sclerosing cholangitis, intrahepatic cholestasis, isolated neonatal sclerosing cholangitis, non-cirrhotic portal fibrosis, opisthorchiasis, polycystic liver disease, portal hypertension, portal vein thrombosis, primary biliary cholangitis, primary sclerosing cholangitis, progressive familial intrahepatic cholestasis, Reye syndrome, Reynolds syndrome, spontaneous bacterial peritonitis, Von Willebrand disease type 3, biliary atresia, biliary dyskinesia, biliary reflux, cholecystitis, cholelithiasis, alcoholic hepatitis, alcoholic liver disease, autoimmune hepatitis, cystic fibrosis liver disease, hepatitis D, hepatotoxicity, IgG4-related hepatopathy, liver cirrhosis, nonalcoholic steatohepatitis (NASH), hyperammonemia, transjugular intrahepatic portosystemic shunt (TIPS)-induced hyperammonemia, or small for size syndrome.

In some embodiments, the liver disease is acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4 (Andersen), hepatic encephalopathy, Hunter syndrome (MPS II), infantile refsum disease, lysosomal acid lipase deficiency (LAL-D) (cholesteryl ester storage disease), maple syrup urine disease, Maroteaux-Lamy (MPS VI), methylmalonic acidemia, ornithine transcarbamylase (OTC) deficiency, propionic acidemia, or a urea cycle disorder.

In some embodiments, the liver disease is acute liver failure, a urea cycle disorder, or Crigler-Najjar syndrome.

In some embodiments, the engineered tissue construct is implanted using an open surgical procedure or a minimally invasive surgery.

In some embodiments, the engineered tissue construct is affixed by one or more sutures or one or more staples.

In some embodiments, the engineered tissue construct is affixed by suturing adjoining tissue to restrain migration of the engineered tissue construct.

In some embodiments, the engineered tissue construct is implanted at an extraperitoneal site, and the engineered tissue construct is affixed by suturing the muscle fascia to the peritoneum at one or more positions surrounding the engineered tissue construct.

In some embodiments, the engineered tissue construct is not directly sutured.

### Definitions

As used herein, the terms “implanting,” “implantation,” and the like, refer to directly placing one or more engineered tissue constructs in any effective implantation site, such as a site that is suitable for neovascularization in a subject (e.g., a human subject). Exemplary implantation sites include an extraperitoneal space, an extrapleural space, a surface of the liver (e.g., on the surface of a renal capsule), a muscle site (e.g., a surface of a muscle, within a muscle sheath, or beneath a muscle, including but not limited to the following muscles: a rectus abdominis, an abdominal oblique, a transversus abdominus, a quadriceps femoris, a gluteus maximus, a semimembranosus, a semitendinosus, a biceps femoris, a deltoid, a biceps, or a latissimus dorsi), a pleural space, an omentum site (e.g., an omentum pedicle flap, an omentum free flap, an omental bursa, or the omentum in situ), a subcutaneous site, a surface of the pancreas, a surface of the spleen, a surface of the kidney, a bone marrow site, a bursa site, a peritoneal cavity site (e.g., a mesentery site), and/or a lesser sac site, among others.

As used herein, the term “subject” is a mammal. Mammals include, but are not limited to, domesticated animals (e.g., cows, sheep, cats, dogs, and horses), primates (e.g., humans and non-human primates such as monkeys), rabbits, deer, and rodents (e.g., mice and rats). In certain embodiments, the subject is a human.

As used herein, the terms “comprise,” “comprising,” “includes,” and “comprised of” are synonymous with “include,” “including,” “includes” or “contain,” “containing,” “contains” and are inclusive or open-ended terms that specifies the presence of what follows, e.g., a component, and do not exclude or preclude the presence of additional, non-recited components, features, element, members, steps, known in the art or disclosed therein.

As used in the context of the present disclosure, an “engineered tissue construct” refers to a mixture of cultured cells (e.g., hepatocytes (e.g., primary human hepatocytes)) and, optionally, stromal cells (e.g., fibroblasts e.g., neonatal foreskin fibroblasts), and a biocompatible scaffold (e.g., a biocompatible hydrogel scaffold, e.g., fibrin). The relative volume of the engineered tissue construct may be between 0.1 mL to 5 L.

Cells can be from established cell lines, or they can be primary cells, where “primary cells,” “primary cell lines,” and “primary cultures” are used interchangeably herein to refer to cells and cells cultures that have been derived from and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, etc. are most conveniently harvested by biopsy. An appropriate solution can be used for dispersion or suspension of the harvested cells. Such solution will generally be a balanced salt solution, e.g., normal saline, phosphate-buffered saline (PBS), Hanks balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry and Friend, 1969, J. Cell Biol. 43:506-520) which can be adapted for human liver biopsy or autopsy material (e.g., to garner primary human hepatocytes).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a scaffold (e.g., a hydrogel scaffold) is considered “biocompatible” when is it does not exhibit toxicity when introduced into a subject (e.g., a human). In the context of the present disclosure, it is preferable that the biocompatible scaffold does not exhibit toxicity towards the cells of the engineered tissue construct or when implanted in vivo in a subject (e.g., a human). For example, with respect to hepatocytes, hepatotoxicity can be measured, for example, by determining hepatocytes apoptotic death rate (e.g., wherein an increase in apoptosis is indicative of hepatotoxicity), transaminase levels (e.g., wherein an increase in transaminase levels is indicative of hepatotoxicity), ballooning of the hepatocytes (e.g., wherein an increase in ballooning is indicative of hepatotoxicity), microvesicular steatosis in the hepatocytes (e.g., wherein an increase in steatosis is indicative of hepatotoxicity), biliary cells death rate (e.g., wherein an increase in biliary cells death rate is indicative of hepatotoxicity), γ-glutamyl transpeptidase (GGT) levels (e.g., wherein an increase in GGT levels is indicative of hepatotoxicity). A biocompatible scaffold can include, but is not limited to, fibrin and heparin. The biocompatible scaffold maybe a biocompatible hydrogel scaffold.

As used herein, the term “hydrogel” refers to a network of polymer chains that are hydrophilic in nature, such that the material absorbs a high volume of water or other aqueous solution. Hydrogels can include, for example, at least 70% v/v water, at least 80% v/v water, at least 90% v/v water, at least 95%, 96%, 97%, 98% and even 99% or greater v/v water (or other aqueous solution). Hydrogels can include natural or synthetic polymers, the polymeric network often featuring a high degree of crosslinking. Hydrogels also possess a degree of flexibility very similar to natural tissue, due to their significant water content. Hydrogels are particularly useful in tissue engineering applications as scaffolds for culturing cells. In certain embodiments, the hydrogels are made of biocompatible polymers.

The term “adherence material” is a material incorporated into the cell mixture disclosed herein to which a cell or microorganism has some affinity, such as a binding agent. The material can be incorporated, for example, into a hydrogel. The material and a cell or microorganism interact through any means including, for example, electrostatic or hydrophobic interactions, covalent binding, or ionic attachment. The material may include, but is not limited to, antibodies, proteins, peptides, nucleic acids, peptide aptamers, nucleic acid aptamers, sugars, proteoglycans, or cellular receptors.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more; a decrease or an increase of more than 10%, 15%, 20%, 50%, 75%, 100%, or 200%, as compared to a reference; a decrease or an 15 increase by less than 0.01-fold, 0.02-fold, 0.1-fold, 0.3-fold, 0.5-fold, 0.8-fold, or less; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more). A level of a protein may be expressed in mass/vol (e.g., g/dL, mg/mL, μg/mL, or ng/mL) or percentage relative to total protein in a sample.

As used herein, the terms “effective amount,” “therapeutically effective amount,” and the like, when used in reference to an engineered tissue construct described herein, refer to a quantity of hepatocytes and stromal cells (e.g., fibroblasts) in the engineered tissue construct sufficient to, when implanted into the subject (e.g., a mammal e.g., a human) effect beneficial or desired results, such as clinical results. For example, in the context of treating a patient having a urea cycle disorder, these terms refer to an amount of the hepatocytes and stromal cells (e.g., fibroblasts) sufficient to achieve a treatment response as compared to the response obtained without implantation of the engineered tissue construct of interest. An “effective amount,” “therapeutically effective amount,” and the like, of an engineered tissue construct of the present disclosure, also include an amount that results in a beneficial or desired result in a subject as compared to a control.

As used herein, the terms “treat” and “treatment” refer to therapeutic treatment, in which the object is to prevent or slow down (lessen) an undesired physiological change, such as the progression of a disease or disorder, e.g., a liver disease (e.g., acute liver failure, a urea cycle disorder, or hyperbilirubinemia (e.g., in a subject having Crigler-Najjar syndrome)). For example, for a liver disease, beneficial or desired clinical results may include, but are not limited to, the reduction of ammonia, an improvement in a test of gallbladder ejection fraction, or the alleviation of symptoms of acute liver failure, a urea cycle disorder, or hyperbilirubinemia (e.g., in a subject having Crigler-Najjar syndrome). The concentration of ammonia protein or the gallbladder ejection fraction may be determined using assays known in the art, for example, using a hepatobiliary iminodiacetic acid scan.

The term “acute liver failure” includes, but is not limited to, the conditions referred to by the terms hyperacute liver failure, acute liver failure, subacute liver failure, and fulminant hepatic failure (FHF). As used herein, fulminant hepatic failure” or “FHF” are used interchangeable and are defined as the severe impairment of hepatic functions in the absence of pre-existing liver disease. For example, FHF may result from exposure of a susceptible individual to an agent capable of producing serious hepatic injury. Examples of such agents include infectious agents, excessive alcohol, hepatotoxic metabolites, and hepatotoxic compounds (e.g., drugs). Other causes of FHF include congenital abnormalities, autoimmune disease, and metabolic disease. In many cases the precise etiology of FHF is unknown (e.g., idiopathic).

As used herein, the term “urea cycle disorder” refers to any disorder that is caused by a defect or malfunction in the urea cycle. The urea cycle is a cycle of biochemical reactions that produces urea from ammonia, a product of protein catabolism. Specific types of urea cycle disorder include, but are not limited to, phosphate synthetase 1 (CPS1) deficiency, ornithine transcarbamylase (OTC) deficiency, argininosuccinate synthetase (ASS1) deficiency, argininosuccinate lyase (ASL) deficiency, arginase-1 (ARG1) deficiency, N-acetylglutamate synthetase (NAGS) deficiency, ornithine translocase (ORNT1) deficiency, and citrin deficiency. A urea cycle disorder may be characterized by an aberrant level of ammonia (e.g., an ammonia level of greater than or equal to 80 μmol/L).

As used herein, “Crigler-Najjar syndrome” refers to a condition characterized by high levels of bilirubin in the blood (hyperbilirubinemia). Bilirubin is produced when red blood cells are broken down. This substance is removed from the body only after it undergoes a chemical reaction in the liver, which converts the toxic form of bilirubin (called unconjugated bilirubin) to a nontoxic form called conjugated bilirubin. Subjects with Crigler-Najjar syndrome have a buildup of unconjugated bilirubin in their blood (unconjugated hyperbilirubinemia). Crigler-Najjar syndrome is classified into two subtypes, type I and type II. As used herein, “Crigler-Najjar type I” refers to a subtype of Crigler-Najjar syndrome in which mutations in the B-UGT1 gene cause the resulting expressed enzyme, B-UGT, to be completely inactive. Thus, Crigler-Najjar type I patients exhibit a complete absence of B-UGT activity. As used herein, “Crigler-Najjar type II” refers to a subtype of Crigler-Najjar syndrome in which mutations in the B-UGT1 gene cause B-UGT to be partially inactive. Thus, B-UGT activity is reduced in patients with Crigler-Najjar type II and such patients exhibit a strongly reduced bilirubin conjugation capacity compared to healthy subjects.

As used herein, the term “suitable for neovascularization” refers to conditions and/or environmental characteristics fit for the formation of new blood vessels. Generally, neovascularization means the formation of new blood vessels in injured tissue or in tissue not normally containing blood vessels or the formation of novel blood vessels (e.g., arterioles, venules, and capillaries) of a higher density than usual in said tissue. For example, a site that is suitable for neovascularization may have an existing microvessel density of greater than 3.6 vessels/mm² (e.g., greater than 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

As used herein, the terms “liver function test” and “LFT” refer to a hepatic panel (e.g., a group of blood tests that provide information regarding the state of a patients liver). A hepatic panel may include measurement of the level of gamma-glutamyl transferase, the level of alkaline phosphatase, the level of aspartate aminotransferase, the level of alanine aminotransferase, the level of albumin, the level of bilirubin (e.g., total bilirubin, direct bilirubin (also referred to as conjugated bilirubin) and/or indirect bilirubin (also referred to as unconjugated bilirubin)), the prothrombin time, the activated partial thromboplastin time, or a combination thereof.

As used herein, the term “age-adjusted norms” refers to the process of a normalization of data by age, which is a technique that is used to allow populations of subjects to be compared when the age profiles of the populations are different. As used herein, the term “norm” refers to data that does not undergo a normalization by age, as populations of subjects across age profiles are similar.

As used herein, an “extraperitoneal space” refers to a space outside the peritoneal cavity, which is the cavity containing the organs in the abdomen. An extraperitoneal space may be, for example, a preperitoneal space, a retroperitoneal space, or a subperitoneal space. A preperitoneal space is the space between the peritoneum internally and the transversalis fascia externally. Organs in the preperitoneal space include, for example, the liver, spleen, stomach, superior part of the duodenum, jejunum, ileum, transverse colon, sigmoid colon and superior part of the rectum. A retroperitoneal space is the area in the back of the abdomen behind the peritoneum. Organs in the retroperitoneal space include, for example, kidneys, adrenal glands, pancreas, nerve roots, lymph nodes, abdominal aorta, inferior vena cava, and parts of the duodenum and colon. A subperitoneal space is a continuous interconnecting space beneath the peritoneum containing the extraperitoneal space, the ligaments and mesenteries, and their suspended organs. Organs in the subperitoneal space include, for example, the bladder, the cervix of the uterus, and the last part of the rectum. An engineered tissue construct that is implanted in an extraperitoneal space (e.g., preperitoneal space, a retroperitoneal space, or a subperitoneal space) is implanted on a surface within the extraperitoneal space.

As used herein, an “extrapleural space” refers to a region between the inner surface of the ribs and intercostal muscles on one side and the parietal pleura on the other. An engineered tissue construct that is implanted in an extrapleural space is implanted on a surface within the extrapleural space.

As used herein, a “pleural space” or “pleural cavity” is a cavity that exists between the parietal and visceral pleura. An engineered tissue construct that is implanted in a pleural space is implanted on a surface within the pleural space.

As used herein, an “omentum” is a large flat adipose tissue layer nestling on the surface of the intra-peritoneal organs. The greater omentum is a large apron-like fold of visceral peritoneum that hangs down from the stomach. The lesser omentum is the double layer of peritoneum that extends from the liver too the lesser curvature of the stomach, and to the first part of the duodenum.

As used herein, a “lesser sac” or “omental bursa” refers to the cavity in the abdomen that is formed by the lesser and greater omentum.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are a set of schematics showing hepatocytes and stromal cells aggregated in spheroids in a biocompatible scaffold and distributed non-homogenously along the z-axis of the biocompatible scaffold, in a layer at the bottom. FIG. 1A is a set of photomicrographs of increasing dose densities, from left-to-right, of hepatocytes and stromal cells aggregated in spheroids in a biocompatible scaffold and distributed non-homogenously along the z-axis of the biocompatible scaffold, in a layer at the bottom. FIG. 1B is a schematic showing that the same number of aggregates (e.g., including hepatocytes) can be distributed in one layer (top left; e.g., a one-sided engineered tissue construct) or more than one layer (bottom left) in a biocompatible scaffold and that the same dose density of aggregates can be distributed across one layer (top left) or more than one layer (top right) in a biocompatible scaffold (e.g., a two-sided engineered tissue construct).

FIG. 2 is a schematic showing a two-sided engineered tissue construct. Hepatocytes and stromal cells are aggregated in spheroids in a set of two individual biocompatible scaffolds, each of which has the aggregates distributed non-homogenously along the z-axis of the biocompatible scaffold, in a layer at the bottom (step 1). In step 2, the two one-sided engineered tissue constructs are assembled into a two-sided engineered tissue construct with fibrin glue, such that each of the layers of aggregates is outward facing. Two-sided engineered tissue constructs may be produced in other ways. For example, they may be assembled surgically in situ.

FIG. 3 is a set of photomicrographs of a two-sided engineered tissue construct, as described in FIG. 2, with human or bovine fibrin as the biocompatible scaffold, respectively. In both two-sided engineered tissue constructs, both layers of aggregates are facing outward, as described in FIG. 2.

FIG. 4 is a graph showing the serum human albumin levels in NOD-scid IL2Rgamma^{null} (NSG™) mice implanted with an engineered tissue construct including hepatocytes and stromal cells with (“Hepatic Aggregates with Endothelial Cords”) or without endothelial cell cords (“Hepatic Aggregates”).

FIG. 5 is an ultrasound-based image of the vascular volume of the implantation site in NSG™ mice implanted with two anatomically separated engineered tissue constructs. One engineered tissue construct (left circle) contained hepatocytes and stromal cells with endothelial cell cords, while the other (right circle) did not contain endothelial cell cords.

FIG. 6 is a schematic showing the experimental outline of a study to evaluate the prophylactic effect of various dosages of an engineered tissue construct in an in vivo model of acute liver failure. Beginning on day −1 (d−1), each thymidine (TK-NOG) mouse underwent a blood draw. On day 1 (d1) mice in group 3 were implanted with two engineered tissue constructs, including 0.7×10⁶ primary human hepatocytes (PHH)/mouse (low dose “group 3”) or 7×10⁶ PHH/mouse (high dose “group 3). Following implantation, all mice underwent a blood draw on days 6, 11, 16, 21, 30, 35, and 42; as well as a dosing of GCV on days 27 and 32. Mice were sacrificed on day 42.

FIG. 7 is a set of graphs showing the serum levels of liver enzymes alkaline phosphatase (ALP), alanine aminotransferase (ALT), and aspartate aminotransferase (AST), in TK-NOG mice implanted with two low dose (0.7×10⁶ primary human hepatocytes (PHH)/mouse) or high dose (7×10⁶ PHH/mouse) engineered tissue constructs and dosed with GCV, as described in FIG. 6.

FIG. 8 is a graph showing the probability of survival on days post-implantation in TK-NOG mice implanted with low-dose engineered tissue constructs and dosed with GCV, as described in FIG. 6.

FIG. 9 is a graph showing the probability of survival on days post-implantation in TK-NOG mice implanted with high-dose engineered tissue constructs and dosed with GCV, as described in FIG. 6.

FIG. 10 is a graph showing the level of normalized serum ammonia in transgenic B6EiC3Sn a/A-Otc^{spf-ash}/(spf^{ash}) mice that underwent immunosuppression and following implantation with an engineered tissue construct were challenged with NH₄Cl, which was administered intraperitoneally at week 1 and week 4.

FIG. 11 is a graph showing the level of unconjugated bilirubin in the serum of in Gunn rats compared to control Wistar rats.

FIG. 12 is a schematic showing the experimental outline of a study in Gunn rats implanted with engineered tissue constructs described herein, including 11.3×10⁶ primary human hepatocytes/rat (8 grafts per animal with 1.41×10⁶ primary human hepatocytes/graft). Beginning on day −20 (d−20), homozygous Gunn rats and control Wistar rats were subjected to a pre-implantation blood draw every four or five days, followed by immunosuppression on day −7 (d−6), and implantation of eight engineered tissue constructs per rat on day 1 (d1). Blood was collected every four days (e.g., day 5 (d5), day 9 (d9), day 13 (d13), and day 45 (d45)), followed by a terminal bile/blood collection and sacrifice (Sac) on day 49 (d49).

FIG. 13 is a graph showing the level of unconjugated bilirubin and albumin, respectively, in the serum in homozygous male and female Gunn rats, respectively, on the days post-implantation of an engineered tissue construct, as described in FIG. 12. Unconjugated bilirubin levels were normalized to the respective levels in control Wistar rats.

FIG. 14 is a graph showing the level of unconjugated bilirubin over time after implantation of an engineered tissue construct, as described in FIG. 12, in the serum of Gunn rats as a percentage of the control pre-transplant level.

FIG. 15 is a graph showing the concentration of bilirubin diglucuronide (conjugated bilirubin) products in the bile of heterozygous Gunn rats implanted with an engineered tissue construct, as described in FIG. 12, as compared to control animals without an engineered tissue construct.

FIG. 16 is a set of graphs showing the serum human albumin levels in immunocompetent swine (Yorkshire) implanted in the mesentery, omentum, preperitoneal, or subcutaneously, respectively, with an engineered tissue construct including hepatocytes and stromal cells.

FIG. 17 is a photomicrograph showing the expression of hepatocytes and host blood vessels in the graft region of immunocompetent swine (Yorkshire) implanted with an engineered tissue construct including hepatocytes and stromal cells.

FIG. 18 is a schematic drawing showing an engineered tissue construct sized for insertion into a rodent or a human subject. The engineered tissue construct is configured to contain cells on a first face of the construct.

FIG. 19 is a schematic drawing showing an engineered tissue construct sized for insertion into a human subject. The engineered tissue construct is configured to contain cells on a first face of the construct or on a first face and a second face of the construct.

FIG. 20 is a schematic diagram showing illustrating an engineered tissue construct containing cells on a first face and a second face of the construct configured to be implanted an extraperitoneal space between the adipose tissue and the peritoneum of a subject.

FIGS. 21A-21F are a set of images showing an amount of available space for implantation in the extraperitoneal space (FIGS. 21A and 21B), the surface of the liver (FIGS. 210 and 21D), and an extrapleural space (FIGS. 21E and 21F). Unilateral placement positions for the extraperitoneal space and the extrapleural space are shown; however, bilateral placement of engineered tissue constructs may also be performed.

FIG. 22 is a set of images showing the results of a seed orientation study in immunosuppressed Yorkshire swine of a construct containing hepatocytes implanted in a muscle or a peritoneum. On the top are images showing seeds facing the peritoneum. On the bottom are images showing seeds facing the muscle. The left column shows hematoxylin and eosin (H&E) stain; the middle column shows periodic acid-Schiff (PAS) staining; and the right column shows Massons trichome staining.

FIG. 23 is a photomicrograph of a sagittal cross-section taken from a double-layer engineered tissue construct composed of two biocompatible fibrin hydrogel layers, each of which are seeded with hepatocytes and stromal cell spheroids distributed along the entire outer surface of the tissue construct. The panel at the bottom shows a magnified view of the area in the dashed lines.

### DETAILED DESCRIPTION

Engineered tissue constructs described herein include a population of cells (e.g., mammalian cells, e.g., human cells) and a biocompatible scaffold and methods of implantation and treatment using the same. Upon implantation, the cell may be engrafted and vascularized in the subject.

The present disclosure is based, in part, on the observation that implantation of engineered tissue constructs at certain sites as described herein, such as an extraperitoneal space, an extrapleural space, or a liver surface, provide enhanced integration, survival, and vascularization as compared to constructs implanted in other sites. The engineered tissue constructs described herein can be used for methods of treating a disease or disorder, such as liver dysfunction (e.g., a liver disease, e.g., acute liver failure, a urea cycle disorder, or hyperbilirubinemia (e.g., in a subject having Crigler-Najjar syndrome)). The engineered tissue constructs may be used to treat an endocrine disorder, a hormonal deficiency, a protein deficiency, impaired biotransformation, or a disease of impaired protein synthesis.

The sections that follow provide a description of the methods of implantation, engineered tissue constructs, and methods of making and using the same.

### Implantation of Engineered Tissue Constructs

Described herein is a method for implanting an engineered tissue construct that includes a population of human cells in a biocompatible scaffold. The method includes implanting the engineered tissue construct in a human subject in an extraperitoneal space, in an extrapleural space, or on a liver surface. In some embodiments, upon implantation, the population of cells are engrafted and vascularized in the subject.

Also described herein is a method for implanting an engineered tissue construct including a population of mammalian cells in a biocompatible scaffold. The method includes implanting the engineered tissue construct in a human subject in an extraperitoneal space, in an extrapleural space, on a surface of the liver, in a muscle site (e.g., on a surface of a muscle, within a muscle sheath, or beneath a muscle), in a pleural space, in an omentum site, in a subcutaneous site, on a surface of the pancreas, on a surface of the spleen, on a surface of the kidney, in a bone marrow site, in a bursa site, in a peritoneal site (e.g., mesentery), or in a lesser sac site.

The methods of implantation described herein may be used to treat a disease or disorder, such as liver dysfunction, e.g., a liver disease, e.g., acute liver failure, a urea cycle disorder, or hyperbilirubinemia (e.g., in a subject having Crigler-Najjar syndrome). The methods described herein may be used to treat an endocrine disorder, a hormonal deficiency, a protein deficiency, impaired biotransformation, or a disease of impaired protein synthesis.

The engineered tissue constructs described herein can be implanted in a subject. Non-limiting examples of nonhuman subjects include non-human primates, dogs, cats, mice, rats, guinea pigs, rabbits, fowl, pigs, horses, cows, goats, or sheep. In certain embodiments, the subject can be any animal. In certain embodiments, the subject can be any mammal. In certain embodiments, the subject can be a human.

In some embodiments, the subject has an age of between 1 day and 120 years (e.g., between 1 day and 7 days, between 1 day and 1 month, between 1 day and 6 months, between 1 day and 1 year, between 1 day and 2 years, between 1 month and 12 months, between 1 month and 6 months, between 6 months and 2 years, between 1 year and 2 years, between 1 year and 5 years, between 1 year and 10 years, between 10 years and 20 years, between 10 years and 50 years, between 50 years and 80 years, or between 60 years and 90 years). In some embodiments, the subject has an age of between 1 day and 1 year. In some embodiments, the subject is a newborn or an infant.

In some embodiments, the engineered tissue construct is implanted into a subject in an extraperitoneal space, in an extrapleural space, on a surface of the liver, in a muscle site (e.g., a surface of a muscle, within a muscle sheath, or beneath a muscle), in a pleural space, in an omentum site, in a subcutaneous site, on a surface of the pancreas, on a surface of the spleen, on a surface of the kidney, in a bone marrow site, in a bursa site, in a peritoneal site, or in a lesser sac site

In some embodiments, the engineered tissue construct is implanted into the subject in an extraperitoneal space, in an extrapleural space, or on a liver surface. In some embodiments, the engineered tissue construct is implanted in the extraperitoneal space. In some embodiments, the engineered tissue construct is implanted in a pre-peritoneal space, a retroperitoneal space, or a subperitoneal space.

In some embodiments, the engineered tissue construct is implanted on the surface of the liver.

In some embodiments, the engineered tissue construct is layered on the dome of the liver and/or covered with omentum.

In some embodiments, the muscle site is a surface of a muscle. In some embodiments, the muscle site is within a muscle sheath. In some embodiments, the muscle site is beneath a muscle.

In some embodiments, the omentum site includes an omentum pedicle flap, an omentum free flap, an omental bursa, or the omentum in situ.

In some embodiments, the engineered tissue construct is implanted subcutaneously with an omental flap, subcutaneously with an adjuvant, or subcutaneously with an arteriovenous fistula.

In some embodiments, the engineered tissue construct is implanted into the subject in an implantation site selected from the group consisting of the peritoneum, an extraperitoneal site (e.g., retroperitoneum), pre-perintoneal space, or a subperitoneal space), peritoneal cavity (e.g., omentum or mesentery), rectus abdominis muscle, abdominal oblique muscle, quadriceps femoris muscle, gluteus maximus, a hamstring muscle (e.g., a semimembranosus, a semitendinosus, or a biceps femoris), deltoid, biceps, latissimus dorsi, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the mesentery is the small intestinal mesentery. In some embodiments, the engineered tissue construct is implanted into the subject as a pedicled omental wrap or an omental wrap.

In some embodiments, one or more engineered tissue constructs is implanted in one or more implantation sites selected from the group consisting of the peritoneum, retroperitoneum, peritoneal cavity (e.g., omentum or mesentery), rectus abdominis muscle, abdominal oblique muscle (including an internal oblique muscle or an external oblique muscle), transversus abdominis muscle, quadriceps femoris muscle, gluteus maximus, a hamstring muscle (e.g., a semimembranosus muscle, a semitendinosus muscle, or a biceps femoris muscle), deltoid, biceps, latissimus dorsi, extraperitoneal fat, and renal capsule; an extraperitoneal site, a surface of the liver, or an extrapleural site; or a site that is suitable for neovascularization. For example, in some embodiments, the engineered tissue construct is implanted in the retroperitoneum. For example, in some embodiments, two engineered tissue constructs are implanted bilaterally on an omentum site.

The engineered tissue construct can be implanted in any suitable manner, often with pharmaceutically acceptable carriers. In some embodiments, the engineered tissue construct is implanted on a surface of a tissue or organ. In some embodiments, the engineered tissue construct is implanted on an orthotopic site. In other embodiments, the engineered tissue construct is implanted on an ectopic site.

Any suitable approach may be used to perform the implantation. For example, the engineered tissue construct may be implanted using an open surgical procedure or a minimally invasive surgery. In some embodiments, the engineered tissue construct may be implanted using an open surgical procedure.

In other embodiments, the engineered tissue construct may be implanted using a minimally invasive surgery.

The engineered tissue construct may be affixed to the subject using any suitable approach. For example, the engineered tissue construct may be affixed using sutures, staples, or by welding (e.g., laser tissue welding). In some embodiments, the engineered tissue construct is affixed by one or more sutures or one or more staples. In some embodiments, the engineered tissue construct is affixed by suturing adjoining tissue to restrain migration of the engineered tissue construct. For example, in some embodiments, the engineered tissue construct is implanted at an extraperitoneal site, and the engineered tissue construct is affixed by suturing the muscle fascia to the peritoneum at one or more positions surrounding the engineered tissue construct. In some embodiments, the engineered tissue construct is not directly sutured or stapled (in other words, the sutures or staples do not penetrate the engineered tissue construct itself). In some embodiments, the engineered tissue construct is implanted in a site that is suitable for neovascularization. In some embodiments, a site that it suitable for neovascularization is one having a microvessel density of from 3.6 vessels/mm² to 4500 vessels/mm² (e.g., 3.7 vessels/mm² to 4000 vessels/mm², 3.8 vessels/mm² to 3500 vessels/mm², 3.9 vessels/mm² to 3000 vessels/mm², 4 vessels/mm² to 2500 vessels/mm², 5 vessels/mm² to 2000 vessels/mm², 10 vessels/mm² to 1000 vessels/mm², or 100 vessels/mm²).

In some embodiments, a site that is suitable for neovascularization may have an existing microvessel density of greater than 3.7 vessels/mm². In some embodiments, a site that is suitable for neovascularization may have an existing microvessel density of greater than 4 vessels/mm². In some embodiments, a site that is suitable for neovascularization may have an existing microvessel density of greater than 4.5 vessels/mm². In some embodiments, a site that is suitable for neovascularization may have an existing microvessel density of greater than 5 vessels/mm². In some embodiments, a site that is suitable for neovascularization may have an existing microvessel density of greater than 10 vessels/mm². In some embodiments, a site that is suitable for neovascularization may have an existing microvessel density of greater than 50 vessels/mm². In some embodiments, a site that is suitable for neovascularization may have an existing microvessel density of greater than 100 vessels/mm². In some embodiments, a site that is suitable for neovascularization may have an existing microvessel density of greater than 1000 vessels/mm². In some embodiments, a site that is suitable for neovascularization may have an existing microvessel density of greater than 4500 vessels/mm². Autologous, allogenic or xenogenic cells may be used. The cells may be implanted in any physiologically acceptable medium. In one embodiment, the cells are cryopreserved in 5-20% DMSO, 5% dextrose and autologous serum. As is familiar to those of skill in the art, dosage of the cells of the present invention to be implanted in vivo is determined with reference to various parameters, including the species of the host, the age, weight, and disease status. Dosage also depends upon the location to be targeted within the subject. For example, implantation of the engineered tissue construct into the omentum may require different dosages than implantation to the mesentery. The dosage is preferably chosen so that implantation causes an effective result, which can be measured by molecular assays (e.g., a liver function test) or by monitoring a suitable symptom in the subject (e.g., symptoms of Crigler-Najjar).

In some embodiments, the method further comprises administering an immunosuppressive or immunomodulatory drug to modulate an immune response. In some embodiments, the immune response is a humoral response or antibody-mediated response.

In some embodiments, the implantation method prevents graft rejection or promotes graft survival.

The engineered tissue constructs disclosed herein can be administered in combination with one or more additional immunosuppressive therapies including; but not limited to drugs which inhibit T-cell activation (e.g.; calcineurin inhibitors (CNI)); systemic immunosuppressants for universal transplant immunotolerance (corticosteroids such as methylprednisolone (MEDROL® or; SOLU-MEDROL®); prednisone or prednisolone); CNI such as tacrolimus (PROGRAF® or; ASTAFRAF®); cyclosporine (NEORAL®; SANDIMMUNE® or; GENGRAF®); co-stimulation blockade therapy such as Abatacept (ORENCIA®) and Belatacept (NULOJIX®); anti-metabolites such as Mycophenolate motefil (CELLCEPT® or; MYFORTIC®); Azathioprine (IMURAN®); mTORI such as Sirolimus (RAPAMUNE®); Everolimus (AFINITOR®); T-cell depleting monoclonal antibodies such as muromonab-CD3 (OKT3); Alemtuzumab (Campath® or; LEMTRADA®); ATG (THYMOBLOBULIN® or; ATGAM®); B-cell depleting monoclonal antibodies such as rituximab (RITUXAN®); proteasome inhibitors such as Bortezomib (VELCADE®); IL-2-Ra monoclonal antibodies such as daclizumab (ZENAPAX®); Basiliximab (SIMULECTe); lymphocyte integrin blockade monoclonal antibodies such as Natalizumab (TYSABRI®); N-Acetyl Cysteine (NAC); hepatitis B vaccine (HEPLISAV-B®); glecaprevir and pibrentasvir (MAVYRET®); sofosbuvir (VOSEVI®); obeticholic acid (OCALIVA®); elbasvir and grazoprevir (ZEPATIER®); cholic acid (CHOLBAM®); daclatasvir (DAKLINZA®); ombitasvir, paritaprevir, and ritonavir (TECHNIVIE™); simeprevir (OLYSIO™); sofosbuvir (SOVALDI®); telaprevir (INCIVEK™); boceprevir (VICTRELIS™); tenofovir disoproxil fumarate (VIREAD®); telbivudine (TYZEKA™); entecavir (BARACLUDE™); adefovir (HEPSERA®); peginterferon alfa-2a (PEGASYS®); peginterferon alfa-2b (PEGINTRON®); or ribavirin and twinrix. Additional agents include gliltazones and vitamin E.

In some embodiments, the engineered tissue constructs disclosed herein can be administered in combination with one or more additional immunosuppressive therapies including but not limited to a PEGylated anti-CD28 monovalent monoclonal antibody fragment (e.g., anti-human CD28 FR104) or domain antibody such as lulizumab (BMS-931699), an IL-2Ra specific antibody for Treg expansion (e.g., a Fc IL-2 mutein (e.g., AMG-592)), a PEGylated IL-2 antibody, a humanized IgG1 anti-CD40L antagonist (e.g., AT-1501), a bivalent anti-CD40L domain antibody such as letolizumab (BMS-986004), an Fc silent human IgG1 anti-CD40 antibody such as VIB4920 or iscalimab (CFZ533), imlifidase, or a human anti-IL6 monoclonal antibody such as clazakizumab (CSL300).

In some embodiments, following implantation of the engineered tissue construct, the engineered tissue construct persists for at least two weeks (e.g., at least three weeks, one month, two months, three months, four months, five months, six months, seven months, ten months, eleven months, one year, five years, ten years, or the lifetime of a patient in which the engineered tissue construct is implanted into). For example, in some embodiments, following implantation of the engineered tissue construct, the engineered tissue construct persists for at least three weeks. In some embodiments, following implantation of the engineered tissue construct, the engineered tissue construct persists for at least one month. In some embodiments, following implantation of the engineered tissue construct, the engineered tissue construct persists for at least two months. In some embodiments, following implantation of the engineered tissue construct, the engineered tissue construct persists for at least three months. In some embodiments, following implantation of the engineered tissue construct, the engineered tissue construct persists for at least four months. In some embodiments, following implantation of the engineered tissue construct, the engineered tissue construct persists for at least five months. In some embodiments, following implantation of the engineered tissue construct, the engineered tissue construct persists for at least six months. In some embodiments, following implantation of the engineered tissue construct, the engineered tissue construct persists for at least seven months. In some embodiments, following implantation of the engineered tissue construct, the engineered tissue construct persists for at least eight months. In some embodiments, following implantation of the engineered tissue construct, the engineered tissue construct persists for at least nine months. In some embodiments, following implantation of the engineered tissue construct, the engineered tissue construct persists for at least ten months. In some embodiments, following implantation of the engineered tissue construct, the engineered tissue construct persists for at least eleven months. In some embodiments, following implantation of the engineered tissue construct, the engineered tissue construct persists for at least one year. In some embodiments, following implantation of the engineered tissue construct, the engineered tissue construct persists for at least five years. In some embodiments, following implantation of the engineered tissue construct, the engineered tissue construct persists for at least ten years. In some embodiments, following implantation of the engineered tissue construct, the engineered tissue construct persists for the lifetime of a patient in which the engineered tissue construct is implanted into. In some embodiments, following implantation of the engineered tissue construct, the engineered tissue construct persists until an organ transplant occurs (e.g., until the patient receives a liver transplant).

### Engineered Tissue Construct

Engineered tissue constructs described herein include a population of cells (e.g., mammalian cells, e.g., human cells) and a biocompatible scaffold.

In some embodiments, the engineered tissue construct includes two or more populations of cells (e.g., two, three, four, five, six, seven, eight, nine, ten, or more populations of cells).

The shape of the engineered tissue construct may depend on the site of implantation and/or the disease or disorder to be treated. In some embodiments, the engineered tissue construct is triangular, rectangular (e.g., square), or circular. In some embodiments, the engineered tissue construct has a thickness than is substantially less than its length and width. For example, the engineered tissue construct may be substantially flat, e.g., a flat rectangle, triangle, or disc shape.

The engineered tissue construct may have a length, width, and thickness that are each, independently, from 0.1 mm to 100 cm, e.g., from 0.1 mm to 1 mm (e.g., 0.1 mm, 0.2 mm, 0.3 mm, 0.4 mm, 0.5 mm, 0.6 mm, 0.7 mm, 0.8 mm, 0.9 mm, or 1 mm), from 1 mm to 1 cm (e.g., 1 mm, 2 mm, 3 mm, 4 mm, 5 mm, 6 mm, 7 mm, 8 mm, 9 mm, or 1 cm), from 1 cm to 10 cm (e.g., 1 cm, 2 cm, 3 cm, 4 cm, 5 cm, 6 cm, 7 cm, 8 cm, 9 cm, or 10 cm), or from 10 cm to 100 cm (e.g., 20 cm, 30 cm, 40 cm, 50 cm, 60 cm, 70 cm, 80 cm, 90 cm, or 100 cm). In some embodiments, the thickness of the engineered tissue construct is from 0.1 mm to 1 cm, e.g., from 0.1 mm to 1 mm (e.g., 0.1 mm, 0.2 mm, 0.3 mm, 0.4 mm, 0.5 mm, 0.6 mm, 0.7 mm, 0.8 mm, 0.9 mm, or 1 mm) or from 1 mm to 1 cm (e.g., 1 mm, 2 mm, 3 mm, 4 mm, 5 mm, 6 mm, 7 mm, 8 mm, 9 mm, or 1 cm), and the length and/or width are each, independently, from 1 cm to 10 cm (e.g., 1 cm, 2 cm, 3 cm, 4 cm, 5 cm, 6 cm, 7 cm, 8 cm, 9 cm, or 10 cm) or from 10 cm to 100 cm (e.g., 20 cm, 30 cm, 40 cm, 50 cm, 60 cm, 70 cm, 80 cm, 90 cm, or 100 cm). In some embodiments, the length and width are each, independently, at least 5-fold (e.g., at least 5-fold, at least 6-fold, 7-fold, 8-fold, 9-fold, 10-folder, 20-fold, 30-fold, 40-fold, 50-fold, or more) greater than the thickness of the engineered tissue construct.

In some embodiments, the engineered tissue construct has a surface area of 10 cm² to 2,000 cm², e.g., from 10 cm² to 100 cm² (e.g., 10 cm², 20 cm², 30 cm², 40 cm², 50 cm², 60 cm², 70 cm², 80 cm², 90 cm², or 100 cm²), 100 cm² to 1,000 cm² (e.g., 100 cm², 200 cm², 300 cm², 400 cm², 500 cm², 600 cm², 700 cm², 800 cm², 900 cm², or 1,000 cm²), or 1,000 cm² to 2,000 cm² (e.g., 1,000 cm², 1,100 cm², 1,200 cm², 1,300 cm², 1,400 cm², 1,500 cm², 1,600 cm², 1,700 cm², 1,800 cm², 1,900 cm², or 2,000 cm²).

In some embodiments, the cells are present (e.g., encapsulated) on a first face of the engineered tissue construct. In some embodiment, the cells are present (e.g., encapsulated) on a first face and a second face of the engineered tissue construct. In some embodiments, the first face of the engineered tissue construct containing the encapsulated cells contacts a site of implantation.

In some embodiments, the method includes implanting a plurality of engineered tissue constructs. For example, in some embodiments, the engineered tissue construct has a thickness than is substantially less than its length and width (e.g., a substantially flat rectangle, triangle, or disc shape) and a stack of engineered tissue constructs is implanted in a site of implantation. In other examples, the members of the plurality may be implanted in different implantation sites.

In another aspect, the cells are provided in the form of an aggregate (e.g., a spheroidal aggregate). In some embodiments, the cell populations are admixed under conditions which cause the two cell populations to form aggregates. In some embodiments, the cell populations are admixed using tissue fabrication techniques. In some embodiments, two or more cell populations are co-cultured. In some embodiments, the two or more cell populations are cocultured by hanging drop, microwell molding, non-adhesive surfaces, spheroid suspension culture using a spinner flask, vertical wheel bioreactor, horizontal wheel bioreactor, or a microfluidic spheroid system. Additional methods include those using acoustical waves and using positively charged surfaces on a plate.

In other aspects, the compositions provided herein can contain additional components, including but not limited to, growth factors, ligands, cytokines, drugs, and the like. In some embodiments, the cell mixtures can include molecules which elicit additional microenvironmental cues such as small molecules or growth factors which stimulate or enhance proliferation and expansion of a cell population.

The properties of the cell aggregates of the present disclosure can be varied to suit a particular application. In certain embodiments, the density of the cell aggregates can be changed. In certain embodiments, cell aggregates of different diameters can be fabricated. In certain embodiments, the overall network organization of the one or more cell aggregates can be defined, for example, by the number, three-dimensional organization, alignment, diameters, density, and the like.

In certain embodiments, the engineered tissue construct can contain one or more bioactive substances. Examples of bioactive substance(s) include, but are not limited to, hormones, neurotransmitters, growth factors, hormone, neurotransmitter or growth factor receptors, interferons, interleukins, chemokines, cytokines, colony stimulating factors, chemotactic factors, extracellular matrix components, and adhesion molecules, ligands and peptides; such as growth hormone, parathyroid hormone bone morphogenetic protein, transforming growth factor-alpha, TGF-beta1, TGF-beta2, stromal cell growth factor, granulocyte/macrophage colony stimulating factor, epidermal growth factor, platelet derived growth factor, insulin-like growth factor, scatter factor/hepatocyte growth factor, fibrin, dextran, matrix metalloproteinases, collagen, fibronectin, vitronectin, hyaluronic acid, an RGD-containing peptide or polypeptide, an angiopoietin and vascular endothelial cell growth factor.

In certain embodiments, the engineered tissue constructs disclosed herein include one or more adherence materials to facilitate maintenance of the desired phenotype of the grafted cells in vivo. The material may include, but is not limited to, antibodies, proteins, peptides, nucleic acids, peptide aptamers, nucleic acid aptamers, sugars, proteoglycans, or cellular receptors.

The type of adherence materials (e.g., extra-cellular matrix (ECM) materials, sugars, proteoglycans etc.) will be determined, in part, by the cell type or types (e.g., hepatocytes and stromal cells) to be cultured. ECM molecules found in a cells native microenvironment are useful in maintaining the function of both primary cells, precursor cells, and/or cell lines.

In some embodiments, the engineered tissue construct further includes a biocompatible scaffold (e.g., biocompatible hydrogel scaffold). For example, in some embodiments, the biocompatible scaffold is fibrin. In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel comprising the synthetic polymer described herein can bind various bioactive agents, e.g., growth factors, thereby preventing the bioactive agents from diffusing away and maintaining the bioactive agents at a high concentration locally, so that they can act on cells and promote various cell functions.

In some embodiments, the engineered tissue construct includes between 1×10⁶ cells/mL and 1×10⁸ cells/mL, e.g., from 1×10⁶ cells/mL to 10×10⁶ cells/mL (e.g., 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, or 1×10⁷ cells/mL) or from 1×10⁷ cells/mL to 1×10⁸ cells/mL (e.g., 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, or 1×10⁸ cells/mL).

In some embodiments, the engineered tissue construct is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume. In some embodiments, the engineered tissue construct has a volume of between 20 mL and 1.2 L, e.g., from 20 mL to 100 mL (e.g., 20 mL, 30 mL, 40 mL, 50 mL, 60 mL, 70 mL, 80 mL, 90 mL, or 100 mL), from 100 mL to 500 mL (e.g., 100 mL, 150 mL, 200 mL, 250 mL, 300 mL, 350 mL, 400 mL, 450 mL, or 500 mL), or from 500 mL to 1.2 L (e.g., 500 mL, 600 mL, 700 mL, 800 mL, 900 mL, 1 L, 1.1 L, or 1.2 L). For example, in some embodiments, the engineered tissue construct is from 0.2 mL to 5 L in volume. In some embodiments, the engineered tissue construct is from 0.3 mL to 5 L in volume. In some embodiments, the engineered tissue construct is from 0.4 mL to 5 L in volume. In some embodiments, the engineered tissue construct is from 0.5 mL to 5 L in volume. In some embodiments, the engineered tissue construct is from 1 mL to 5 L in volume. In some embodiments, the engineered tissue construct is from 5 mL to 5 L in volume. In some embodiments, the engineered tissue construct is from 10 mL to 5 L in volume. In some embodiments, the engineered tissue construct is from 100 mL to 5 L in volume. In some embodiments, the engineered tissue construct is from 1 mL to 5 L in volume. In some embodiments, the engineered tissue construct is from 2 mL to 5 L in volume. In some embodiments, the engineered tissue construct is from 3 mL to 5 L in volume. In some embodiments, the engineered tissue construct is from 4 mL to 5 L in volume.

In some embodiments, the engineered tissue construct further includes a reinforcing agent. In some embodiments the reinforcing agent is selected from the list including fibrin, surgical mesh, alginate, collagen, poly(ethylene glycol), polyvinylidene acetate (PVDA), polyvinylidene fluoride (PVDF), poly(lactic-co-glycolic) acid (PLGA), and poly (I-lactic acid) (PLLA). In some embodiments the reinforcing agent is fibrin. In some embodiments the reinforcing agent is surgical mesh. In some embodiments the reinforcing agent is alginate. In some embodiments the reinforcing agent is collagen. In some embodiments the reinforcing agent is poly(ethylene glycol). In some embodiments the reinforcing agent is PVDA. In some embodiments the reinforcing agent is PVDF. In some embodiments the reinforcing agent is PLGA. In some embodiments the reinforcing agent is PLLA. In some embodiments the reinforcing agent is any suitable agent.

In some embodiments, the engineered tissue construct may be any shape (e.g., cylindrical, square, or square with rounded corners).

In some embodiments, the engineered tissue construct has a serpentine topography (e.g., to increase surface area).

### Cell Populations

Cell populations may be optimized to maintain the appropriate morphology, phenotype, and cellular function conducive to use in the methods of the disclosure. Cell populations of the engineered tissue constructs described herein may include a population of mammalian (e.g., human) cells. The population may include primary cells, engineered cells, cell aggregates, induced pluripotent stem cell (iPSC)-derived cells, embryonic stem cell-derived cells, transdifferentiated cells, or a tissue or portion thereof. The cells may be primary cells, e.g., primary cells that are expanded in vitro. In some embodiments, the population of cells includes endocrine, exocrine, paracrine, heterocrine, autocrine, or juxtacrine cells.

In some embodiments, the population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, enteroendocrine cells (e.g., L cells and/or chromaffin cells), ovarian cells, parathyroid cells, thyroid cells, and/or neuronal cells.

In some embodiments, the pituitary cells comprise thyrotropic pituitary cells, lactotropic pituitary cells, corticotropic pituitary cells, somatotropic pituitary cells, and/or gonadotropic pituitary cells.

In some embodiments, the neuronal cells comprise dopaminergic cells.

In some embodiments, the population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the population of cells are primary cells. In some embodiments the first population of cells are induced pluripotent (iPSC)-derived cells or embryonic stem cells (ESC)-derived cells.

In some embodiments, the cells are engineered cells, primary cells, or transdifferentiated cells.

In some embodiments, the engineered tissue construct includes two or more populations of cells (e.g., two, three, four, five, six, seven, eight, nine, ten, or more populations of cells).

In some embodiments, the population of cells includes a population of hepatocytes and a population of stromal cells.

In some embodiments, the engineered cells are engineered to express or secrete a protein, such as an antibody, a cytokine, an enzyme, a coagulation factor, or a hormone. The protein may be, for example, an endogenous human protein or an engineered protein.

In some embodiments, the engineered tissue constructs include hepatocytes and stromal cells, which may be optimized to maintain the appropriate morphology, phenotype, and cellular function conducive to use in the methods of the disclosure. For example, primary human hepatocytes or neonatal foreskin stromal cells can be isolated and/or pre-cultured under conditions optimized to ensure that the respective cells of choice initially have the desired morphology, phenotype, and cellular function and, thus, are poised to maintain said morphology, phenotype and/or function in vivo following implantation of the engineered tissue constructs described herein.

### Hepatocytes

The engineered tissue construct described herein may include hepatocytes. In some embodiments, the hepatocytes are primary human hepatocytes (PHH). In some embodiments, the hepatocytes are derived from stem cells (e.g., induced pluripotent stem cells).

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). For example, in some embodiments, the density of hepatocytes is 0.2 M/mL to 149 M/mL. In some embodiments, the density of hepatocytes is 0.3 M/mL to 148 M/mL. In some embodiments, the density of hepatocytes is 0.4 M/mL to 147 M/mL. In some embodiments, the density of hepatocytes is 0.5 M/mL to 146 M/mL. In some embodiments, the density of hepatocytes is 1 M/mL to 145 M/mL. In some embodiments, the density of hepatocytes is 5 M/mL to 140 M/mL. In some embodiments, the density of hepatocytes is 10 M/mL to 100 M/mL. In some embodiments, the density of hepatocytes is 20 M/mL to 50 M/mL. In some embodiments, the density of hepatocytes is 30 M/mL to 40 M/mL.

In some embodiments, the engineered tissue construct includes a population of hepatocytes in an amount of from 3×10⁵ to 1.8×10¹¹ (e.g., from 4×10⁵ to 1.8×10¹¹, from 5×10⁵ to 1.8×10¹¹, from 6×10⁵ to 1.8×10¹¹, from 7×10⁵ to 1.8×10¹¹, from 8×10⁵ to 1.8×10¹¹, from 9×10⁵ to 1.8×10¹¹, from 1×10⁶ to 1.8×10¹¹, from 2×10⁶ to 1.8×10¹¹, from 3×10⁶ to 1.8×10¹¹, from 4×10⁶ to 1.8×10¹¹, from 5×10⁶ to 1.8×10¹¹, from 6×10⁶ to 1.8×10¹¹, from 7×10⁶ to 1.8×10¹¹, from 8×10⁶ to 1.8×10¹¹, from 9×10⁶ to 1.8×10¹¹, from 1×10⁷ to 1.8×10¹¹, from 2×10⁷ to 1.8×10¹¹, from 1.8×10⁷ to 1.8×10¹¹, from 4×10⁷ to 1.8×10¹¹, from 5×10⁷ to 1.8×10¹¹, from 6×10⁷ to 1.8×10¹¹, from 7×10⁷ to 1.8×10¹¹, from 8×10⁷ to 1.8×10¹¹, from 9×10⁷ to 1.8×10¹¹, from 1×10⁸ to 1.8×10¹¹, from 2×10⁸ to 1.8×10¹¹, from 3×10⁸ to 1.8×10¹¹, from 4×10⁸ to 1.8×10¹¹, from 5×10⁸ to 1.8×10¹¹, from 6×10⁸ to 1.8×10¹¹, from 7×10⁸ to 1.8×10¹¹, from 8×10⁸ to 1.8×10¹¹, from 9×10⁸ to 1.8×10¹¹, from 1×10⁹ to 1.8×10¹¹, from 2×10⁹ to 1.8×10¹¹, from 3×10⁹ to 1.8×10¹¹, from 4×10⁹ to 1.8×10¹¹, from 5×10⁹ to 1.8×10¹¹, from 6×10⁹ to 1.8×10¹¹, from 7×10⁹ to 1.8×10¹¹, from 8×10⁹ to 1.8×10¹¹, from 9×10⁹ to 1.8×10¹¹, from 1×10¹⁰ to 1.8×10¹¹, from 2×10¹⁰ to 1.8×10¹¹, from 3×10¹⁰ to 1.8×10¹¹, from 4×10¹⁰ to 1.8×10¹¹, from 5×10¹⁰ to 1.8×10¹¹, from 6×10¹⁰ to 1.8×10¹¹, from 7×10¹⁰ to 1.8×10¹¹, from 8×10¹⁰ to 1.8×10¹¹, from 9×10¹⁰ to 1.8×10¹¹, or from 1×10¹¹ to 1.8×10¹¹) hepatocytes.

### Stromal Cells

The engineered tissue constructs described herein may include stromal cells, (e.g., fibroblasts). In some examples, the engineered tissue constructs described herein include stromal cells, (e.g., fibroblasts). In some embodiments, the fibroblasts are human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). In some embodiments, the fibroblasts are human dermal fibroblasts. In some embodiments, the fibroblasts are normal human dermal fibroblasts. In some embodiments, the fibroblasts are neonatal foreskin fibroblasts. In some embodiments, the fibroblasts human lung fibroblasts. In some embodiments, the fibroblasts are human ventricular cardiac fibroblasts. In some embodiments, the fibroblasts are human atrial cardiac fibroblasts. In some embodiments, the fibroblasts are human uterine fibroblasts. In some embodiments, the fibroblasts are human bladder fibroblasts. In some embodiments, the fibroblasts are human gingival fibroblasts. In some embodiments, the fibroblasts are human pericardial fibroblasts. In some embodiments, the fibroblasts are human gall bladder fibroblasts. In some embodiments, the fibroblasts are human portal vein fibroblasts. In some embodiments, the fibroblasts are vas deferens fibroblasts.

In some embodiments, the population of stromal cells includes from 6×10³ to 1.8×10¹² e.g., from 1 to 1.8×10¹², from 10 to 1.8×10¹², from 100 to 1.8×10¹², from 1×10³ to 1.8×10¹², from 2×10³ to 1.8×10¹², from 3×10³ to 1.8×10¹², from 4×10³ to 1.8×10¹², from 5×10³ to 1.8×10¹², from 6×10³ to 1.8×10¹², from 7×10³ to 1.8×10¹², from 8×10³ to 1.8×10¹², from 9×10³ to 1.8×10¹², from 1×10⁴ to 1.8×10¹², from 2×10⁴ to 1.8×10¹², from 3×10⁴ to 1.8×10¹², from 4×10⁴ to 1.8×10¹², from 5×10⁴ to 1.8×10¹², from 6×10⁴ to 1.8×10¹², from 7×10⁴ to 1.8×10¹², from 8×10⁴ to 1.8×10¹², from 9×10⁴ to 1.8×10¹², from 1×10⁵ to 1.8×10¹², from 2×10⁵ to 1.8×10¹², from 3×10⁵ to 1.8×10¹², from 4×10⁵ to 1.8×10¹², from 5×10⁵ to 1.8×10¹², from 6×10⁵ to 1.8×10¹², from 7×10⁵ to 1.8×10¹², from 8×10⁵ to 1.8×10¹², from 9×10⁵ to 1.8×10¹², from 1×10⁶ to 1.8×10¹², from 2×10⁶ to 1.8×10¹², 3×10⁶ to 1.8×10¹², 4×10⁶ to 1.8×10¹², 5×10⁶ to 1.8×10¹², 6×10⁶ to 1.8×10¹², 7×10⁶ to 1.8×10¹², 8×10⁶ to 1.8×10¹², 9×10⁶ to 1.8×10¹², from 1×10⁷ to 1.8×10¹², from 2×10⁷ to 1.8×10¹², from 1 8×10⁷ to 1.8×10¹², from 4×10⁷ to 1.8×10¹², from 5×10⁷ to 1.8×10¹², from 6×10⁷ to 1.8×10¹², from 7×10⁷ to 1.8×10¹², from 8×10⁷ to 1.8×10¹², from 9×10⁷ to 1.8×10¹², from 1×10⁸ to 1.8×10¹², from 2×10⁸ to 1.8×10¹², from 3×10⁸ to 1.8×10¹², from 4×10⁸ to 1.8×10¹², from 5×10⁸ to 1.8×10¹², from 6×10⁸ to 1.8×10¹², from 7×10⁸ to 1.8×10¹², from 8×10⁸ to 1.8×10¹², from 9×10⁸ to 1.8×10¹², from 1×10⁹ to 1.8×10¹², from 2×10⁹ to 1.8×10¹², from 3×10⁹ to 1.8×10¹², from 4×10⁹ to 1.8×10¹², from 5×10⁹ to 1.8×10¹², from 6×10⁹ to 1.8×10¹², from 7×10⁹ to 1.8×10¹², from 8×10⁹ to 1.8×10¹², from 9×10⁹ to 1.8×10¹², from 1×10¹⁰ to 1.8×10¹², from 2×10¹⁰ to 1.8×10¹², from 3×10¹⁰ to 1.8×10¹², from 4×10¹⁰ to 1.8×10¹², from 5×10¹⁰ to 1.8×10¹², from 6×10¹⁰ to 1.8×10¹², from 7×10¹⁰ to 1.8×10¹², from 8×10¹⁰ to 1.8×10¹², from 9×10¹⁰ to 1.8×10¹², from 1×10¹¹ to 1.8×10¹², from 2×10¹¹ to 1.8×10¹², from 3×10¹¹ to 1.8×10¹², from 4×10¹¹ to 1.8×10¹², from 5×10¹¹ to 1.8×10¹², from 6×10¹¹ to 1.8×10¹², from 7×10¹¹ to 1.8×10¹², from 8×10¹¹ to 1.8×10¹², from 9×10¹¹ to 1.8×10¹², or from 1×10¹² to 1.8×10¹²) stromal cells.

### Aggregated Hepatocytes and Stromal Cells

The cellular compositions disclosed herein can be provided as a suspension in a biocompatible scaffold containing hepatocytes and optionally stromal cells (e.g., fibroblasts). In some embodiments, the population of hepatocytes and the optional population of stromal cells are aggregated in spheroids. For example, in some embodiments, the population of hepatocytes and the optional population of stromal cells are aggregated in spheroids and the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

In some embodiments, the hepatocytes and optional stromal cells are in a biocompatible scaffold, and the population of hepatocytes and the population of stromal cells together account for at least 70% (e.g., at least 71%, 72%, 73%, 74%, 75%, 80%, 85%, 90%, 95%, or 100%) of the total cells in the engineered tissue construct. For example, in some embodiments, the population of hepatocytes and the population of stromal cells together account for at least 71% of the total cells in the engineered tissue construct. In some embodiments, the population of hepatocytes and the population of stromal cells together account for at least 72% of the total cells in the engineered tissue construct. In some embodiments, the population of hepatocytes and the population of stromal cells together account for at least 73% of the total cells in the engineered tissue construct. In some embodiments, the population of hepatocytes and the population of stromal cells together account for at least 74% of the total cells in the engineered tissue construct. In some embodiments, the population of hepatocytes and the population of stromal cells together account for at least 75% of the total cells in the engineered tissue construct. In some embodiments, the population of hepatocytes and the population of stromal cells together account for at least 80% of the total cells in the engineered tissue construct. In some embodiments, the population of hepatocytes and the population of stromal cells together account for at least 85% of the total cells in the engineered tissue construct. In some embodiments, the population of hepatocytes and the population of stromal cells together account for at least 90% of the total cells in the engineered tissue construct. In some embodiments, the population of hepatocytes and the population of stromal cells together account for at least 95% of the total cells in the engineered tissue construct. In some embodiments, the population of hepatocytes and the population of stromal cells together account for 100% of the total cells in the engineered tissue construct.

In some embodiments, the hepatocytes and optional stromal cells are distributed non-homogenously along the z-axis of the biocompatible scaffold.

In some embodiments, the hepatocytes and optional stromal cells are distributed homogenously along the x-axis of the biocompatible scaffold.

In some embodiments, the hepatocytes and optional stromal cells are distributed homogenously along the y-axis of the biocompatible scaffold.

In some embodiments, the ratio of hepatocytes to stromal cells is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

For example, in some embodiments, the ratio of hepatocytes to stromal cells is between 1:9 and 4:1. In some embodiments, the ratio of hepatocytes to stromal cells is between 1:8 and 4:1. In some embodiments, the ratio of hepatocytes to stromal cells is between 1:7 and 4:1. In some embodiments, the ratio of hepatocytes to stromal cells is between 1:6 and 4:1. In some embodiments, the ratio of hepatocytes to stromal cells is between 1:5 and 4:1. In some embodiments, the ratio of hepatocytes to stromal cells is between 1:4 and 4:1. In some embodiments, the ratio of hepatocytes to stromal cells is between 1:3 and 4:1. In some embodiments, the ratio of hepatocytes to stromal cells is between 1:2 and 4:1. In some embodiments, the ratio of hepatocytes to stromal cells is between 1:1 and 4:1. In some embodiments, the ratio of hepatocytes to stromal cells is between 1:0 and 4:1.

In some embodiments, the ratio of hepatocytes to stromal cells is between 1:10 and 3:1. In some embodiments, the ratio of hepatocytes to stromal cells is between 1:9 and 3:1. In some embodiments, the ratio of hepatocytes to stromal cells is between 1:8 and 3:1. In some embodiments, the ratio of hepatocytes to stromal cells is between 1:7 and 3:1. In some embodiments, the ratio of hepatocytes to stromal cells is between 1:6 and 3:1. In some embodiments, the ratio of hepatocytes to stromal cells is between 1:5 and 3:1. In some embodiments, the ratio of hepatocytes to stromal cells is between 1:4 and 3:1. In some embodiments, the ratio of hepatocytes to stromal cells is between 1:3 and 3:1. In some embodiments, the ratio of hepatocytes to stromal cells is between 1:2 and 3:1. In some embodiments, the ratio of hepatocytes to stromal cells is between 1:1 and 3:1. In some embodiments, the ratio of hepatocytes to stromal cells is between 1:0 and 3:1.

In some embodiments, the ratio of hepatocytes to stromal cells is between 1:10 and 2:1. In some embodiments, the ratio of hepatocytes to stromal cells is between 1:9 and 2:1. In some embodiments, the ratio of hepatocytes to stromal cells is between 1:8 and 2:1. In some embodiments, the ratio of hepatocytes to stromal cells is between 1:7 and 2:1. In some embodiments, the ratio of hepatocytes to stromal cells is between 1:6 and 2:1. In some embodiments, the ratio of hepatocytes to stromal cells is between 1:5 and 2:1. In some embodiments, the ratio of hepatocytes to stromal cells is between 1:4 and 2:1. In some embodiments, the ratio of hepatocytes to stromal cells is between 1:3 and 2:1. In some embodiments, the ratio of hepatocytes to stromal cells is between 1:2 and 2:1. In some embodiments, the ratio of hepatocytes to stromal cells is between 1:1 and 2:1. In some embodiments, the ratio of hepatocytes to stromal cells is between 1:0 and 2:1.

In some embodiments, the ratio of hepatocytes to stromal cells is between 1:10 and 1:1. In some embodiments, the ratio of hepatocytes to stromal cells is between 1:9 and 1:1. In some embodiments, the ratio of hepatocytes to stromal cells is between 1:8 and 1:1. In some embodiments, the ratio of hepatocytes to stromal cells is between 1:7 and 1:1. In some embodiments, the ratio of hepatocytes to stromal cells is between 1:6 and 1:1. In some embodiments, the ratio of hepatocytes to stromal cells is between 1:5 and 1:1. In some embodiments, the ratio of hepatocytes to stromal cells is between 1:4 and 1:1. In some embodiments, the ratio of hepatocytes to stromal cells is between 1:3 and 1:1. In some embodiments, the ratio of hepatocytes to stromal cells is between 1:2 and 1:1. In some embodiments, the ratio of hepatocytes to stromal cells is between 1:1 and 1:0.

In some embodiments, the layer of hepatocyte and optional stromal cell aggregates in the biocompatible scaffold is from 100 μm to 1 mm (e.g., 200 μm to 900 μm, 300 μm to 800 μm, 400 μm to 700 μm, or 500 μm to 600 μm) thick. For example, in some embodiments, the layer of hepatocyte and stromal cell aggregates in the biocompatible scaffold is from 200 μm to 900 μm thick. In some embodiments, the layer of hepatocyte and stromal cell aggregates in the biocompatible scaffold is from 300 μm to 800 μm thick. In some embodiments, the layer of hepatocyte and stromal cell aggregates in the biocompatible scaffold is from 400 μm to 700 μm thick. In some embodiments, the layer of hepatocyte and stromal cell aggregates in the biocompatible scaffold is from 500 μm to 600 μm thick.

In some embodiments, the layer of hepatocyte and stromal cell aggregates in the biocompatible scaffold is 100 μm thick. In some embodiments, the layer of hepatocyte and stromal cell aggregates in the biocompatible scaffold is 200 μm thick. In some embodiments, the layer of hepatocyte and stromal cell aggregates in the biocompatible scaffold is 300 μm thick. In some embodiments, the layer of hepatocyte and stromal cell aggregates in the biocompatible scaffold is 400 μm thick. In some embodiments, the layer of hepatocyte and stromal cell aggregates in the biocompatible scaffold is 500 μm thick. In some embodiments, the layer of hepatocyte and stromal cell aggregates in the biocompatible scaffold is 600 μm thick. In some embodiments, the layer of hepatocyte and stromal cell aggregates in the biocompatible scaffold is 700 μm thick. In some embodiments, the layer of hepatocyte and stromal cell aggregates in the biocompatible scaffold is 800 μm thick. In some embodiments, the layer of hepatocyte and stromal cell aggregates in the biocompatible scaffold is 900 μm thick. In some embodiments, the layer of hepatocyte and stromal cell aggregates in the biocompatible scaffold is 1 mm thick. In some embodiments, the layer of hepatocyte and stromal cell aggregates in the biocompatible scaffold is 2 mm thick.

In some embodiments, the density of hepatocytes in the layer of hepatocyte and optional stromal cell aggregates in the biocompatible scaffold is from 0.06 M/cm² to 150 M/cm² (e.g., 0.07 M/cm² to 149 M/cm², 0.08 M/cm² to 148 M/cm², 0.09 M/cm² to 147 M/cm², 0.1 M/cm² to 146 M/cm², 0.2 M/cm² to 145 M/cm², 0.3 M/cm² to 140 M/cm², 0.4 M/cm² to 130 M/cm², 0.5 M/cm² to 120 M/cm², 1 M/cm² to 110 M/cm², 2 M/cm² to 100 M/cm², 3 M/cm² to 50 M/cm², 4 M/cm² to 40 M/cm², 5 M/cm² to 30 M/cm², or 10 M/cm² to 20 M/cm²). For example, in some embodiments, the density of hepatocytes in the layer of hepatocyte and stromal cell aggregates in the biocompatible scaffold is from 0.07 M/cm² to 149 M/cm². In some embodiments, the density of hepatocytes in the layer of hepatocyte and stromal cell aggregates in the biocompatible scaffold is from 0.08 M/cm² to 148 M/cm². In some embodiments, the density of hepatocytes in the layer of hepatocyte and stromal cell aggregates in the biocompatible scaffold is from 0.09 M/cm² to 147 M/cm². In some embodiments, the density of hepatocytes in the layer of hepatocyte and stromal cell aggregates in the biocompatible scaffold is from 0.1 M/cm² to 146 M/cm². In some embodiments, the density of hepatocytes in the layer of hepatocyte and stromal cell aggregates in the biocompatible scaffold is from 0.2 M/cm² to 145 M/cm². In some embodiments, the density of hepatocytes in the layer of hepatocyte and stromal cell aggregates in the biocompatible scaffold is from 0.3 M/cm² to 140 M/cm². In some embodiments, the density of hepatocytes in the layer of hepatocyte and stromal cell aggregates in the biocompatible scaffold is from 0.4 M/cm² to 130 M/cm². In some embodiments, the density of hepatocytes in the layer of hepatocyte and stromal cell aggregates in the biocompatible scaffold is from 0.5 M/cm² to 120 M/cm². In some embodiments, the density of hepatocytes in the layer of hepatocyte and stromal cell aggregates in the biocompatible scaffold is from 1 M/cm² to 110 M/cm². In some embodiments, the density of hepatocytes in the layer of hepatocyte and stromal cell aggregates in the biocompatible scaffold is from 2 M/cm² to 100 M/cm². In some embodiments, the density of hepatocytes in the layer of hepatocyte and stromal cell aggregates in the biocompatible scaffold is from 3 M/cm² to 50 M/cm². In some embodiments, the density of hepatocytes in the layer of hepatocyte and stromal cell aggregates in the biocompatible scaffold is from 4 M/cm² to 40 M/cm². In some embodiments, the density of hepatocytes in the layer of hepatocyte and stromal cell aggregates in the biocompatible scaffold is from 5 M/cm² to 30 M/cm². In some embodiments, the density of hepatocytes in the layer of hepatocyte and stromal cell aggregates in the biocompatible scaffold is from 10 M/cm² to 20 M/cm².

In some embodiments, the density of hepatocytes in the layer of hepatocyte and stromal cell aggregates in the biocompatible scaffold is 0.06 M/cm². In some embodiments, the density of hepatocytes in the layer of hepatocyte and stromal cell aggregates in the biocompatible scaffold is 0.07 M/cm². In some embodiments, the density of hepatocytes in the layer of hepatocyte and stromal cell aggregates in the biocompatible scaffold is 0.08 M/cm². In some embodiments, the density of hepatocytes in the layer of hepatocyte and stromal cell aggregates in the biocompatible scaffold is 0.09 M/cm². In some embodiments, the density of hepatocytes in the layer of hepatocyte and stromal cell aggregates in the biocompatible scaffold is 0.1 M/cm². In some embodiments, the density of hepatocytes in the layer of hepatocyte and stromal cell aggregates in the biocompatible scaffold is 0.2 M/cm². In some embodiments, the density of hepatocytes in the layer of hepatocyte and stromal cell aggregates in the biocompatible scaffold is 0.3 M/cm². In some embodiments, the density of hepatocytes in the layer of hepatocyte and stromal cell aggregates in the biocompatible scaffold is 0.4 M/cm². In some embodiments, the density of hepatocytes in the layer of hepatocyte and stromal cell aggregates in the biocompatible scaffold is 0.5 M/cm². In some embodiments, the density of hepatocytes in the layer of hepatocyte and stromal cell aggregates in the biocompatible scaffold is 1 M/cm². In some embodiments, the density of hepatocytes in the layer of hepatocyte and stromal cell aggregates in the biocompatible scaffold is 2 M/cm². In some embodiments, the density of hepatocytes in the layer of hepatocyte and stromal cell aggregates in the biocompatible scaffold is 3 M/cm². In some embodiments, the density of hepatocytes in the layer of hepatocyte and stromal cell aggregates in the biocompatible scaffold is 4 M/cm². In some embodiments, the density of hepatocytes in the layer of hepatocyte and stromal cell aggregates in the biocompatible scaffold is 5 M/cm². In some embodiments, the density of hepatocytes in the layer of hepatocyte and stromal cell aggregates in the biocompatible scaffold is 10 M/cm². In some embodiments, the density of hepatocytes in the layer of hepatocyte and stromal cell aggregates in the biocompatible scaffold is 20 M/cm². In some embodiments, the density of hepatocytes in the layer of hepatocyte and stromal cell aggregates in the biocompatible scaffold is 30 M/cm². In some embodiments, the density of hepatocytes in the layer of hepatocyte and stromal cell aggregates in the biocompatible scaffold is 40 M/cm². In some embodiments, the density of hepatocytes in the layer of hepatocyte and stromal cell aggregates in the biocompatible scaffold is 50 M/cm². In some embodiments, the density of hepatocytes in the layer of hepatocyte and stromal cell aggregates in the biocompatible scaffold is 100 M/cm². In some embodiments, the density of hepatocytes in the layer of hepatocyte and stromal cell aggregates in the biocompatible scaffold is 110 M/cm². In some embodiments, the density of hepatocytes in the layer of hepatocyte and stromal cell aggregates in the biocompatible scaffold is 120 M/cm². In some embodiments, the density of hepatocytes in the layer of hepatocyte and stromal cell aggregates in the biocompatible scaffold is 130 M/cm². In some embodiments, the density of hepatocytes in the layer of hepatocyte and stromal cell aggregates in the biocompatible scaffold is 140 M/cm². In some embodiments, the density of hepatocytes in the layer of hepatocyte and stromal cell aggregates in the biocompatible scaffold is 145 M/cm². In some embodiments, the density of hepatocytes in the layer of hepatocyte and stromal cell aggregates in the biocompatible scaffold is 146 M/cm². In some embodiments, the density of hepatocytes in the layer of hepatocyte and stromal cell aggregates in the biocompatible scaffold is 147 M/cm². In some embodiments, the density of hepatocytes in the layer of hepatocyte and stromal cell aggregates in the biocompatible scaffold is 148 M/cm². In some embodiments, the density of hepatocytes in the layer of hepatocyte and stromal cell aggregates in the biocompatible scaffold is 149 M/cm². In some embodiments, the density of hepatocytes in the layer of hepatocyte and stromal cell aggregates in the biocompatible scaffold is 150 M/cm².

In some embodiments, the thickness of the layer is dependent upon the hepatocyte-density.

### Other Cell Types

In some embodiments, the engineered tissue construct includes less than 30% (e.g., less than 29%, 28%, 27%, 26%, 25%, 20%, 15%, 10%, or 21%) of other cell types. For example, other cell types may include stellate cells, Kupffer cells, pit cells, cholangiocytes, portal fibroblasts, and liver sinusoidal endothelial cells. In some embodiments, the engineered tissue construct includes less than 30% (e.g., less than 29%, 28%, 27%, 26%, 25%, 20%, 15%, 10%, or 21%) of stellate cells. In some embodiments, the engineered tissue construct includes less than 30% (e.g., less than 29%, 28%, 27%, 26%, 25%, 20%, 15%, 10%, or 21%) of Kupffer cells. In some embodiments, the engineered tissue construct includes less than 30% (e.g., less than 29%, 28%, 27%, 26%, 25%, 20%, 15%, 10%, or 21%) of pit cells. In some embodiments, the engineered tissue construct includes less than 30% (e.g., less than 29%, 28%, 27%, 26%, 25%, 20%, 15%, 10%, or 21%) of bile duct cells. In some embodiments, the engineered tissue construct exclusively includes hepatocytes.

In some embodiments, the engineered tissue construct includes endothelial cells. In some embodiments, the engineered tissue construct includes up to 30% (e.g., up to 29%, 28%, 27%, 26%, 25%, 20%, 15%, 10%, or 1%) of endothelial cells. For example, in some embodiments, the engineered tissue construct includes up to 29% of endothelial cells. In some embodiments, the engineered tissue construct includes up to 28% of endothelial cells. In some embodiments, the engineered tissue construct includes up to 27% of endothelial cells. In some embodiments, the engineered tissue construct includes up to 26% of endothelial cells. In some embodiments, the engineered tissue construct includes up to 25% of endothelial cells. In some embodiments, the engineered tissue construct includes up to 20% of endothelial cells. In some embodiments, the engineered tissue construct includes up to 15% of endothelial cells. In some embodiments, the engineered tissue construct includes up to 10% of endothelial cells. In some embodiments, the engineered tissue construct includes up to 1% of endothelial cells. In some embodiments, the population of endothelial cells is arranged as one or more cords.

### Biocompatible Hydrogel Scaffolds

The cellular compositions disclosed herein can be provided as a suspension in a biocompatible scaffold containing the cells, e.g., hepatocytes and stromal cells. In some embodiments, the population of cells (e.g., the population of hepatocytes and the population of stromal cells) are aggregated in spheroids. In some embodiments, the biocompatible scaffold has an x-axis, a y-axis, and a z-axis. For example, in some embodiments, the population of cells, e.g., hepatocytes and the optional population of stromal cells, are aggregated in spheroids and the spheroids are distributed non-homogenously, in a layer, along the z-axis of the biocompatible scaffold. In some embodiments, the spheroids are distributed homogenously along the x-axis of the biocompatible scaffold. In some embodiments, the spheroids are distributed homogenously along the y-axis of the biocompatible scaffold.

The biocompatible scaffold may be liquid, gel, semi-solid, or solid at room temperature (e.g., 25° C.). The biocompatible scaffold may be biodegradable or non-biodegradable. In some embodiments, the scaffold is bioresorbable or bioreplaceable. Exemplary biocompatible scaffolds include polymers and hydrogels include collagen, fibrinogen, fibrin, chitosan, MATRIGEL™, dextrans including chemically cross-linkable or photo-cross-linkable dextrans, processed tissue matrix such as submucosal tissue, PEG hydrogels (e.g., heparin-conjugated PEG hydrogels), poly(lactic-co-glycolic acid) (PLGA), hydroxyethyl methacrylate (HEMA), gelatin, alginate, agarose, polysaccharides, hyaluronic acid (HA), peptide-based self-assembling gels, thermo-responsive poly(NIPAAm). A number of biopolymers are known to those skilled in the art (Bryant and Anseth, J. Biomed. Mater. Res. (2002) 59(1):63-72; Mann et al., Biomaterials (2001) 22 (22): 3045-3051; Mann et al., Biomaterials (2001) 22 (5):439-444, and Peppas et al., Eur. J. Pharm. Biopharm. (2000) 50(1), 27-46; all incorporated by reference). In other embodiments, the biocompatible scaffold may contain a biopolymer having any of a number of growth factors, adhesion molecules, degradation sites or bioactive agents to enhance cell viability or for any of a number of other reasons. Such molecules are well known to those skilled in the art.

In some embodiments, the PEG hydrogel may be chemically cross-linkable and/or modified with bifunctional groups.

In certain embodiments, the biocompatible scaffold includes allogeneic components, autologous components, or both allogeneic components and autologous components. In certain embodiments, the biocompatible scaffold includes synthetic or semi-synthetic materials. In certain embodiments, the biocompatible scaffold includes a framework or support, such as a fibrin-derived scaffold.

In some embodiments, the biocompatible scaffold is fibrin.

Biocompatible hydrogel scaffolds suitable for use include any polymer that is gellable in situ, e.g., one that does not require chemicals or conditions (e.g., temperature or pH) that are not cytocompatible. This includes both stable and biodegradable biopolymers.

Polymers for use herein are preferably crosslinked, for example, ionically crosslinked. In certain embodiments, the methods and constructs described herein use polymers in which polymerization can be promoted photochemically (i.e., photo crosslinked), by exposure to an appropriate wavelength of light (i.e., photopolymerizable) or a polymer which is weakened or rendered soluble by light exposure or other stimulus. Although some of the polymers listed above are not inherently light sensitive (e.g., collagen, HA), they may be made light sensitive by the addition of acrylate or other photosensitive groups.

In certain embodiments, the method utilizes a photoinitiator. A photoinitiator is a molecule that is capable of promoting polymerization of hydrogels upon exposure to an appropriate wavelength of light as defined by the reactive groups on the molecule. In the context of the disclosure, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-1propanone) are all activated with UV light (365 nm). Other crosslinking agents activated by wavelengths of light that are cytocompatible (e.g., blue light) can also be used with the methods described herein.

In other embodiments, the method involves the use of polymers bearing non-photochemically polymerizable moieties. In certain embodiments, the non-photochemically polymerizable moieties are Michael acceptors. Non-limiting examples of such Michael acceptor moieties include α,β-unsaturated ketones, esters, amides, sulfones, sulfoxides, phosphonates. Additional non-limiting examples of Michael acceptors include quinines and vinyl pyridines. In some embodiments, the polymerization of Michael acceptors is promoted by a nucleophile. Suitable nucleophiles include, but are not limited to thiols, amines, alcohols, and molecules possessing thiol, amine, and alcohol moieties. In certain embodiments, the disclosure features use of thermally crosslinked polymers.

In some embodiments, the z-axis of the biocompatible scaffold is from 500 μm to 5 mm (e.g., 600 μm to 4 mm, 700 μm to 3 mm, 800 μm to 2 mm, or 900 μm to 1 mm). For example, in some embodiments, the z-axis of the biocompatible scaffold is from 600 μm to 4 mm. In some embodiments, the z-axis of the biocompatible scaffold is from 700 μm to 3 mm. In some embodiments, the z-axis of the biocompatible scaffold is from 800 μm to 2 mm. In some embodiments, the z-axis of the biocompatible scaffold is from 900 μm to 1 mm.

In some embodiments, the z-axis of the biocompatible scaffold is 500 μm. In some embodiments, the z-axis of the biocompatible scaffold is 600 μm. In some embodiments, the z-axis of the biocompatible scaffold is 700 μm. In some embodiments, the z-axis of the biocompatible scaffold is 800 μm. In some embodiments, the z-axis of the biocompatible scaffold is 900 μm. In some embodiments, the z-axis of the biocompatible scaffold is 1 mm. In some embodiments, the z-axis of the biocompatible scaffold is 2 mm. In some embodiments, the z-axis of the biocompatible scaffold is 3 mm. In some embodiments, the z-axis of the biocompatible scaffold is 4 mm. In some embodiments, the z-axis of the biocompatible scaffold is 5 mm.

In some embodiments, the ratio of height of the biocompatible scaffold to height of the layer of cell aggregates, e.g., hepatocyte and stromal cell aggregates, in the biocompatible scaffold is from 20:1 to 1:1 (e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1). For example, in some embodiments the ratio of height of the biocompatible scaffold to height of the layer is from 19:1 to 1:1. In some embodiments, the ratio of height of the biocompatible scaffold to height of the layer is from 18:1 to 1:1. In some embodiments, the ratio of height of the biocompatible scaffold to height of the layer is from 17:1 to 1:1. In some embodiments, the ratio of height of the biocompatible scaffold to height of the layer is from 16:1 to 1:1. In some embodiments, the ratio of height of the biocompatible scaffold to height of the layer is from 15:1 to 1:1. In some embodiments, the ratio of height of the biocompatible scaffold to height of the layer is from 14:1 to 1:1. In some embodiments, the ratio of height of the biocompatible scaffold to height of the layer is from 13:1 to 1:1. In some embodiments, the ratio of height of the biocompatible scaffold to height of the layer is from 12:1 to 1:1. In some embodiments, the ratio of height of the biocompatible scaffold to height of the layer is from 11:1 to 1:1. In some embodiments, the ratio of height of the biocompatible scaffold to height of the layer is from 10:1 to 1:1. In some embodiments, the ratio of height of the biocompatible scaffold to height of the layer is from 9:1 to 1:1. In some embodiments, the ratio of height of the biocompatible scaffold to height of the layer is from 8:1 to 1:1. In some embodiments, the ratio of height of the biocompatible scaffold to height of the layer is from 7:1 to 1:1. In some embodiments, the ratio of height of the biocompatible scaffold to height of the layer is from 6:1 to 1:1. In some embodiments, the ratio of height of the biocompatible scaffold to height of the layer is from 5:1 to 1:1. In some embodiments, the ratio of height of the biocompatible scaffold to height of the layer is from 4:1 to 1:1. In some embodiments, the ratio of height of the biocompatible scaffold to height of the layer is from 3:1 to 1:1.

In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel comprising the synthetic polymer described herein can bind various bioactive agents, e.g., growth factors, thereby preventing the bioactive agents from diffusing away and maintaining the bioactive agents at a high concentration locally, so that they can act on cells and promote various cell functions.

### Methods for Making Engineered Tissue Constructs

The method for making an engineered tissue construct described herein involves mammalian (e.g., human) cells. The methods for making an engineered tissue construct described herein may involve one or more cell types. In some embodiments, the engineered tissue construct includes hepatocytes (e.g., primary human hepatocytes (PHH)) and optionally stromal cells (e.g., fibroblasts e.g., human dermal fibroblasts e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). Frozen master cell banks (MCB) were sourced through external suppliers and were received as cryopreserved cells. All cell types are terminally differentiated cells isolated from primary donors obtained with appropriate donor consent for therapeutic use. For example, hepatocytes (e.g., PHH) are obtained from cadaveric donors via collagenase perfusion, Percoll density gradient purification, and subsequent cryopreservation to create an MCB. Hepatocytes are stored cryopreserved until initiation of a manufacturing build. Prior to accepting the lot as a released MCB, release testing is conducted on hepatocyte (e.g., PHH) candidate MCBs to establish that their performance characteristics meet acceptance criteria for characterization, release, and stability. Stromal cells (e.g., fibroblasts e.g., human dermal fibroblasts e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts) are, for example, isolated from a single donor of neonatal foreskin by physical separation of dermal and epidermal layers and sequential digestion with dispase and collagenase. After isolation, stromal cells are minimally expanded and cryopreserved to create a frozen MCB. Frozen MCBs are shipped to the manufacturing site and stromal cells are expanded to create working cell banks (WCB), which are then cryopreserved until initiation of a manufacturing build. These WCB are released based on specific acceptance criteria prior to use in the manufacturing process.

Upon initiation of a manufacturing build, stromal cells (e.g., fibroblasts e.g., human dermal fibroblasts e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts) are thawed from their respective WCB expanded, and tested to measure viability and cell count. Hepatocytes (e.g., PHH) are thawed from the hepatocyte MCB and tested to measure viability and cell count prior to optionally being combined at a ratio (e.g., 1:2) with stromal cells, centrifuged into arrays of microwells (e.g., pyramidal microwells), and incubated for 2-3 days to promote self-assembly of the cells into multicellular hepatic aggregates (e.g., spheroidal aggregates). Hepatic aggregates (e.g., spheroidal aggregates) are deemed acceptable for encapsulation after microscopic confirmation of compaction.

The hepatocyte and optional stromal cell aggregates are then encapsulated with a solution (e.g., a fibrinogen solution) that is polymerized (e.g., with thrombin). These encapsulation steps occur within a mold (e.g., a cylindrical mold) that controls the overall dimensions of the engineered tissue construct to be 500 μm to 5 mm in thickness and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension and targeted to be 2 mm in thickness.

In some embodiments, the mold may be any shape (e.g., cylindrical, square, or square with rounded corners).

Within the solution (e.g., a fibrinogen solution that is polymerized e.g., with thrombin), the hepatocyte/stromal cell aggregates are allowed to non-homogenously distribute (e.g., by gravity) along the z-axis of the biocompatible scaffold into a layer (e.g., to settle), thereby forming a one-sided engineered tissue construct.

In some embodiments, two or more of the one-sided engineered tissue constructs are assembled with each of the layers facing outwardly, respectively, thereby forming a two-sided engineered tissue construct.

The engineered tissue constructs of the present disclosure can be formed by a process described herein. In some embodiments, engineered tissue constructs with defined cellular configurations in a biocompatible hydrogel scaffold may be prepared by photopatterning PEG hydrogels containing the hepatocyte and stromal cell populations, resulting in a hydrogel network consisting of 3D cell hepatocytes and stromal cells. Further control of cell orientation within these patterned domains may be achieved utilizing dielectrophoretic patterning techniques. Dielectrophoresis (DEP) can be used alone for patterning of cells in relatively homogeneous slabs of hydrogel or in conjunction with the photopolymerization method.

In some embodiments, organizing cells and material into spatial arrangements, such as engineered tissue constructs, can be accomplished by physically constraining the placement of cells/material by the use of wells or grooves, or injecting cells into microfluidic channels or oriented void spaces/pores. In certain embodiments, the cells can be organized by physically positioning cells with electric fields, magnetic tweezers, optical tweezers, ultrasound waves, pressure waves, or micromanipulators. In some embodiments, the population of hepatocytes and the population of stromal cells are aggregated in spheroids and the spheroids are allowed (e.g., by gravity) to non-homogenously distribute along the z-axis of the biocompatible scaffold into a layer

In certain embodiments, the method for fabricating engineered tissue constructs and embedding the constructs in extracellular matrix includes (1) generating 3D templates that have been defined with channels or trenches, (2) suspending the population of cells and the population of cells in liquid collagen and centrifuging these cells into the channels of the template, (3) removing excess cell/collagen suspension to allow aggregates to form, and (4) removing aggregates from templates via encapsulation in an extracellular matrix scaffold.

In some embodiments, the method for fabricating the engineered tissue constructs includes (1) suspending the population of cells in a naturally-derived and/or synthetic scaffolding, (2) placing the suspended cells into the channels of a 3D template, and (3) allowing the cells to form one or more aggregates at least partially embedded in the naturally-derived and/or synthetic scaffolding. In some embodiments, the 3D template can be generated by molding, templating, photolithography, printing, deposition, sacrificial molding, stereolithography, or a combination thereof.

In some embodiments, an engineered tissue construct can be fabricated through the use a custom 3D printer technology to extrude lattices of carbohydrate glass filaments with predefined diameters, spacings and orientations. For example, in some embodiments, soluble (clinical-grade, sterile) fibrinogen and thrombin are combined and poured over the lattice. After the solution has polymerized into insoluble fibrin, the carbohydrate filaments are dissolved, leaving behind channels within the fibrin. The channels can then be filled with a suspension of cells in a naturally derived or synthetic scaffolding (e.g., soluble type I collagen) that subsequently is polymerized to trap the cells within the channels.

The methods allow for the formation of three-dimensional scaffolds from hundreds of micrometers to tens of centimeters in length and width, and tens of micrometers to hundreds of micrometers in height. A resolution of up to 100 micrometers in the photopolymerization method and possible single cell resolution (10 μm) in the DEP method is achievable. Photopolymerization apparatus, DEP apparatus, and other methods to produce 3-dimensional co-cultures are described in U.S. Pat. No. 8,906,684, which is incorporated herein by reference.

The cells can be cultured in vitro under various culture conditions. The cells (e.g., primary cells) can be expanded in culture, e.g., grown under conditions that promote their proliferation. Culture medium can be liquid or semi-solid, e.g., containing agar, methylcellulose, and the like. The cell population can be suspended in an appropriate nutrient medium, such as Iscoves modified DMEM or RPMI 1640, normally supplemented with fetal calf serum (5-10%), L-glutamine, a thiol, particularly 2-mercaptoethanol, and antibiotics, e.g., penicillin and streptomycin. The culture can contain growth factors to which the regulatory T cells are responsive. Growth factors, as defined herein, can be molecules capable of promoting survival, growth and/or differentiation of cells, either in culture or in the intact tissue, through specific effects on a transmembrane receptor. Growth factors include polypeptides and non-polypeptide factors.

The cells produced by the methods described herein can be used immediately in the making of an engineered tissue construct. Alternatively, the cells can be frozen at liquid nitrogen temperatures and stored for long periods of time, being thawed and capable of being reused. For example, the cells can be frozen in 10% dimethylsulfoxide (DMSO), 50% serum, 40% buffered medium, or some other such solution as is commonly used in the art to preserve cells at such freezing temperatures and thawed in a manner as commonly known in the art for thawing frozen cultured cells.

### Methods of Treatment

The engineered tissue constructs described herein may be used to treat a disease or disorder in a subject, e.g., a human subject in need thereof. An engineered tissue construct containing a population of mammalian (e.g., human) cells that is implanted into a human subject may be used to treat, for example, a liver disease, diabetes, an endocrine disorder, a hormonal deficiency, a protein deficiency, impaired biotransformation, or a disease of impaired protein synthesis.

An engineered tissue construct may be implanted into a human subject to treat a disease or disorder associated with the liver. The liver disease may be, for example, acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4 (Andersen), hepatic encephalopathy, Hunter syndrome (MPS II), infantile refsum disease, lysosomal acid lipase deficiency (LAL-D) (cholesteryl ester storage disease), maple syrup urine disease, Maroteaux-Lamy (MPS VI), methylmalonic acidemia, ornithine transcarbamylase (OTC) deficiency, propionic acidemia, a urea cycle disorder, alpha-1 antitrypsin deficiency, Niemann-Pick type A/B, Niemann-Pick type C, primary hyperoxaluria type 1, primary hyperoxaluria type 2, primary hyperoxaluria type 3, pyruvate kinase deficiency, liver type, Wilsons disease, D-bifunctional protein deficiency, Gauchers disease, Hurler syndrome (MPS I), hypophosphatasia, morquio A (MPS 4A), morquio B (MPS 4B), multiple acyl-coa dehydrogenase deficiency, sanfilippo (MPS III), acute hepatic porphyrias (AHP), glycogen storage disorder type 3 (Cori), hereditary angioedema (HAE), hereditary hemochromatosis, homocystinuria cystathionine B-synthase deficiency, isovaleric acidemia, N-acetyglutamate synthetase deficiency (NAGS), pseudoxanthoma elasticum, tyrosinemia, 3-methylcrotonyl-CoA carboxylase deficiency (3-MCC), acute fatty liver of pregnancy, congenital factor V deficiency, congenital factor XI deficiency, congenital fibrinogen disorder, corticosteroid-binding globulin deficiency, cutaneous hepatic porphyrias, Fabry disease, factor VII deficiency, factor X deficiency, familial dysbetalipoproteinemia, familial hypertriglyceridemia, familial lipoprotein lipase deficiency, galactosemia, glutaric acidemia type 1, glycogen storage disorder type 1b, glycogen storage disorder type 6 (Hers), hemophilia type A (factor VIII deficiency), hemophilia type B (factor IX), hereditary fructose intolerance, long-chain L-3 hydroxyacyl-CoA dehydrogenase deficiency (LCHAD) deficiency, very long-chain acyl-CoA dehydrogenase (VLCAD) deficiency, 3-hydroxy-3-methylglutaryl-CoA synthase deficiency, abetalipoproteinemia, acetyl-CoA acetyltransferase-2 deficiency, adenosine kinase deficiency, adult polyglucosan body disease, delta-aminolevulinic acid (ALA) dehydratase (ALAD)-deficiency porphyria, alpha-2-plasmin inhibitor deficiency, aminolaevulinic acid dehydratase deficiency porphyria, atransferrinemia, beta-ketothiolase deficiency, bile acid CoA ligase deficiency and defective amidation, carboxypeptidase N deficiency, cerebral creatine deficiency syndrome 1, cerebral creatine deficiency syndrome 2, cerebral creatine deficiency syndrome 3, Chanarin-Dorfman syndrome, cirrhosis-dystonia-polycythemia-hypermanganesemia syndrome, combined oxidative phosphorylation deficiency 1, congenital disorder of deglycosylation, carnitine palmitoyltransferase (CPT) Deficiency, hepatic, type Ia, deoxyguanosine kinase deficiency, formiminoglutamic aciduria, gamma-glutamylcysteine synthetase deficiency, hepatic lipase deficiency, hepatic tuberculosis, Indian childhood cirrhosis, infantile liver failure syndrome, Lucey-Driscoll syndrome, mitochondrial DNA depletion syndrome, tangier disease, trifunctional protein deficiency, 3-hydroxyacyl-coenzyme A dehydrogenase deficiency, acyl-CoA oxidase deficiency, 3-hydroxy-3-methylglutaric aciduria, 2-methylbutyryl-coa dehydrogenase deficiency, acatalasemia, acquired fructose intolerance, cerebrotendinous xanthomatosis, conjugated hyperbilirubinemia (rotor syndrome), cystic echinococcosis, drug-induced hepatitis, Dubin-Johnson syndrome, focal fatty liver, Gilbert syndrome, glycine n-methyltransferase deficiency, hepatitis A, hepatitis E, liver abscess, liver fibrosis, nodular regenerative hyperplasia, nonalcoholic fatty liver disease (NAFLD), peliosis hepatis, phenylketonuria, short-chain acyl-CoA dehydrogenase deficiency (SCAD), trimethylaminuria, visceral steatosis, vitamin k-dependent clotting factors, combined deficiency of, type 1 and type 2, acute cholangitis/biliary tract infection, alagille syndrome, alphavirus infection, alveolar hydatid disease, benign postoperative, cholestasis, benign recurrent intrahepatic cholestasis, bile acid malabsorption, primary, bile duct cysts, Budd-Chiari syndrome, Caroli disease, cholestasis, clonorchiasis, congenital disorders of glycosylation, congenital hepatic fibrosis, erythropoietic protoporphyrias, familial amyloidosis, familial hypercholanemia, flavivirus infection, hepatic infarction, hepatic veno-occlusive disease, hepatolithiasis, hepatoportal sclerosis, hereditary hemorrhagic telangiectasia, IgG4-related sclerosing cholangitis, intrahepatic cholestasis, isolated neonatal sclerosing cholangitis, non-cirrhotic portal fibrosis, opisthorchiasis, polycystic liver disease, portal hypertension, portal vein thrombosis, primary biliary cholangitis, primary sclerosing cholangitis, progressive familial intrahepatic cholestasis, Reye syndrome, Reynolds syndrome, spontaneous bacterial peritonitis, Von Willebrand disease type 3, biliary atresia, biliary dyskinesia, biliary reflux, cholecystitis, cholelithiasis, alcoholic hepatitis, alcoholic liver disease, autoimmune hepatitis, cystic fibrosis liver disease, hepatitis D, hepatotoxicity, IgG4-related hepatopathy, liver cirrhosis, nonalcoholic steatohepatitis (NASH), hyperammonemia, TIPS-induced hyperammonemia, or small for size syndrome.

In some embodiments, the liver disease is acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4 (Andersen), hepatic encephalopathy, Hunter syndrome (MPS II), infantile refsum disease, lysosomal acid lipase deficiency (LAL-D) (cholesteryl ester storage disease), maple syrup urine disease, Maroteaux-Lamy (MPS VI), methylmalonic acidemia, ornithine transcarbamylase (OTC) deficiency, propionic acidemia, or a urea cycle disorder.

In some embodiments, the liver disease is acute liver failure, a urea cycle disorder, or Crigler-Najjar syndrome.

Other diseases and disorders may also be treated. For example, in some embodiments, any disease or disorder that may be treated by implantation of one or more cell types as disclosed herein may be treated.

For example, in some embodiments, the engineered tissue construct includes pancreatic cells, and the disease is a pancreas-associated disease. For example, in some embodiments, the disease is diabetes (e.g., type 1 diabetes or type 2 diabetes) or pancreatitis. In some examples, the disease is type 1 diabetes. For example, for treatment of type 1 diabetes or pancreatitis, the method may include implanting one or more engineered tissue constructs that include pancreatic cells (e.g., alpha, beta, gamma, delta, epsilon cells, or any combination thereof) at any of the implantation sites described herein. In some embodiments, the pancreatic cells comprise beta cells.

In another example, the engineered tissue construct comprises adrenal cells, and the disease is an adrenal-associated disease (e.g., Addisons syndrome, hypercortisolism, or Cushings syndrome).

In another example, the engineered tissue construct comprises parathyroid cells, and the disease is a parathyroid-associated disease (e.g., hypoparathyroidism).

In another example, the engineered tissue construct comprises thyroid cells, and the disease is a thyroid-associated disease (e.g., hypothyroidism or hyperthyroidism).

In another example, the engineered tissue construct comprises ovarian cells, and the disease is an ovarian-associated disease.

In another example, the engineered tissue construct comprises enteroendocrine cells (e.g., L cells and/or chromaffin cells), and the disease is an enteroendocrine cell-associated disease.

### Acute Liver Failure

Acute liver failure (ALF) is the appearance of severe complications after the first signs of liver disease (e.g., jaundice) in a patient (e.g., a human patient). ALF includes a number of conditions, most of which involve severe hepatocyte injury or necrosis. In most cases of ALF, massive necrosis of hepatocytes occurs. Altered mental status (hepatic encephalopathy) and coagulopathy in the setting of a hepatic disease generally define ALF. Consequently, ALF is generally clinically defined as the development of coagulopathy, usually an international normalized ratio (a measure of the time it takes blood to clot compared to an average value-INR) of greater than 1.5 (e.g., 2, 2.5, 3, 4, or 5), and any degree of mental alteration (encephalopathy) in a patient without preexisting cirrhosis and with an illness of less than 26 weeks (e.g., less than 25 weeks, less than 24, weeks, less than 23 weeks, less than 22 weeks, less than 21 weeks, and less than 20 weeks) duration. ALF indicates that the liver has sustained severe damage resulting in the dysfunction of 80-90% of liver cells.

ALF occurs when the liver fails rapidly. Hyperacute liver failure is characterized as failure of the liver within one week. ALF is characterized as the failure of the liver within 8-28 days. Subacute liver failure is characterized as the failure of the liver within 4-12 weeks.

In some embodiments, the human subject having ALF weighs less than 15 kg (e.g., less than 15 kg, less than 14 kg, less than 13, kg, less than 12 kg, less than 11 kg, less than 10 kg, less than 9 kg, less than 8 kg, less than 7 kg, less than 6 kg, less than 5 kg, less than 4 kg, or less than 3 kg). For example, in some embodiments, the human subject weighs less than 14 kg. In some embodiments, the human subject weighs less than 13 kg. In some embodiments, the human subject weighs less than 12 kg. In some embodiments, the human subject weighs less than 11 kg. In some embodiments, the human subject weighs less than 10 kg. In some embodiments, the human subject weighs less than 9 kg. In some embodiments, the human subject weighs less than 8 kg. In some embodiments, the human subject weighs less than 7 kg. In some embodiments, the human subject weighs less than 6 kg. In some embodiments, the human subject weighs less than 5 kg. In some embodiments, the human subject weighs less than 4 kg. In some embodiments, the human subject weighs less than 3 kg.

In some embodiments, the human subject having ALF weighs 15 kg. In some embodiments, the human subject weighs 14 kg. In some embodiments, the human subject weighs 13 kg. In some embodiments, the human subject weighs 12 kg. In some embodiments, the human subject weighs 11 kg. In some embodiments, the human subject weighs 10 kg. In some embodiments, the human subject weighs 9 kg. In some embodiments, the human subject weighs 8 kg. In some embodiments, the human subject weighs 7 kg. In some embodiments, the human subject weighs 6 kg. In some embodiments, the human subject weighs 5 kg. In some embodiments, the human subject weighs 4 kg. In some embodiments, the human subject weighs 3 kg.

In some embodiments, the human subject having ALF may be any weight.

In some embodiments, the method includes treating a subject having ALF, the method including implanting an engineered tissue construct including a population of hepatocytes and, optionally, a population of stromal cells. The implantation may be in any of the implantation sites described herein, e.g., in an extraperitoneal space, in an extrapleural space, or on a liver surface.

### Urea Cycle Disorders

The urea cycle is a cycle of biochemical reactions that produces urea from ammonia, a product of protein catabolism. The urea cycle includes five key enzymes including carbamoyl phosphate synthetase 1 (CPS1), ornithine transcarbamoylase (OTC), argininosuccinate synthetase (ASS1), argininosuccinate lyase (ASL), and arginase 1 (ARG1), but also requires other enzymes, such as N-acetylglutamate synthetase (NAGS), and mitochondrial amino acid transporters, such as ornithine translocase (ORNT1) and citrin. The urea cycle mainly occurs in the mitochondria of liver cells. The urea produced by the liver enters the bloodstream where it travels to the kidneys and is ultimately excreted in urine. Genetic defects in any of the enzymes or transporters in the urea cycle can cause a urea cycle or a symptom thereof.

In some embodiments, the method includes treating a subject having a urea cycle disorder, the method including implanting an engineered tissue construct including a population of hepatocytes and, optionally, a population of stromal cells. The implantation may be in any of the implantation sites described herein, e.g., in an extraperitoneal space, in an extrapleural space, or on a liver surface.

### Crigler-Najjar Syndromes

Hyperbilirubinemia is a condition in which there is an accumulation of bilirubin in the blood and serum bile acids appear to remain normal. A subpopulation of subjects experiencing hyperbilirubinemia have Crigler-Najjar syndromes (e.g., Crigler-Najjar type I and Crigler-Najjar type II). Subjects with Crigler-Najjar type I syndrome have total serum bilirubin levels exceeding 20 mg/dL. Their bile is almost completely composed of unconjugated bilirubin, with low levels of mono-glucuronide bilirubin. Subjects with Crigler-Najjar type II syndrome have total serum bilirubin levels in the range of 3.5-20 mg/dL.

In some embodiments, the method includes treating a subject having hyperbilirubinemia, the method including implanting an engineered tissue construct including a population of hepatocytes and, optionally, a population of stromal cells.

In some embodiments, the method includes treating a subject having Crigler-Najjar syndrome, the method including implanting an engineered tissue construct including a population of hepatocytes and, optionally, a population of stromal cells.

In some embodiments, the Crigler-Najjar syndrome is Crigler-Najjar syndrome type I. In some embodiments, the Crigler-Najjar syndrome is Crigler-Najjar syndrome type II.

In any of the preceding embodiments, the implantation may be in any of the implantation sites described herein, e.g., in an extraperitoneal space, in an extrapleural space, or on a liver surface.

### Recommended Clinical Parameters for Monitoring Following Implantation of the Engineered Tissue Construct

Following implantation of the engineered tissue construct, the subject may exhibit a change in one or more clinical parameters. For example, in some embodiments, following implantation of the engineered tissue construct, the subject exhibits a level of serum ammonia in the age-adjusted norm of less than or equal to 80 μmol/L (e.g., less than 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L). Alternatively, for example, in some embodiments, following implantation of the engineered tissue construct, the subject exhibits a level of serum ammonia of less than or equal to 500 μmol/L (e.g., less than 499 μmol/L, 488 μmol/L, 487 μmol/L, 486 μmol/L, 485 μmol/L, 480 μmol/L, 470 μmol/L, 460 μmol/L, 450 μmol/L, 400 μmol/L, 300 μmol/L, 200 μmol/L, 100 μmol/L, 50 μmol/L, 40 μmol/L, 30 μmol/L, 20 μmol/L, or 10 μmol/L).

In some embodiments, following implantation of the engineered tissue construct, the subject exhibits an improvement in a test of gallbladder ejection fraction (e.g., a hepatobiliary iminodiacetic acid scan). As yet another example, in some embodiments, following implantation of the engineered tissue construct, the subject exhibits a change in one or more parameters in a blood test relative to a reference level.

### Blood Test

Following implantation of the engineered tissue construct, the subject may exhibit a change in one or more parameters in a blood test (e.g., a liver function test (LFT), an ammonia test, or a bilirubin test). In some embodiments, the subject exhibits a change in one or more parameters (e.g., albumin, gamma-glutamyl transferase (GGT) level, alkaline phosphatase (ASP) level, aspartate aminotransferase (AST) level, alanine aminotransferase (ALT level), ammonia, or bilirubin level) in a blood test relative to a reference level following implantation of the engineered tissue construct.

### Albumin

In some embodiments, following implantation of the engineered tissue construct, the subject exhibits a change in the level of albumin, which can be measured with an LFT. For example, in some embodiments, following implantation of the engineered tissue construct, the subject exhibits a change in the level of albumin, such that their albumin level is returned to the age-adjusted norm. For example, if the subject was a human toddler (e.g., 6-12 months old), it would be determined that a subject exhibits a albumin level that is returned to the age-adjusted norm when the subjects albumin level is within the normal range of 30-55 U/L (e.g., 31-55 U/L, 32-55 U/L, 33-55 U/L, 34-55 U/L, 35-55 U/L, 36-55 U/L, 37-55 U/L, 38-55 U/L, 39-55 U/L, 40-55 U/L, 41-55 U/L, 42-55 U/L, 43-55 U/L, 44-55 U/L, 45-55 U/L, 46-55 U/L, 47-55 U/L, 48-55 U/L, 49-55 U/L, 50-55 U/L, 51-55 U/L, 52-55 U/L, 53-55 U/L, or 54-55 U/L).

Alternatively, for example, if the subject was a human aged 1-45 years old, it would be determined that a subject exhibits a albumin level that is returned to the age-adjusted norm when the subjects albumin level is within the normal range of 40-50 U/L (e.g., 41-50 U/L, 42-50 U/L, 43-50 U/L, 44-50 U/L, 45-50 U/L, 46-50 U/L, 47-50 U/L, 48-50 U/L, or 49-50 U/L).

If the subject was a human aged 46-90 years old, it would be determined that a subject exhibits an albumin level that is returned to the age-adjusted norm when the subjects albumin level is within the normal range of 35-50 U/L (e.g., 36-50 U/L, 37-50 U/L, 38-50 U/L, 39-50 U/L, 40-50 U/L, 41-50 U/L, 42-50 U/L, 43-50 U/L, 44-50 U/L, 45-50 U/L, 46-50 U/L, 47-50 U/L, 48-50 U/L, or 49-50 U/L).

### Gamma-Glutamyl Transferase

In some embodiments, following implantation of the engineered tissue construct, the subject exhibits a change in the level of GGT, which can be measured with an LFT. For example, in some embodiments, following implantation of the engineered tissue construct, the subject exhibits a change in the level of GGT, such that their GGT level is returned to the age-adjusted norm.

For example, if the subject was a human toddler (e.g., 6-12 months old), it would be determined that a subject exhibits a GGT level that is returned to the age-adjusted norm when the subjects GGT level is within the normal range of 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

Alternatively, for example, if the subject was a human child aged 1-5 years old, it would be determined that a subject exhibits a GGT level that is returned to the age-adjusted norm when the subjects GGT level is within the normal range of 3-22 U/L (e.g., 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

### Alkaline Phosphatase

In some embodiments, following implantation of the engineered tissue construct, the subject exhibits a change in the level of ASP, which can be measured with an LFT. For example, in some embodiments, following implantation of the engineered tissue construct, the subject exhibits a change in the level of ASP, such that their ASP level is returned to the age-adjusted norm.

For example, if the subject was a human, it would be determined that a subject exhibits an ASP level that is returned to the age-adjusted norm when the subjects ASP level is within the normal range of 50 to 300 U/L e.g., 51 to 300 U/L, 52 to U/L, 53 to 300 U/L, 54 to 300 U/L, 55 to 300 U/L, 56 to 300 U/L, 57 to 300 U/L, 58 to 300 U/L, 59 to 300 U/L, 60 to 300 U/L, 65 to 300 U/L, 70 to 300 U/L, 80 to 300 U/L, 90 to 300 U/L, 100 to 300 U/L, 125 to 300 U/L, 150 to 300 U/L, 175 to 300 U/L, 200 to 300 U/L, 225 to 300 U/L, 250 to 300 U/L, or 275 to 300 U/L).

### Aspartate Aminotransferase

In some embodiments, following implantation of the engineered tissue construct, the subject exhibits a change in the level of AST, which can be measured with an LFT. For example, in some embodiments, following implantation of the engineered tissue construct, the subject exhibits a change in the level of AST, such that their AST level is returned to the age-adjusted norm.

For example, if the subject was a human, it would be determined that a subject exhibits a AST level that is returned to the age-adjusted norm when the subjects AST level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, following implantation of the engineered tissue construct, the subject exhibits a change in the level of ALT, which can be measured with an LFT. For example, in some embodiments, following implantation of the engineered tissue construct, the subject exhibits a change in the level of ALT, such that their ALT level is returned to the age-adjusted norm.

For example, if the subject was a human, it would be determined that a subject exhibits a ALT level that is returned to the age-adjusted norm when the subjects ALT level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Bilirubin

In some embodiments, following implantation of the engineered tissue construct, the subject exhibits a change in the level of total bilirubin, which can be measured with a bilirubin test. For example, in some embodiments, following implantation of the engineered tissue construct, the subject exhibits a change in the level of total bilirubin, such that their total bilirubin level is returned to the age-adjusted norm of less than 1.2 mg/dL (e.g., less than 1.2 mg/dL, 1.1 mg/dL, 1 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL), e.g., for an adult. In other examples, following implantation of the engineered tissue construct, the subject exhibits a change in the level of total bilirubin, such that their total bilirubin level is returned to the age-adjusted norm of less than 1.0 mg/dL (e.g., less than 1 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL), e.g., for a subject under 18 years of age. In other embodiments, a reference range for total bilirubin for adults may be between 0.3 to 1.0 mg/dL.

In some embodiments, following implantation of the engineered tissue construct, the subject exhibits a change in the level of direct bilirubin, which can be measured with a bilirubin test. For example, in some embodiments, following implantation of the engineered tissue construct, the subject exhibits a change in the level of direct bilirubin, such that their direct bilirubin level is returned to the age-adjusted norm of less than −0.3 mg/dL (e.g., less than 0.3 mg/dL, 0.2 mg/dL, or 0.1 mg/dL). In other embodiments, a reference range for direct bilirubin for adults may be between 0.1 to 0.3 mg/dL.

In some embodiments, following implantation of the engineered tissue construct, the subject exhibits a change in the level of indirect bilirubin, which can be measured with a bilirubin test. For example, in some embodiments, following implantation of the engineered tissue construct, the subject exhibits a change in the level of indirect bilirubin, such that their bilirubin level is returned to the age-adjusted norm of less than 1 mg/dL (less than 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL). In other embodiments, a reference range for indirect bilirubin for adults may be between 0.2 to 0.7 mg/dL.

### Kits

The compositions described herein can be provided in a kit. The kit can include a package insert that instructs a user of the kit, such as a physician, to implant the engineered tissue construct. The kit may optionally include surgical equipment or another device for implanting the engineered tissue construct. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Engineered Tissue Construct Manufacturing

Engineered tissue constructs were manufactured with primary adult human hepatocytes (PHH) and neonatal human dermal fibroblasts (NHDF). Frozen master cell banks were sourced through external suppliers and were received as cryopreserved cells. All cell types are terminally differentiated cells isolated from primary donors obtained with appropriate donor consent for therapeutic use. PHH are obtained from cadaveric donors via collagenase perfusion, Percoll density gradient purification, and subsequent cryopreservation to create a master cell bank (MCB). Hepatocytes are stored cryopreserved until initiation of a manufacturing build. Prior to accepting the lot as a released MCB, release testing is conducted on PHH candidate MCBs to establish that their performance characteristics meet acceptance criteria for characterization, release and stability. NHDF are isolated from a single donor of neonatal foreskin by physical separation of dermal and epidermal layers and sequential digestion with dispase and collagenase. After isolation, NHDF are minimally expanded and cryopreserved to create a frozen MCB. Frozen MCBs are shipped to Satellite Bio and NHDF are expanded to create working cell banks (WCB) and then cryopreserved until initiation of a manufacturing build. These WCB are released based on specific acceptance criteria prior to use in the manufacturing process.

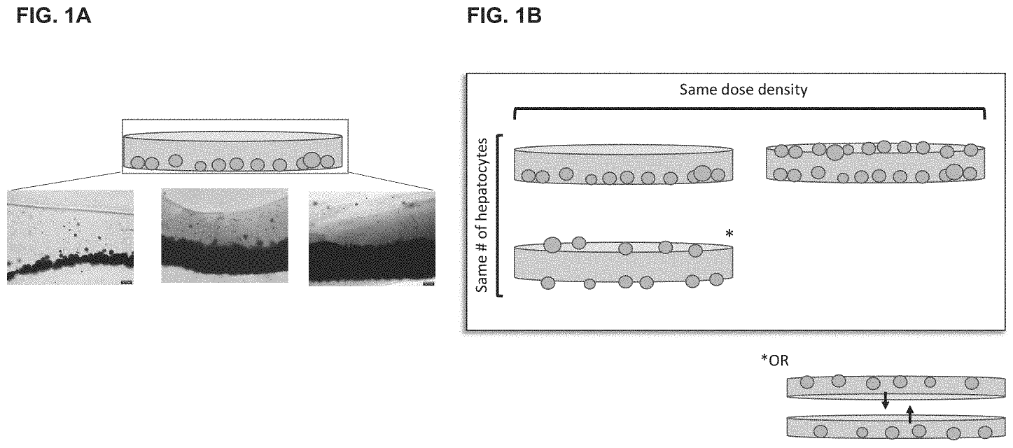

Upon initiation of a manufacturing build, NHDF are thawed from their respective WCB, expanded and tested to measure viability and cell count. PHH are thawed from the PHH MCB and tested to measure viability and cell count prior to being combined at a 1:2 ratio with NHDF, centrifuged into arrays of pyramidal microwells and incubated for 2-3 days to promote self-assembly of the cells into multicellular hepatic aggregates. Hepatic aggregates are deemed acceptable for encapsulation after microscopic confirmation of compaction (FIG. 1A).

The hepatocyte/stromal cell aggregates are then encapsulated with a fibrinogen solution that is polymerized with thrombin. These encapsulation steps occur within a cylindrical mold that controls the overall dimensions of the graft to be 2 mm in thickness and an outer diameter of 6 mm 200 mm. The thickness is controlled by the volume of cell-hydrogel suspension and targets 2 mm in thickness.

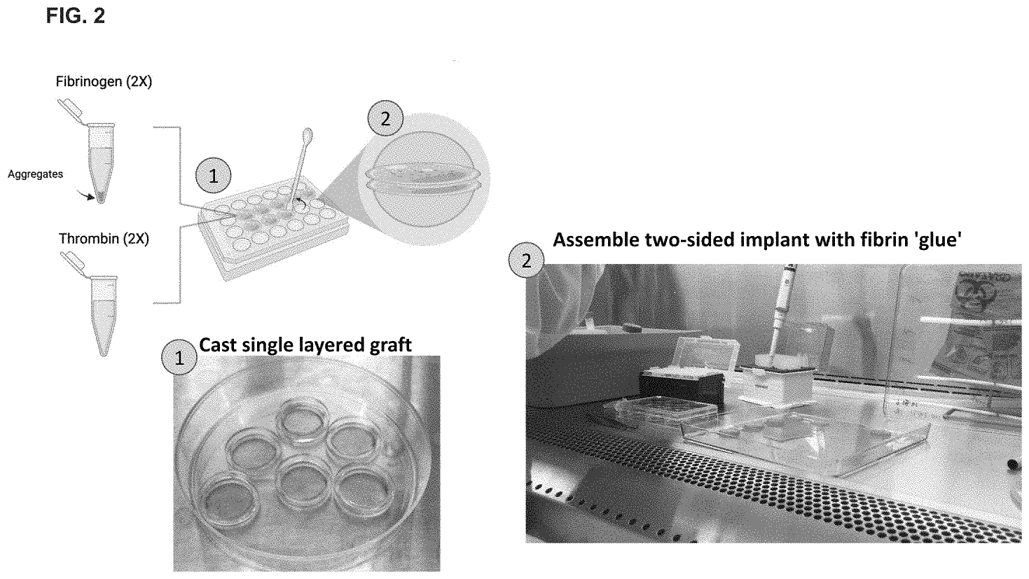

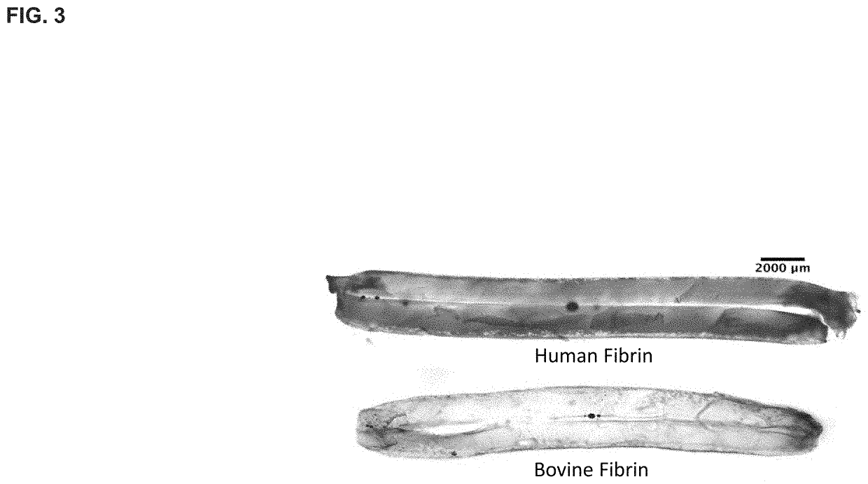

Furthermore, simultaneous with the polymerization step, the aggregates are allowed to distribute nonhomogeneously (e.g., via gravity into a layer at the bottom). Afterwards, one or more engineered tissue constructs may be assembled with biocompatible glue (e.g., fibrin glue) such that each of the aggregate layers is outward facing, thereby forming a two-sided engineered tissue construct (FIG. 1B, FIG. 2, and FIG. 3).

### Example 2. In Vivo Evaluation of Engineered Tissue Constructs in Two Immunodeficient Mouse Models

To determine the effectiveness of engineered tissue constructs in an in vivo model of acute liver failure, a study was performed with NOD-scid IL2Rgamma^{null} (NSG™) mice (4-8 weeks of age upon arrival). NSG™ mice are immunodeficient mice.

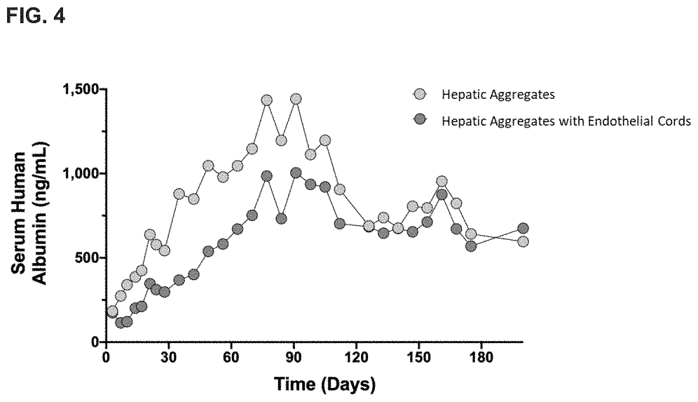

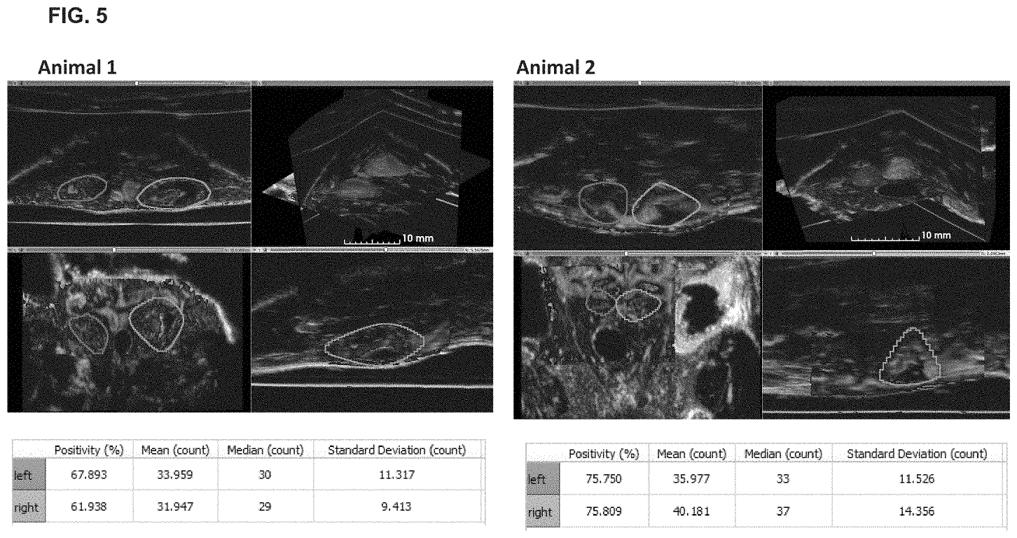

On day 0 of the study, NSG™ mice were implanted with two engineered tissue constructs, each consisting of 1.41×10⁶ PHH and 2.82×10⁶ NHDFS, with or without endothelial cells. Over time, mice implanted with engineered tissue constructs without endothelial cells (“Hepatic Aggregates”) showed elevated levels of human albumin (ng/mL), as compared to the experimental group with endothelial cells (“Hepatic Aggregates with Endothelial Cords”) (FIG. 4). The beneficial effect of engineered tissue constructs lacking endothelial cells was also apparent in the vascular volume of the implantation site, using SonoVol imaging (FIG. 5).

A second study was performed with transgenic thymidine kindase-NOD/Shi-scid/IL-2Rγ^{null} (TK-NOG) (Taconic model #12907-F) mice (4-8 weeks of age upon arrival). TK-NOG mice are immunodeficient mice with transgenic expression of thymidine kinase (TK) under control of a liver-restricted albumin promoter, which provide inducible ablation of hepatocytes by ganciclovir (GCV) treatment.

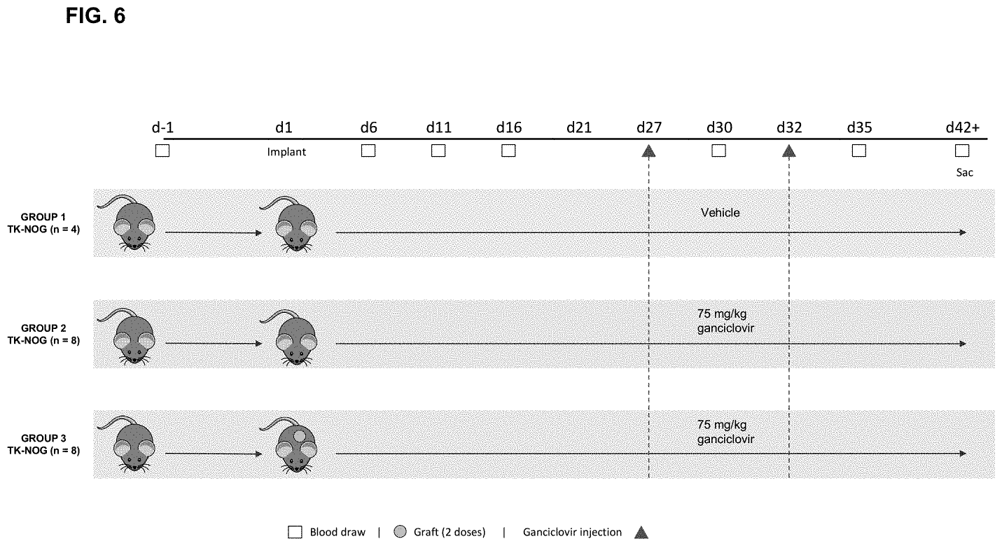

On day −1, each TK-NOG mouse underwent a blood draw. On day 1 mice were implanted with two engineered tissue constructs, including 0.7×1.06 PHH/mouse (low dose “group 3”) or 7×1.06 PHH/mouse (high dose “group 3). Following implantation, all mice underwent a blood draw on days 6, 11, 16, 21, 30, 35, and 42; as well as a dosing of GCV on days 27 and 32 (control group 2 and experimental group 3). Mice were sacrificed on day 42. Clinical observations were made daily, and blood was drawn with chemistry to determine the levels of liver enzymes performed on the blood drawn. Furthermore, GCV or phosphate buffered saline (PBS) was administered intraperitoneally (i.p.) (FIG. 6).

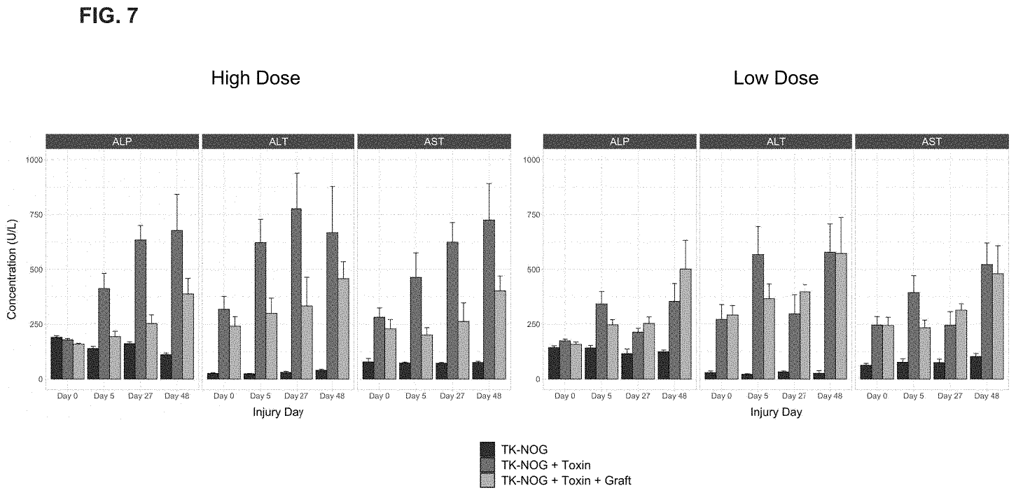

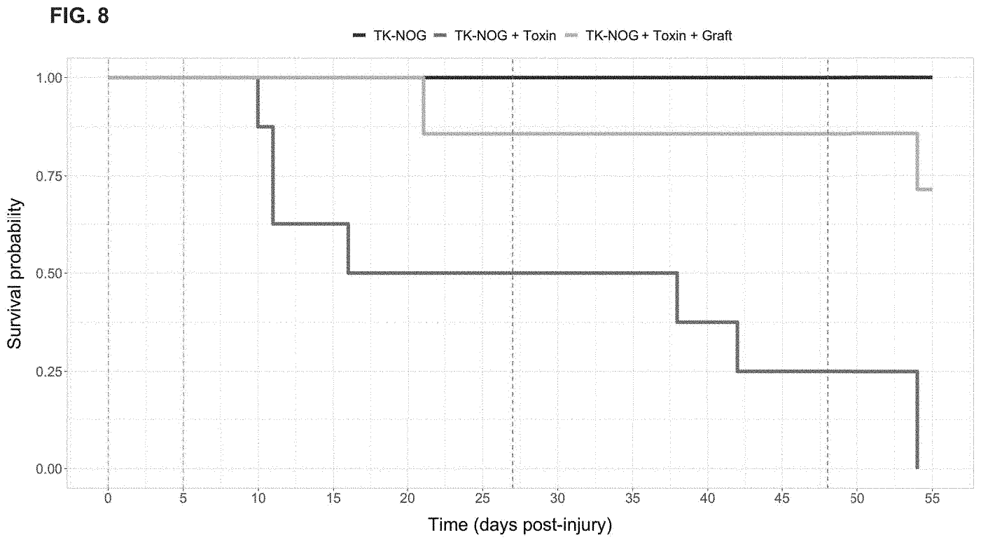

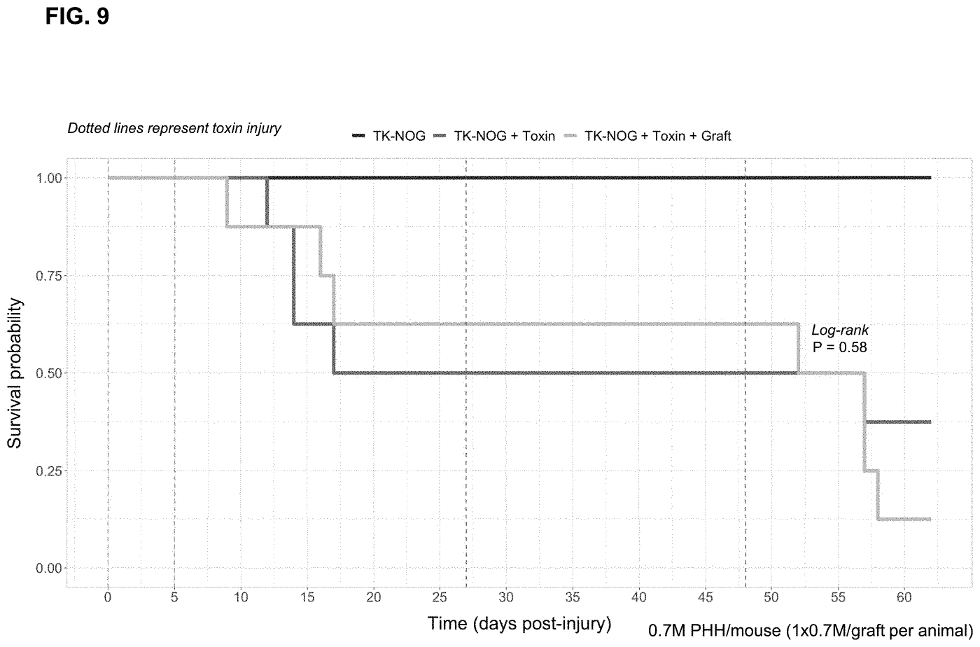

In evaluation of the levels of liver enzymes including alkaline phosphatase (ALP), alanine aminotransferase (ALT), and aspartate aminotransferase (AST), we observed that implantation of the high dose engineered tissue construct effectively normalized the levels of ALP, ALT, and AST, while the low dose engineered tissue construct elicited more modest effects (FIG. 7). In a probability of survival study, we observed that mice that were treated with GCV and implanted with a high-dose engineered tissue construct, exhibited an elevated probability to survive, whereas mice that received GCV alone, without the implantation of an engineered tissue construct, had a reduced probability to survive, with a sharp increase in mortality beginning on day 10 (FIG. 9). The low-dose engineered tissue construct was less effective in attenuating mortality (FIG. 8).

### Example 3. In Vivo Evaluation of Engineered Tissue Constructs in Immunocompetent Mice

To determine the effectiveness of engineered tissue constructs in an in vivo model of a urea cycle disorder, a study was performed with hypomorph transgenic mice having the sparse fur-abnormal skin and hair mutation (Otc^{spf-ash}) on the X chromosome. The Otc^{spf-ash} mutation results in the reduction of ornithine transcarbamylase (OTC), a critical enzyme in the urea cycle, activity in the liver. The reduction of hepatic OTC activity in this mouse model is usually from 5-10% compared to wild-type mice.

Otc^{spf-ash} transgenic mice were administered an immunosuppressant composition every two days. The immunosuppressant composition was administered every two days for six days prior to Day 1 of the study. One day before the start of the study, a blood draw was performed to obtain a baseline plasma ammonia concentration measurement. At Day 0, one experimental group was implanted with two engineered tissue constructs, each including 1.41×10⁶ PHH per graft, for a total of 2.82×10⁶ PHH per animal, whilst the other was not. At Week 1, the first of two NH₄Cl challenges was performed. 7.5 mmol/kg of NH₄Cl was administered to both groups via the i.p. route. The day prior to each NH₄Cl challenge, all animals had their bladders emptied, the urine was discarded, and an overnight fast was initiated. Blood samples (50 μl) were collected before each NH₄Cl challenge, 20 minutes after, and 40 minutes after each NH₄Cl challenge. The other NH₄Cl challenge was performed Week 4.

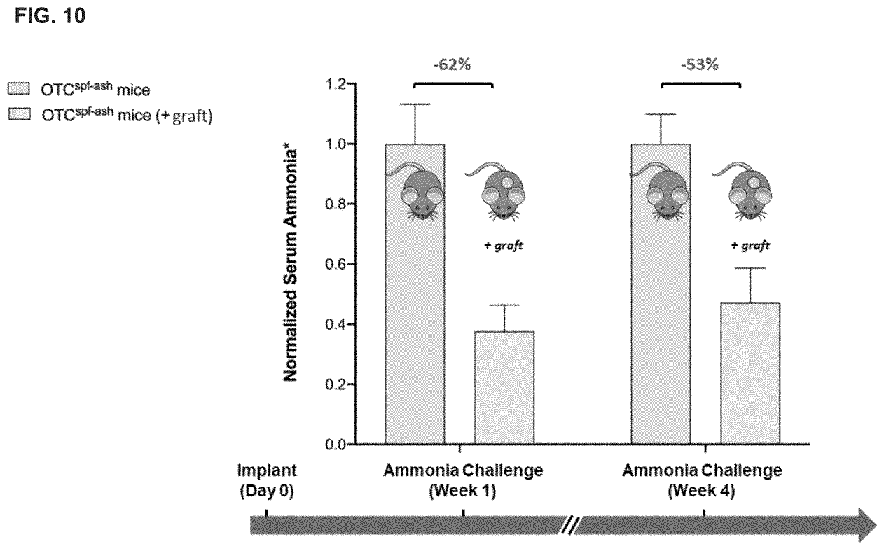

Otc^{spf-ash} that were implanted with the engineered tissue construct showed significant resilience to the ammonia challenges compared to Otc^{spf-ash} without an engineered tissue construct (FIG. 10).

### Example 4. Biomarker Development for Crigler-Najjar Syndrome Using Gunn Rat Model

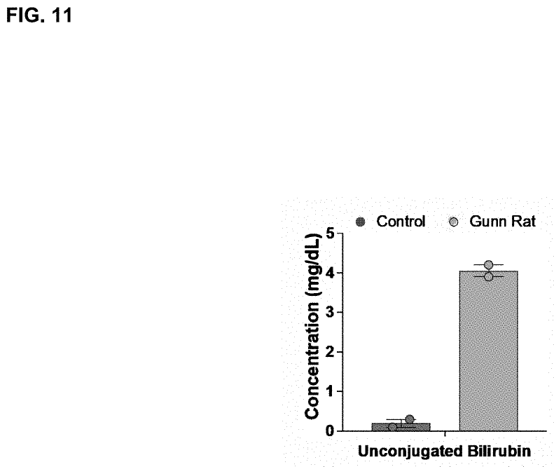

To determine whether unconjugated bilirubin could be used as a biomarker for Crigler-Najjar syndrome (e.g., Crigler-Najjar syndrome type I), the serum levels of unconjugated bilirubin were measured in homozygous Gunn rats and compared to the serum levels of unconjugated bilirubin in control Wistar rats. Gunn rats exhibited a minimum 4-fold increase of serum levels of unconjugated bilirubin compared to control Wistar rats, thereby establishing unconjugated bilirubin as a potential biomarker for Crigler-Najjar syndrome (FIG. 11).

### Example 5. Assessment of Albumin and Unconjugated Bilirubin in Gunn Rats

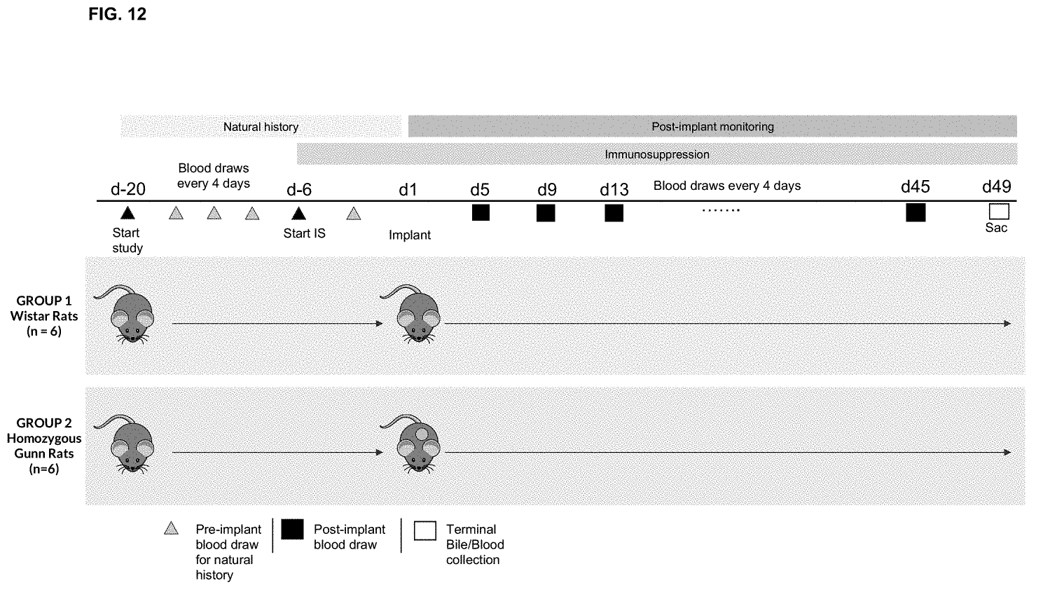

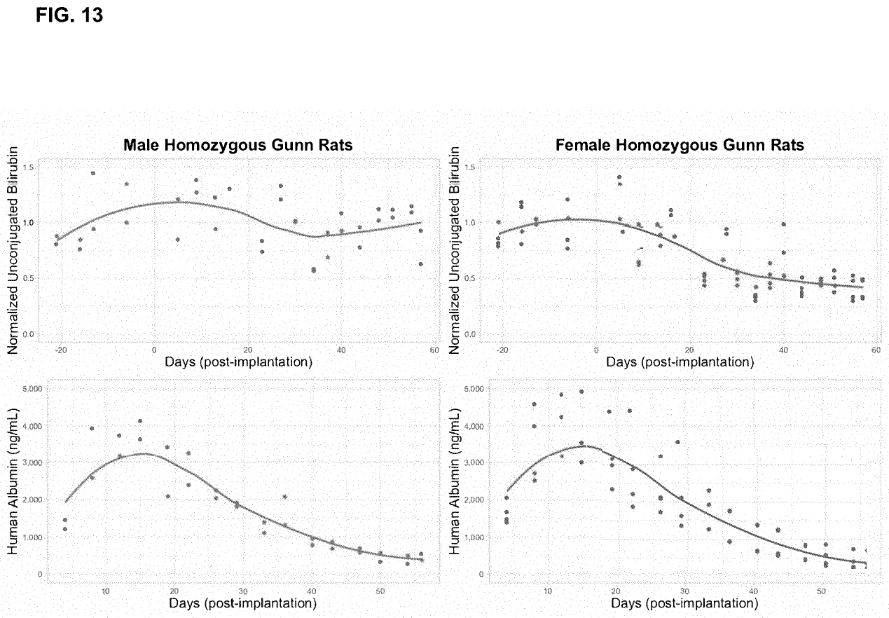

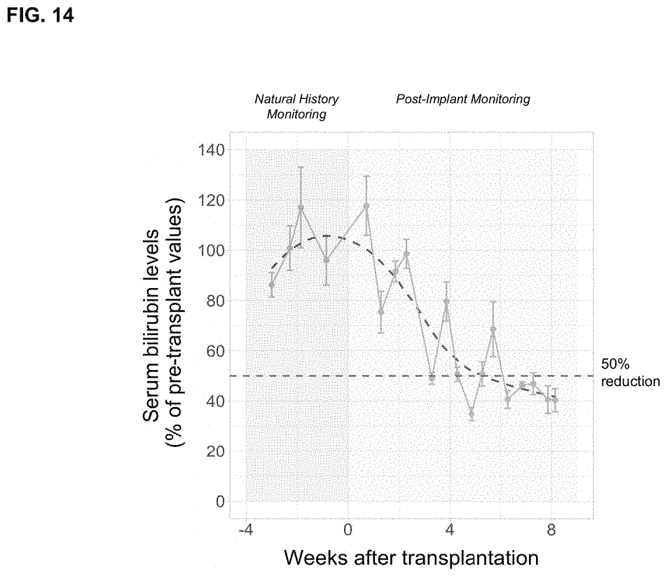

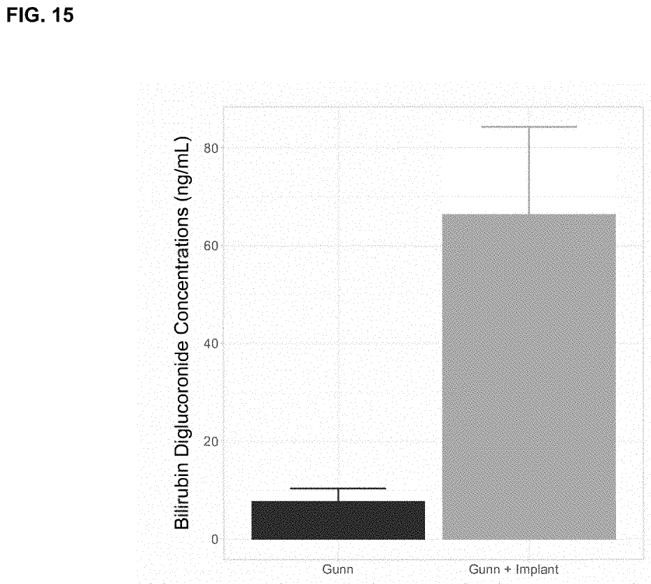

Homozygous Gunn rats were implanted with eight engineered tissue constructs including 1.41×10⁶ PHH and 2.82×10⁶ NHDF. Serum levels of unconjugated bilirubin and albumin were assessed by blood draw every fourth or fifth day, beginning from 20 days (p-20) pre-implantation to 49 days (d49) post-implantation (FIG. 12). Data were separated by sex and unconjugated bilirubin levels were normalized to control Wistar rats. Both unconjugated bilirubin and albumin serum levels decreased in homozygous Gunn rats post-implantation, regardless of sex (FIG. 13). Serum unconjugated bilirubin levels reduced by 50% within 5 weeks post-implantation (FIG. 14). Additionally, the presence of conjugated bilirubin products (bilirubin diglucuronide) in the bile was assessed in rats implanted with eight engineered tissue constructs, as compared to those without an engineered tissue constructs. Homozygous Gunn rats exhibited an increase in levels of conjugated bilirubin products in the bile relative to control Wistar rats (FIG. 15), confirming that the engineered tissue constructs of hepatocyte grafts partially rescued liver function by improving efficiency of bilirubin conjugation.

### Example 6. In Vivo Evaluation of Engineered Tissue Constructs in Immunocompetent Swine

To determine the effectiveness of engineered tissue constructs in an in vivo model, a study was performed with immunocompetent Yorkshire swine.

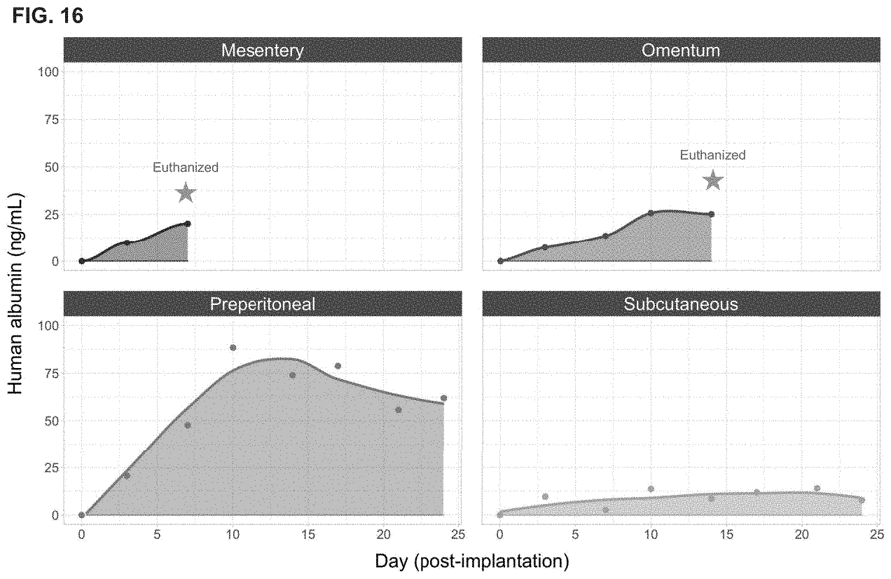

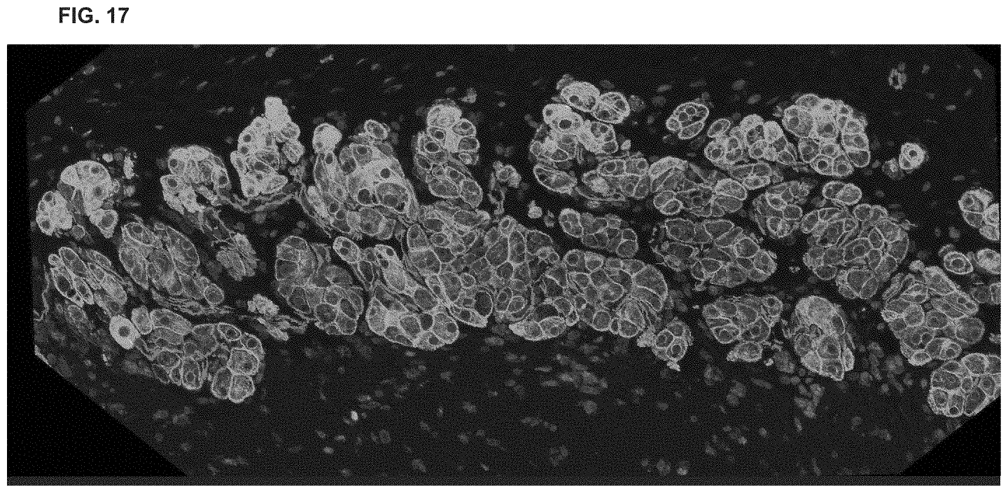

Swine implanted with engineered tissue constructs in the mesentery, omentum, preperitoneally, or subcutaneously, respectively, showed elevated levels of human albumin (ng/mL), with the preperitoneal implantation site supporting the strongest expression of human albumin (FIG. 16). Following triggered euthanasia, histological analyses of the explanted engineered tissue constructs revealed functioning expression of hepatocytes and host blood vessels in the graft region (FIG. 17). Taken together, these histological analyses revealed a large number of hepatocytes that survived (e.g., persisted).

### Example 7. Methods of Making an Engineered Tissue Construct

An engineered tissue construct suitable for implantation into a subject is made with a plurality of spheroids that include hepatocytes (e.g., PHH) and, optionally, stromal cells (e.g., fibroblasts e.g., NHDF or neonatal foreskin fibroblasts) in a biocompatible scaffold (e.g., fibrin). The engineered tissue construct may also include a reinforcing agent (e.g., fibrin, surgical mesh, alginate, collagen, poly(ethylene glycol), polyvinylidene acetate, polyvinylidene fluoride, poly(lactic-co-glycolic) acid, or poly (I-lactic acid)).

The population of hepatocytes may be from 3×10⁵ to 1.8×10¹¹ (e.g., from 4×10⁵ to 1.8×10¹¹ from 5×10⁵ to 1.8×10¹¹, from 6×10⁵ to 1.8×10¹¹, from 7×10⁵ to 1.8×10¹¹, from 8×10⁵ to 1.8×10¹¹, from 9×10⁵ to 1.8×10¹¹, from 1×10⁶ to 1.8×10¹¹, from 2×10⁶ to 1.8×10¹¹, from 3×10⁶ to 1.8×10¹¹, from 4×10⁶ to 1.8×10¹¹, from 5×10⁶ to 1.8×10¹¹, from 6×10⁶ to 1.8×10¹¹, from 7×10⁶ to 1.8×10¹¹, from 8×10⁶ to 1.8×10¹¹, from 9×10⁶ to 1.8×10¹¹, from 1×10⁷ to 1.8×10¹¹, from 2×10⁷ to 1.8×10¹¹, from 1.8×10⁷ to 1.8×10¹¹, from 4×10⁷ to 1.8×10¹¹, from 5×10⁷ to 1.8×10¹¹, from 6×10⁷ to 1.8×10¹¹, from 7×10⁷ to 1.8×10¹¹, from 8×10⁷ to 1.8×10¹¹, from 9×10⁷ to 1.8×10¹¹, from 1×10⁸ to 1.8×10¹¹, from 2×10⁸ to 1.8×10¹¹, from 3×10⁸ to 1.8×10¹¹, from 4×10⁸ to 1.8×10¹¹, from 5×10⁸ to 1.8×10¹¹, from 6×10⁸ to 1.8×10¹¹, from 7×10⁸ to 1.8×10¹¹, from 8×10⁸ to 1.8×10¹¹, from 9×10⁸ to 1.8×10¹¹, from 1×10⁹ to 1.8×10¹¹, from 2×10⁹ to 1.8×10¹¹, from 3×10⁹ to 1.8×10¹¹, from 4×10⁹ to 1.8×10¹¹, from 5×10⁹ to 1.8×10¹¹, from 6×10⁹ to 1.8×10¹¹, from 7×10⁹ to 1.8×10¹¹, from 8×10⁹ to 1.8×10¹¹, from 9×10⁹ to 1.8×10¹¹, from 1×10¹⁰ to 1.8×10¹¹, from 2×10¹⁰ to 1.8×10¹¹, from 3×10¹⁰ to 1.8×10¹¹, from 4×10¹⁰ to 1.8×10¹¹, from 5×10¹⁰ to 1.8×10¹¹, from 6×10¹⁰ to 1.8×10¹¹, from 7×10¹⁰ to 1.8×10¹¹, from 8×10¹⁰ to 1.8×10¹¹, from 9×10¹⁰ to 1.8×10¹¹, or from 1×10¹¹ to 1.8×10¹¹) cell. The optional population of stromal cells may be between 0 to 1.8×10¹² (e.g., from 1 to 1.8×10¹², from 10 to 1.8×10¹², from 100 to 1.8×10¹², from 1×10³ to 1.8×10¹², from 2×10³ to 1.8×10¹², from 3×10³ to 1.8×10¹², from 4×10³ to 1.8×10¹², from 5×10³ to 1.8×10¹², from 6×10³ to 1.8×10¹², from 7×10³ to 1.8×10¹², from 8×10³ to 1.8×10¹², from 9×10³ to 1.8×10¹², from 1×10⁴ to 1.8×10¹², from 2×10⁴ to 1.8×10¹², from 3×10⁴ to 1.8×10¹², from 4×10⁴ to 1.8×10¹², from 5×10⁴ to 1.8×10¹², from 6×10⁴ to 1.8×10¹², from 7×10⁴ to 1.8×10¹², from 8×10⁴ to 1.8×10¹², from 9×10⁴ to 1.8×10¹², from 1×10⁵ to 1.8×10¹², from 2×10⁵ to 1.8×10¹², from 3×10⁵ to 1.8×10¹², from 4×10⁵ to 1.8×10¹², from 5×10⁵ to 1.8×10¹², from 6×10⁵ to 1.8×10¹², from 7×10⁵ to 1.8×10¹², from 8×10⁵ to 1.8×10¹², from 9×10⁵ to 1.8×10¹², from 1×10⁶ to 1.8×10¹², from 2×10⁶ to 1.8×10¹², 3×10⁶ to 1.8×10¹², 4×10⁶ to 1.8×10¹², 5×10⁶ to 1.8×10¹², 6×10⁶ to 1.8×10¹², 7×10⁶ to 1.8×10¹², 8×10⁶ to 1.8×10¹², 9×10⁶ to 1.8×10¹², from 1×10⁷ to 1.8×10¹², from 2×10⁷ to 1.8×10¹², from 18×10⁷ to 1.8×10¹², from 4×10⁷ to 1.8×10¹², from 5×10⁷ to 1.8×10¹², from 6×10⁷ to 1.8×10¹², from 7×10⁷ to 1.8×10¹², from 8×10⁷ to 1.8×10¹², from 9×10⁷ to 1.8×10¹², from 1×10⁸ to 1.8×10¹², from 2×10⁸ to 1.8×10¹², from 3×10⁸ to 1.8×10¹², from 4×10⁸ to 1.8×10¹², from 5×10⁸ to 1.8×10¹², from 6×10⁸ to 1.8×10¹², from 7×10⁸ to 1.8×10¹², from 8×10⁸ to 1.8×10¹², from 9×10⁸ to 1.8×10¹², from 1×10⁹ to 1.8×10¹², from 2×10⁹ to 1.8×10¹², from 3×10⁹ to 1.8×10¹², from 4×10⁹ to 1.8×10¹², from 5×10⁹ to 1.8×10¹², from 6×10⁹ to 1.8×10¹², from 7×10⁹ to 1.8×10¹², from 8×10⁹ to 1.8×10¹², from 9×10⁹ to 1.8×10¹², from 1×10¹⁰ to 1.8×10¹², from 2×10¹⁰ to 1.8×10¹², from 3×10¹⁰ to 1.8×10¹², from 4×10¹⁰ to 1.8×10¹², from 5×10¹⁰ to 1.8×10¹², from 6×10¹⁰ to 1.8×10¹², from 7×10¹⁰ to 1.8×10¹², from 8×10¹⁰ to 1.8×10¹², from 9×10¹⁰ to 1.8×10¹², or from 1×10¹¹ to 1.8×10¹²) cells.

The population of hepatocytes and the population of stromal cells, together, account for at least 70% (e.g., at least 71%, 72%, 73%, 74%, 75%, 80%, 85%, 90%, 95%, or 100%) of the total cells in the engineered tissue construct. In some embodiments, the engineered tissue construct further includes other cell types (e.g., endothelial cells e.g., up to 30% of the cells in the engineered tissue construct are endothelial cells). The density of the hepatocytes in the engineered tissue construct may be from, for example, 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). The ratio of hepatocytes to stromal cells may be between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

The biocompatible scaffold may have an x-axis, a y-axis, and a z-axis (e.g., the z-axis may be 500 μm to 5 mm (e.g., 600 μm to 4 mm, 700 μm to 3 mm, 800 μm to 2 mm, or 900 μm to 1 mm)). The hepatocytes and stromal cell aggregates are allowed to distribute non-homogenously along the z-axis of the biocompatible scaffold (e.g., by gravity), simultaneously during the step of encapsulation with, for example, a fibrinogen solution that is polymerized with thrombin. The aggregates may be distributed homogeneously in the x-axis and/or y-axis. The biocompatible scaffold polymerizes in less than 3 hours (e.g., less than 2 hours, 1 hour, 59 minutes, 58 minutes, 57 minutes, 56 minutes, 55 minutes, 50 minutes, 45 minutes, 40 minutes, 30 minutes, 20 minutes, 10 minutes, or 5 minutes. Furthermore, the engineered tissue construct may be from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

The layer may be from 100 μm to 1 mm (e.g., 200 μm to 900 μm, 300 μm to 800 μm, 400 μm to 700 μm, or 500 μm to 600 μm) thick and/or the density of hepatocytes in the layer is from 0.06 M/cm² to 150 M/cm² (e.g., 0.07 M/cm² to 149 M/cm², 0.08 M/cm² to 148 M/cm², 0.09 M/cm² to 147 M/cm², 0.1 M/cm² to 146 M/cm², 0.2 M/cm² to 145 M/cm², 0.3 M/cm² to 140 M/cm², 0.4 M/cm² to 130 M/cm², 0.5 M/cm² to 120 M/cm², 1 M/cm² to 110 M/cm², 2 M/cm² to 100 M/cm², 3 M/cm² to 50 M/cm², 4 M/cm² to 40 M/cm², 5 M/cm² to 30 M/cm², or 10 M/cm² to 20 M/cm²). Furthermore, the ratio of height of the biocompatible scaffold to height of the layer may be from 20:1 to 1:1 e.g., 19:1 to 1:1, 18:1 to 1:1, 17:1 to 1:1, 16:1 to 1:1, 15:1 to 1:1, 14:1 to 1:1, 13:1 to 1:1, 12:1 to 1:1, 11:1 to 1:1, 10:1 to 1:1, 9:1 to 1:1, 8:1 to 1:1, 7:1 to 1:1, 6:1 to 1:1, 5:1 to 1:1, 4:1 to 1:1, 3:1 to 1:1, or 2:1 to 1:1.

Afterwards, one or more engineered tissue constructs may be assembled with biocompatible glue (e.g., fibrin glue) such that each of the aggregate layers is outward facing, thereby forming a two-sided engineered tissue construct. Two-sided engineered tissue constructs may be produced in other ways. For example, they may be assembled surgically in situ.

### Example 8. Anatomic Site Measurement

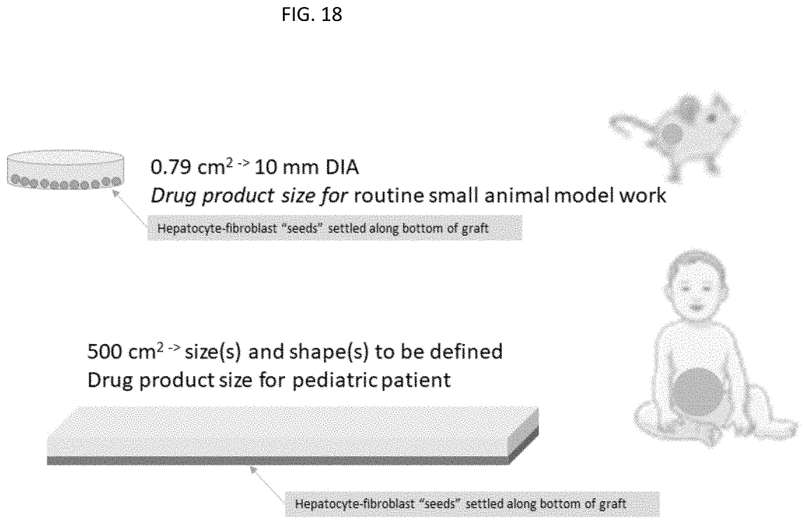

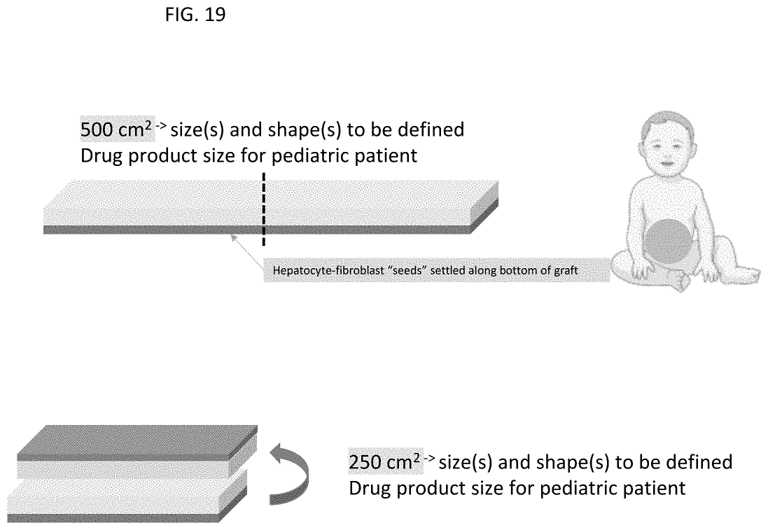

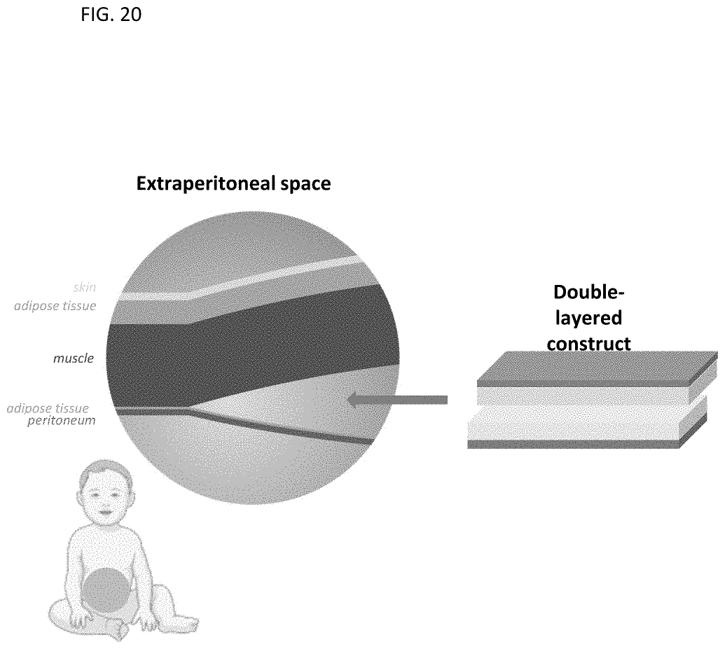

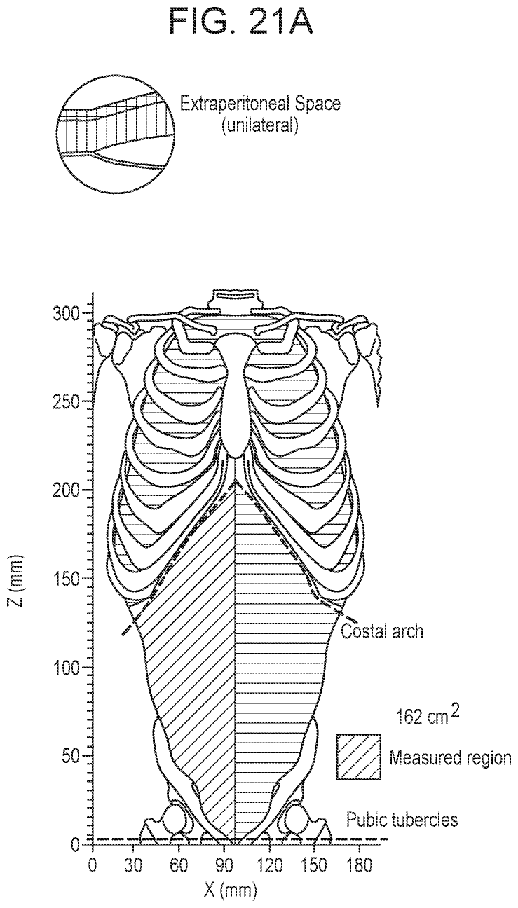

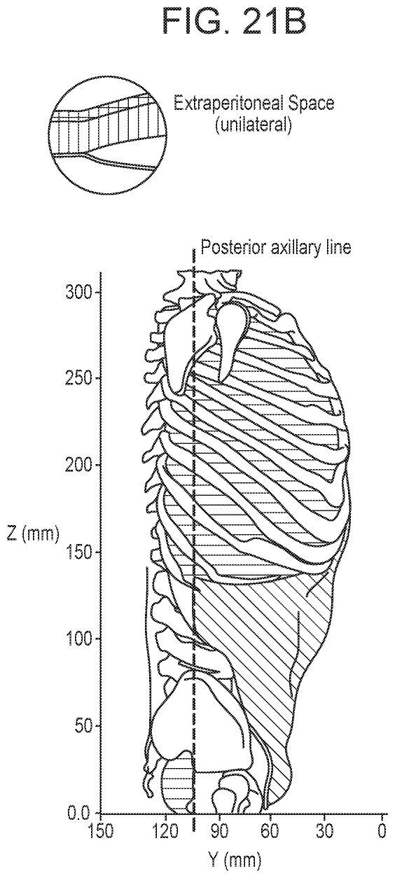

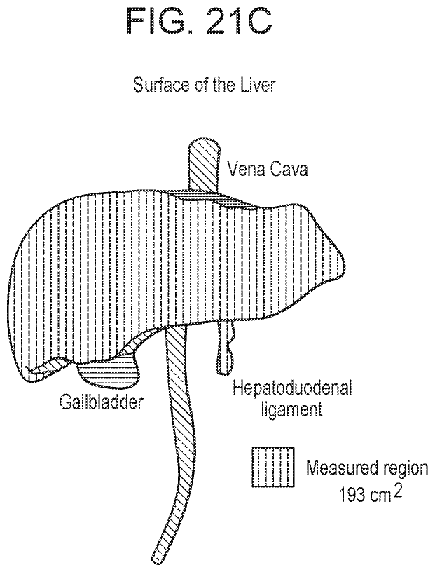

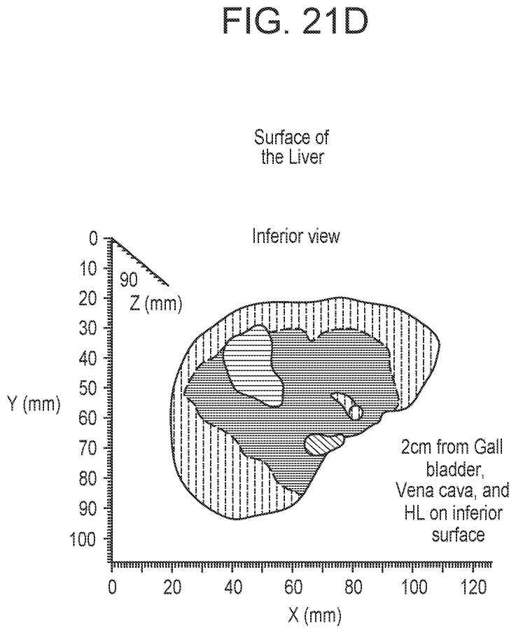

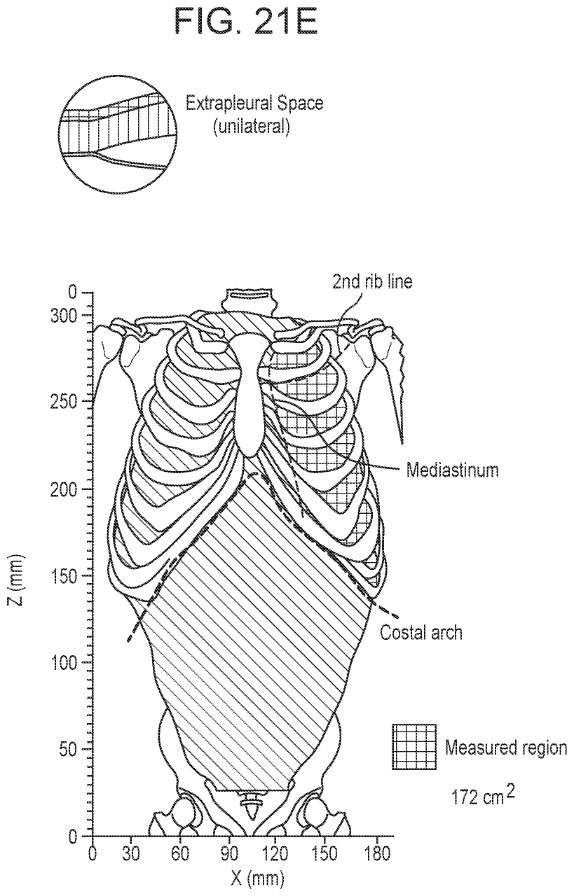

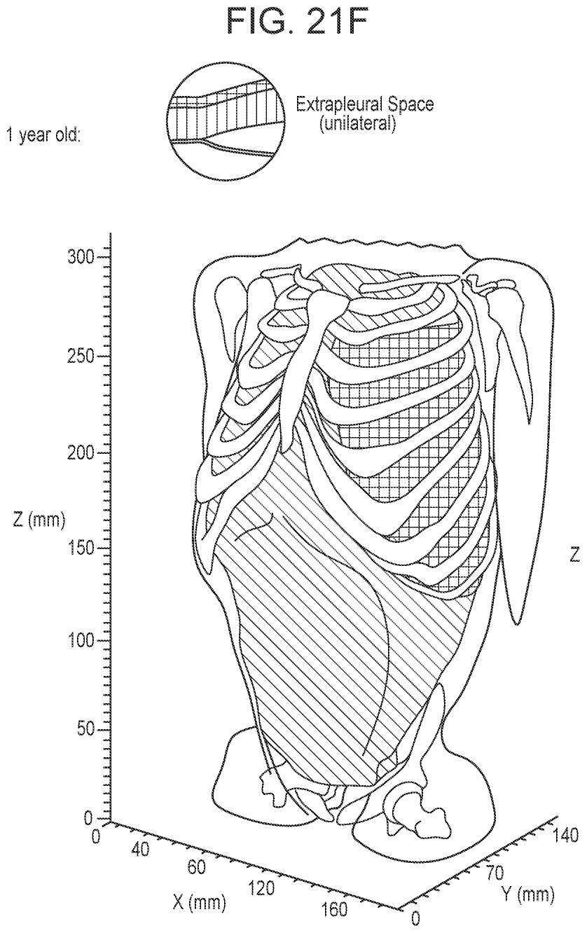

An engineered tissue construct may include seeds (hepatocyte-fibroblast spheroids) embedded in a fibrin hydrogel, with 12 M PHH/mL of graft with a double-sided assembly. An estimated dose for a one-year-old child is 1.2 B PHH. Therefore, a single-sided product may require 500 cm² of implantable surface area, while a two-sided product may require 250 cm² of implantable surface area. If an anatomic site has two surfaces for graft integration, the performance of a double-layered graft may be assessed (FIGS. 18-20). Three-dimensional models were used to understand the available implantable space for the extraperitoneal space, extrapleural space, and a surface of the liver (which encompasses but extends beyond the bare area of the liver (i.e., the non-peritonealized surface of the liver where it attaches to the diaphragm). Space assessments were performed for the following patient models: 36th weight percentile of 2-month-old newborn and 53rd weight percentile of a 1-year old child (FIGS. 21A-21F). FIGS. 21A-21F depicts unilateral space available for implantation. However, engineered tissue constructs may also be implanted at these spaces bilaterally. Based on these models, the total available space was calculated as follows as shown in Table 1.

TABLE 1
Anatomic site measurement

| | Best Graft | Total space available |
|---|---|---|
| | Geometry | (1-year old, |
| Anatomic Site | Option | 53^{rd} weight percentile) |
| Extraperitoneal | Double-layered | 324 cm² (bilateral) |
| space | configuration | |
| | compatible | |
| Liver | Single-layered | 193 cm² |
| | configuration | |
| Extrapleural | Single-layered | 344 cm² (bilateral) |
| | configuration | |

### Example 9. Seed Orientation Study

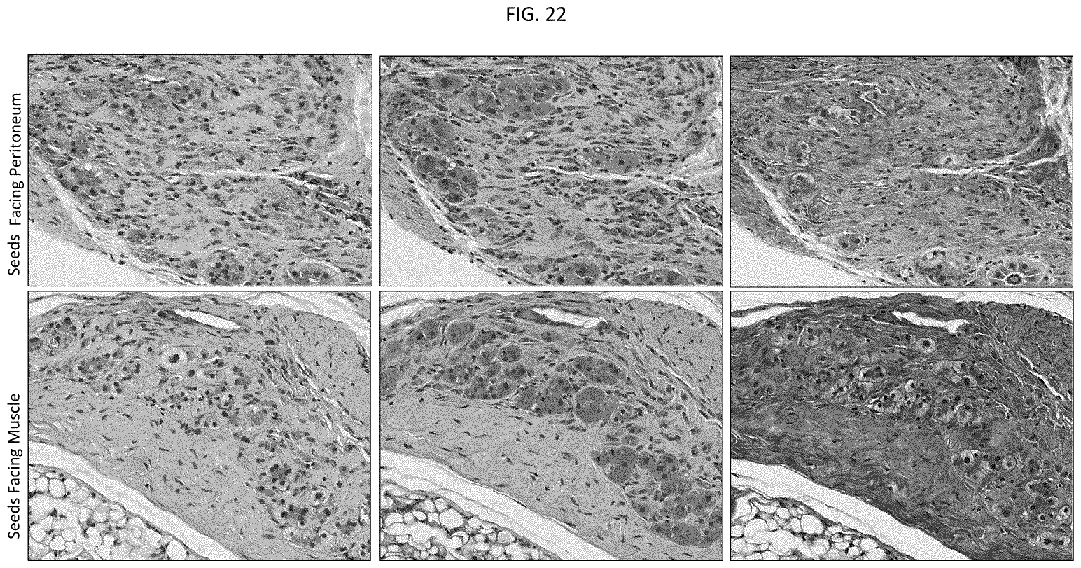

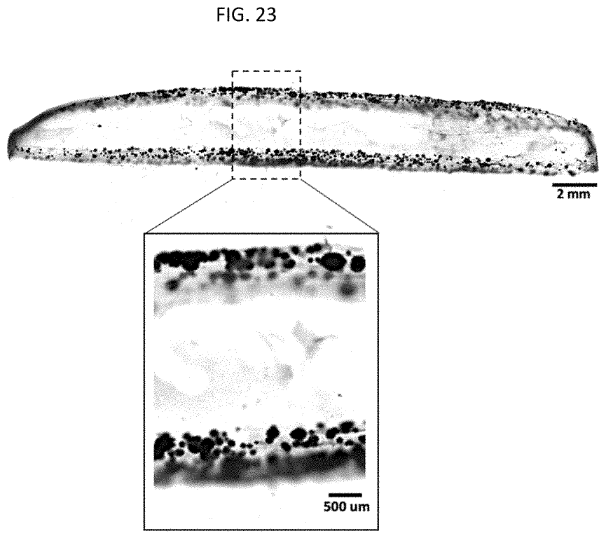

The effects of orientation of the seeds of cells relative to the implantation site were assessed for extraperitoneal implantation. The site of implantation was dissected down through skin, fat, connective tissue and muscle to the peritoneum. Blunt dissection of connective tissue between the peritoneum and muscle was performed to form an implant pocket. Separate grafts placed side by side between the muscle fascia and peritoneum in the extraperitoneal space. The grafts were placed into the extraperitoneal space either with the cell side towards the muscle or cells toward the peritoneum. The muscle fascia was then sutured to the peritoneum in between the grafts to prevent any graft overlap (the grafts themselves were not directly sutured). 25 mm grafts were explanted from the peritoneum. The cells exhibited an adhesive characteristic and adhered to the tissue against which they were implanted, indicating integration. Other grafts were explanted from the muscle. Graft adhesion in extraperitoneal space on explant was based on implant cell side adhesive characteristics. The graft size was slightly reduced (25 mm to 17 mm) by the time of explant but maintained its shape integrity (circular). No obvious inferiority was detected between the seed orientation options tested regarding hepatocyte cell health (FIG. 22).

### Example 10. Double-Layer Engineered Tissue Construct

In this Example, double-layer engineered tissue constructs are described. As is discussed, e.g., in Example 8, engineered tissue constructs with two layers of cells may be utilized. Further, as is shown in Example 9, for implantation into the extraperitoneal space, engineered tissue constructs may be placed either with the cell side towards the muscle or cells toward the peritoneum without any obvious effects on hepatocyte cell health.

FIG. 23 shows a photomicrograph of a sagittal cross-section taken from a double-layer engineered tissue construct composed of two biocompatible fibrin hydrogel layers, each of which are seeded with hepatocytes and stromal cell spheroids distributed along the entire outer surface of the tissue construct.

### Ordered Embodiments

The following sections describe various embodiments of the invention.

### Embodiment A

1. A method for implanting an engineered tissue construct comprising a population of mammalian cells in a biocompatible scaffold, the method comprising implanting the engineered tissue construct in a human subject in an extraperitoneal space, in an extrapleural space, on a surface of the liver, in a muscle site, in a pleural space, in an omentum site, in a subcutaneous site, on a surface of the pancreas, on a surface of the spleen, on a surface of the kidney, in a bone marrow site, in a bursa site, in a peritoneal site, and/or in a lesser sac site.

2. The method of embodiment 1, wherein upon implantation the population of cells are engrafted and vascularized in the subject.

3. The method of embodiment 1 or 2, wherein the population of cells comprises primary cells, induced pluripotent cell (iPSC)-derived cells, embryonic stem cell-derived cells, engineered cells, cell aggregates, or a tissue or portion thereof.

4. The method of embodiment 3, wherein the primary cells comprise primary cells expanded in vitro.

5. The method of embodiment 3, wherein the engineered cells are engineered to express or secrete a protein.

6. The method of embodiment 5, wherein the protein comprises an antibody, a cytokine, an enzyme, a coagulation factor, or a hormone.

7. The method of embodiment 5 or 6, wherein the protein is an endogenous human protein or an engineered protein.

8. The method of any one of embodiments 1-7, wherein the engineered tissue construct is implanted in the extraperitoneal space, the extrapleural space, or the surface of the liver.

9. The method of embodiment 8, wherein the engineered tissue construct is implanted in the extraperitoneal space.

10. The method of embodiment 9, wherein the extraperitoneal space is a pre-peritoneal space, a retroperitoneal space, or a subperitoneal space.

11. The method of embodiment 8, wherein the engineered tissue construct is implanted on the surface of the liver.

12. The method of any one of embodiments 1-11, wherein the muscle site is on a surface of a muscle, within a muscle sheath, or beneath a muscle.

13. The method of embodiment 12, wherein the muscle site is on a surface of a muscle.

14. The method of embodiment 12, wherein the muscle site is within a muscle sheath.

15. The method of embodiment 12, wherein the muscle site is beneath a muscle.

16. The method of embodiment 11, wherein the engineered tissue construct is layered on the dome of the liver and/or covered with omentum.

17. The method of any one of embodiments 1-7, wherein the omentum site comprises an omentum pedicle flap, an omentum free flap, an omental bursa, or the omentum in situ.

18. The method of any one of embodiments 1-7, wherein the engineered tissue construct is implanted subcutaneously with an omental flap, subcutaneously with an adjuvant, or subcutaneously with an arteriovenous fistula.

19. The method of any one of embodiments 1-7, wherein the muscle is a rectus abdominis, an abdominal oblique, a transversus abdominus, a quadriceps femoris, a gluteus maximus, a semimembranosus, a semitendinosus, a biceps femoris, a deltoid, a biceps, or a latissimus dorsi.

20. The method of any one of embodiments 1-19, wherein the population of cells comprises endocrine, exocrine, paracrine, heterocrine, autocrine, or juxtacrine cells.

21. The method of any one of embodiments 1-20, wherein the population of cells comprises leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, enteroendocrine cells (e.g., L cells and/or chromaffin cells), ovarian cells, parathyroid cells, thyroid cells, and/or neuronal cells.

22. The method of embodiment 21, wherein the pituitary cells comprise thyrotropic pituitary cells, lactotropic pituitary cells, corticotropic pituitary cells, somatotropic pituitary cells, and/or gonadotropic pituitary cells.

23. The method of embodiment 21, wherein the neuronal cells comprise dopaminergic cells. 24. The method of any one of embodiments 1-21, wherein the population of cells comprises a population of hepatocytes and a population of stromal cells.

25. The method of embodiment 24, wherein the hepatocytes comprise primary human hepatocytes.

26. The method of embodiment 24 or 25, wherein the stromal cells comprise fibroblasts. 27. The method of embodiment 26, wherein the fibroblasts are normal human dermal fibroblasts or neonatal foreskin fibroblasts.

28. The method of any one of embodiments 24-27, wherein the engineered tissue construct further comprises a population of endothelial cells.

29. The method of embodiment 28, wherein the population of endothelial cells is arranged as one or more cords.

30. The method of any one of embodiments 1-29, wherein the population of cells comprises human cells.

31. The method of any one of embodiments 1-30, wherein the biocompatible scaffold comprises fibrin.

32. The method of any one of embodiments 1-31, wherein the engineered tissue construct further comprises a reinforcing agent.

33. The method of embodiment 32, wherein the reinforcing agent comprises fibrin, surgical mesh, alginate, collagen, poly(ethylene glycol), polyvinylidene acetate, polyvinylidene fluoride, poly(lactic-co-glycolic) acid, or poly (I-lactic acid).

34. The method of any one of embodiments 1-33, wherein the engineered tissue construct has a surface area of 10 cm² to 2,000 cm².

35. The method of any one of embodiments 1-34, wherein the cells are located on a first face of the engineered tissue construct.

36. The method of embodiment 35, wherein the first face of the engineered tissue construct contacts a site of implantation

37. The method of any one of embodiments 1-36, wherein the cells are located on a first face and a second face of the engineered tissue construct.

38. The method of any one of embodiments 1-37, wherein the engineered tissue construct is triangular, rectangular, or circular.

39. The method of any one of embodiments 1-38, wherein the method comprises implanting a plurality of engineered tissue constructs.

40. The method of embodiment 39, wherein each of the plurality of engineered tissue constructs is implanted in a different site.

41. The method of any one of embodiments 24-40, wherein the method treats acute liver failure, a urea cycle disorder, Crigler-Najjar syndrome, diabetes, an endocrine disorder, a hormonal deficiency, a protein deficiency, impaired biotransformation, or a disease of impaired protein synthesis.

42. The method of any one of embodiments 1-41, wherein the subject has an age of between 1 day and 120 years.

43. The method of embodiment 42, wherein the subject has an age of between 1 day and 1 year. 44. The method of any one of embodiments 1-43, wherein the engineered tissue construct is implanted using an open surgical procedure or a minimally invasive surgery.

45. The method of any one of embodiments 1-44, wherein the engineered tissue construct is affixed by one or more sutures or one or more staples.

46. The method of embodiment 45, wherein the engineered tissue construct is affixed by suturing adjoining tissue to restrain migration of the engineered tissue construct.

47. The method of claim 46, wherein the engineered tissue construct is implanted at an extraperitoneal site, and the engineered tissue construct is affixed by suturing the muscle fascia to the peritoneum at one or more positions surrounding the engineered tissue construct.

48. The method of any one of embodiments 45-47, wherein the engineered tissue construct is not directly sutured.

### Embodiment B

1. A method for implanting an engineered tissue construct comprising a population of human cells in a biocompatible scaffold, the method comprising implanting the engineered tissue construct in a human subject in an extraperitoneal space, in an extrapleural space, or on a liver surface, wherein the population of human cells comprises a population of hepatocytes.

2. The method of embodiment 1, wherein the population of human cells comprises a population of hepatocytes and a population of stromal cells.

3. The method of embodiment 1, wherein upon implantation the population of cells are engrafted and vascularized in the subject.

4. The method of embodiment 1, wherein the population of human cells comprises primary cells, engineered cells, cell aggregates, induced pluripotent stem cell derived cells, embryonic stem cell derived cells, transdifferentiated cells, or a tissue or portion thereof.

5. The method of embodiment 4, wherein the engineered cells are engineered to express or secrete a protein.

6. The method of embodiment 5, wherein the protein comprises an antibody, a cytokine, an enzyme, a coagulation factor, or a hormone.

7. The method of embodiment 5, wherein the protein is an endogenous human protein or an engineered protein.

8. The method of embodiment 1, wherein the engineered tissue construct is implanted in the extraperitoneal space.

9. The method of embodiment 8, wherein the extraperitoneal space is a pre-peritoneal space, a retroperitoneal space, or a subperitoneal space.

10. The method of embodiment 1, wherein the engineered tissue construct is implanted on the surface of the liver.

11. The method of embodiment 10, wherein the engineered tissue construct is layered on the dome of the liver and/or covered with omentum.

12. The method of embodiment 1, wherein the hepatocytes comprise primary human hepatocytes.

13. The method of embodiment 2, wherein the stromal cells comprise fibroblasts.

14. The method of embodiment 13, wherein the fibroblasts are normal human dermal fibroblasts or neonatal foreskin fibroblasts.

15. The method of embodiment 1, wherein the engineered tissue construct further comprises a population of endothelial cells.

16. The method of embodiment 15, wherein the population of endothelial cells is arranged as one or more cords.

17. The method of embodiment 1, wherein the biocompatible scaffold comprises fibrin.

18. The method of embodiment 1, wherein the engineered tissue construct further comprises a reinforcing agent.

19. The method of embodiment 18, wherein the reinforcing agent comprises fibrin, surgical mesh, alginate, collagen, poly(ethylene glycol), polyvinylidene acetate, polyvinylidene fluoride, poly(lactic-co-glycolic) acid, or poly (I-lactic acid).

20. The method of embodiment 1, wherein the engineered tissue construct has a surface area of 10 cm² to 2,000 cm².

21. The method of embodiment 1, wherein the engineered tissue construct comprises between 1×10⁶ hepatocytes/mL and 100×10⁶ hepatocytes/mL.

22. The method of embodiment 1, wherein the engineered tissue construct has a volume of between 20 mL and 1.2 L.

23. The method of embodiment 1, wherein the cells are located on a first face of the engineered tissue construct.

24. The method of embodiment 23, wherein the first face of the engineered tissue construct contacts a site of implantation

25. The method of embodiment 1, wherein the cells are located on a first face and a second face of the engineered tissue construct.

26. The method of embodiment 1, wherein the method comprises implanting a plurality of engineered tissue constructs.

27. The method of embodiment 26, wherein each of the plurality of engineered tissue constructs is implanted in a different site.

28. The method of embodiment 1, wherein the method treats a liver disease.

29. The method of embodiment 28, wherein the liver disease is acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4 (Andersen), hepatic encephalopathy, Hunter syndrome (MPS II), infantile refsum disease, lysosomal acid lipase deficiency (LAL-D) (cholesteryl ester storage disease), maple syrup urine disease, Maroteaux-Lamy (MPS VI), methylmalonic acidemia, ornithine transcarbamylase (OTC) deficiency, propionic acidemia, or a urea cycle disorder.

30. The method of embodiment 2, wherein the method comprises implanting the engineered tissue construct in a human subject in an extraperitoneal space, the population of cells comprises a population of hepatocytes and a population of stromal cells, and the biocompatible scaffold comprises fibrin.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method for implanting an engineered tissue construct comprising a population of human cells comprising a population of hepatocytes and a population of stromal cells in a biocompatible scaffold that comprises human fibrin, the method comprising forming an implant pocket between a peritoneum and a muscle tissue of a human subject, and implanting the engineered tissue construct in the implant pocket.

2-3. (canceled)

4. The method of claim 1, wherein upon implantation the population of cells are engrafted and vascularized in the subject.

5. The method of claim 1, wherein the population of human cells comprises primary cells, engineered cells, cell aggregates, induced pluripotent stem cell derived cells, embryonic stem cell derived cells, transdifferentiated cells, or a tissue or portion thereof.

6. The method of claim 5, wherein the engineered cells are engineered to express or secrete a protein.

7. The method of claim 6, wherein the protein is an antibody, a cytokine, an enzyme, a coagulation factor, or a hormone.

8. The method of claim 6, wherein the protein is an endogenous human protein or an engineered protein.

9. The method of claim 1, wherein the engineered tissue construct is implanted in an extraperitoneal space.

10. The method of claim 9, wherein the extraperitoneal space is a pre-peritoneal space, a retroperitoneal space, or a subperitoneal space.

11-12. (canceled)

13. The method of claim 1, wherein the hepatocytes comprise primary human hepatocytes.

14. The method of claim 1, wherein the stromal cells comprise fibroblasts.

15. The method of claim 14, wherein the fibroblasts are normal human dermal fibroblasts or neonatal foreskin fibroblasts.

16. The method of claim 1, wherein the engineered tissue construct further comprises a population of endothelial cells.

17. The method of claim 16, wherein the population of endothelial cells is arranged as one or more cords.

18. (canceled)

19. The method of claim 1, wherein the engineered tissue construct further comprises a reinforcing agent.

20. The method of claim 19, wherein the reinforcing agent comprises fibrin, surgical mesh, alginate, collagen, poly(ethylene glycol), polyvinylidene acetate, polyvinylidene fluoride, poly(lactic-co-glycolic) acid, or poly (I-lactic acid).

21. The method of claim 1, wherein the engineered tissue construct has a surface area of 10 cm² to 2,000 cm².

22. The method of claim 1, wherein the engineered tissue construct comprises between 1×10⁶ hepatocytes/mL and 1×10⁸ hepatocytes/mL.

23. The method of claim 1, wherein the engineered tissue construct has a volume of between 20 mL and 1.2 L.

24. The method of claim 1, wherein the cells are located on a first face of the engineered tissue construct.

25. The method of claim 24, wherein the first face of the engineered tissue construct contacts a site of implantation.

26. The method of claim 1, wherein the cells are located on a first face and a second face of the engineered tissue construct.

27. The method of claim 1, wherein the method comprises implanting a plurality of engineered tissue constructs.

28. The method of claim 27, wherein each of the plurality of engineered tissue constructs is implanted in a different site.

29. The method of claim 1, wherein the method treats a liver disease.

30. The method of claim 29, wherein the liver disease is acute liver failure, acute-on-chronic liver failure, congenital bile acid synthesis defect, Crigler-Najjar syndrome, end stage liver disease, familial hypercholesterolemia, familial hypobetalipoproteinemia, glycogen storage disorder type 1a, glycogen storage disorder type 4, hepatic encephalopathy, Hunter syndrome, infantile refsum disease, lysosomal acid lipase deficiency, maple syrup urine disease, Maroteaux-Lamy, methylmalonic acidemia, ornithine transcarbamylase deficiency, propionic acidemia, or a urea cycle disorder.
